(19) United States
(12) Patent Application Publication
(10) Pub. No.: US 2026/0317296 A1
(43) Pub. Date: Oct. 15, 2026

(54) HETEROCYCLIC COMPOUNDS FOR IMAGING AGGREGATED TAU IN TAUOPATHIES

(71) Applicants: University of Pittsburgh - Of the Commonwealth System of Higher Education, Pittsburgh, PA (US); The Trustees of the University of Pennsylvania, Philadelphia, PA (US)

(72) Inventors: Chester A. Mathis,, Jr., Pittsburgh, PA (US); Jeffrey S. Stehouwer, Pittsburgh, PA (US); Robert H. Mach, Wallingford, PA (US); Thomas J. A. Graham, Philadelphia, PA (US)

(73) Assignees: University of Pittsburgh - Of the Commonwealth System of Higher Education, Pittsburgh, PA (US); The Trustees of the University of Pennsylvania, Philadelphia, PA (US)

(21) Appl. No.: 19/482,918

(22) PCT Filed: May 10, 2024
(86) PCT No.: PCT/US2024/028756

§ 371 Date: Nov. 10, 2025

Related U.S. Application Data
(60) Provisional application No. 63/465,817, filed on May 11, 2023.

(51) Int. Cl. C07D 401/04; A61K 51/04; A61P 25/28; C07B 59/00; C07D 401/14; C07D 403/04; C07D 403/14

(52) U.S. Cl. CPC C07D 401/04; A61K 51/0455; A61K 51/0459; A61K 51/0463; A61P 25/28; C07B 59/002; C07D 401/14; C07D 403/04; C07D 403/14

(57) ABSTRACT

Provided are heterocyclic compounds of Formula I or Formula II or their pharmaceutically pharmaceutically acceptable salts, solvates, hydrates, tautomers or stereoisomers for imaging tau aggregates. Compounds of Formula I or Formula II may be used for the detection of tau aggregates in the diagnosis or monitoring of the progression of a disease or disorder such as Alzheimer's disease, corticobasal degeneration and progressive supranuclear palsy.

### CROSS-REFERENCE TO RELATED APPLICATION

This application claims priority to U.S. Provisional Patent Application No. 63/465,817, filed on May 11, 2023. The contents of this application are incorporated herein by reference in its entirety.

### STATEMENT REGARDING FEDERALLY FUNDED RESEARCH

This invention was made with government support under U19 NS 110456 awarded by the National Institutes of Health. The government has certain rights in the invention.

### BACKGROUND

The development of biomarkers to assess the accumulation of tau protein in the living human brain is crucial to better understand the pathophysiology tauopathies, such as Alzheimer's Disease, progressive supranuclear palsy (PSP) and corticobasal degeneration (CBD). Over the past 20 years, much progress has been made in developing positron emission tomography (PET) imaging biomarkers for AD. While three FDA-approved amyloid-P plaque PET radiopharmaceuticals have become available over the past decade for widespread imaging of amyloid-beta plaques in AD, first and second generation AD tau-PET tracers are still emerging. AD aggregates are comprised of a mixture of both 3R- and 4R-tau, while PSP and CBD tau aggregates are comprised primarily of 4R-tau, and PET imaging agents developed for imaging tau deposits in AD (such as AV-1451, PI-2620, MK-6240, and PM-PBB3) may not bind with high affinity to the tau deposits in PSP and CBD patients. Hence, the development of high affinity 4R-tau selective PET imaging agents is among the biggest challenges in PSP and CBD research.

There is a need in the art for 4R-tau selective binding agents, and the present disclosure satisfies this need.

### SUMMARY

According to one aspect of the present disclosure, a compound having a structure of Formula I or Formula II, or a pharmaceutically acceptable salt, solvate, hydrate, tautomer or stereoisomer thereof, is provided:

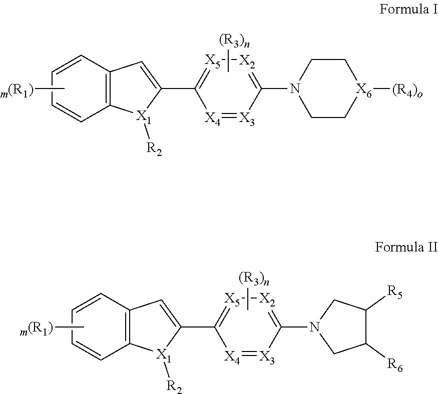

where:
X₁ is N, O or S;
X₂, X₃, X₄, X₅, are each independently N or CH;
X₆ is CH or O;
R₁ is H, halogen, CN, Me₃N⁺X₇⁻, Et₃N⁺X₇⁻, Pr₃N⁺X₇⁻, Bu₃N⁺X₇⁻, or other suitable ammonium salt, OH (with the proviso that when R₁ is OH, R₁ is not substituted at indole ring position 5), OMs, OTs, OTf, O-nosylate, O-brosylate, or other suitable sulfonate species, SnMe₃, SnEt₃, SnPr₃, SnBu₃, or other suitable trialkyltin species, I(O-acyl)₂, ArI⁺X₇⁻, IR₇, S(O)Ar, SO₂Ar, BF₃K, B(OH)₂, B(OMe)₂, B(OEt)₂, B(Opr)₂, B(OiPr)₂, B(OiPr)₃Li, B-pinacol, B-neopentyl glycol, or other suitable boron species; wherein (1) X₇⁻ is a suitable counterion; (2) ArI⁺X₇⁻ is a suitable iodonium species known in the art; and (3) IR₇ is a suitable iodonium ylide known in the art;
R₂ is H, alkyl, tert-butoxycarbonyl, tosyl, benzyl, p-methoxybenzyl, CBz, Fmoc, or other suitable nitrogen protecting group;
R₃ is H, halogen, NO₂, Me₃N⁺X₇⁻, Et₃N⁺X₇⁻, Pr₃N⁺X₇⁻, Bu₃N⁺X₇⁻, or other suitable ammonium salt, OH, OMs, OTs, OTf, O-nosylate, O-brosylate, or other suitable sulfonate species, SnMe₃, SnEt₃, SnPr₃, SnBu₃, or other suitable trialkyltin species, I(O-acyl)₂, ArI⁺X₇⁻, IR₇, S(O)Ar, SO₂Ar, BF₃K, B(OH)₂, B(OMe)₂, B(OEt)₂, B(Opr)₂, B(OiPr)₂, B(OiPr)₃Li, B-pinacol, B-neopentyl glycol, or other suitable boron species; wherein (1) X₇⁻ is a suitable counterion; (2) ArI⁺X₇⁻ is a suitable iodonium species known in the art; and (3) IR₇ is a suitable iodonium ylide known in the art; R₄ is H, halogen, OH, alkoxy, carbonyl, or OEtOEtF; R₅ is H, halogen, OH, OMs, OTs, OTf, O-nosylate, O-brosylate, or other suitable sulfonate species;
R₆ is H, halogen, OH, O-acyl, O-alkyl, OBoc, or other suitable oxygen protecting species; or R₅ and R₆ form an epoxide:

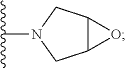 [SMILES: *N1CC2OC2C1]

m and n are each independently 0 or 1; and o is 0, 1, or 2.

In some embodiments, X₁ is N; R₁ is H or halogen; R₂ is H; R₃ is H or halogen; R₄ is alkoxy; R₅ is halogen; R₆ is OH. In some embodiments, the halogen is F, Cl, I, or Br. In some embodiments, the halogen is F.

In some embodiments, the compound has the following structure:

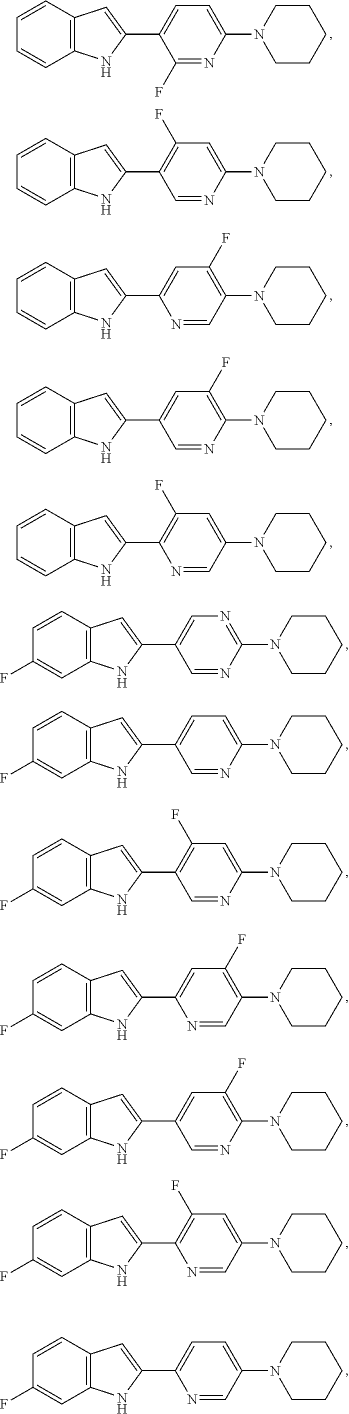

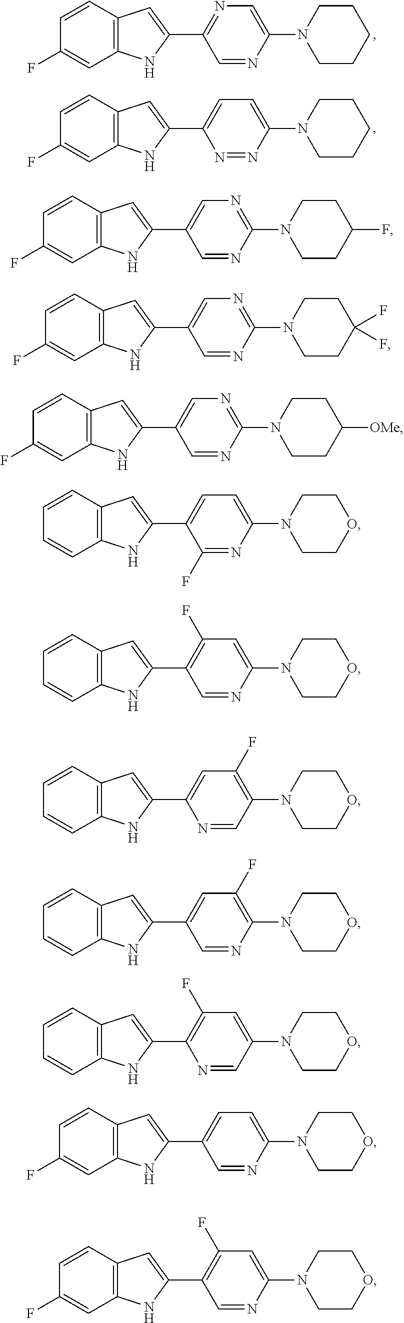

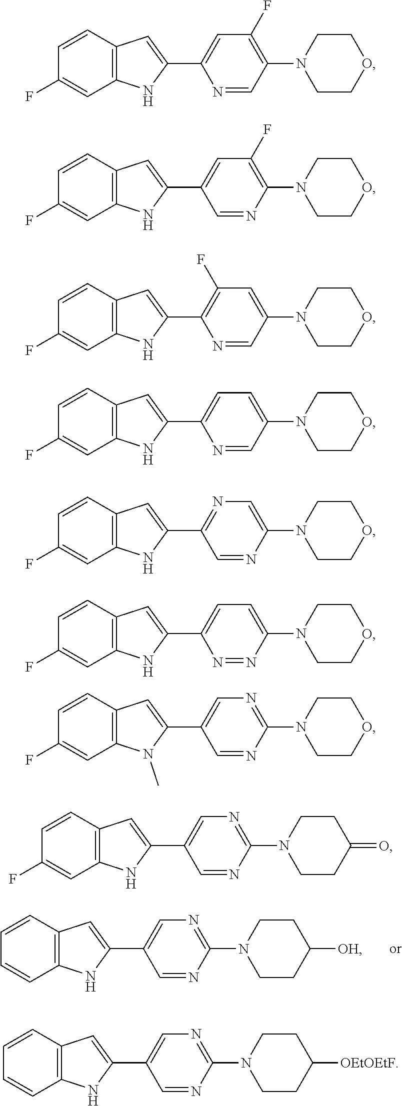

In some embodiments, the compound has the following structure:

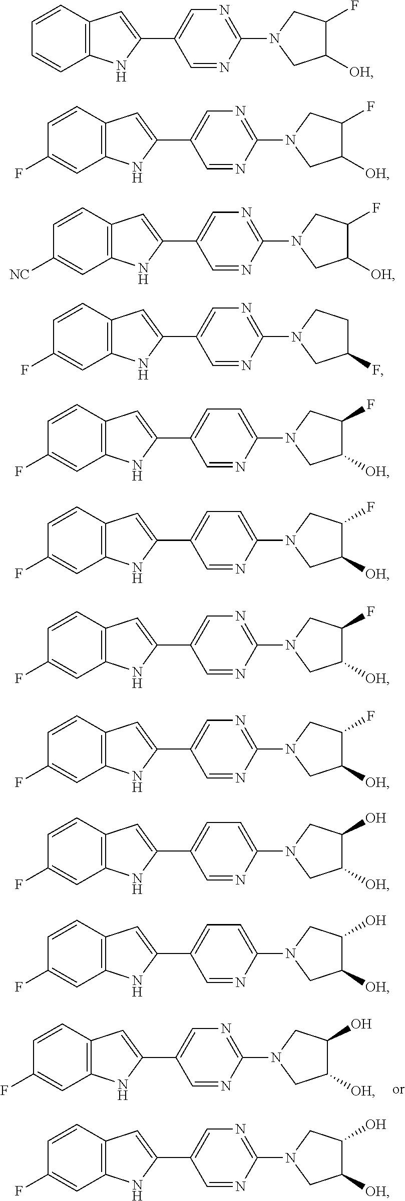

In some embodiments, any one of the compounds described herein selectively binds to 4R tau.

In some embodiments, any one of the compounds described herein comprises one or more detection labels.

In some embodiments, the detection label is selected from the group consisting of a radionuclide, a positron emitter, a gamma emitter, and a fluorescent label.

In some embodiments, the detection label is selected from the group consisting of ²H, ³H, ¹⁸F, ¹²³I, ¹²⁴I, ¹²⁵I, ¹³¹I, ¹¹C, ¹³C, ¹⁴C, ¹³N, ¹⁵N, ¹⁵O, ⁷⁵Br, ⁷⁶Br, and ⁷⁷Br.

In some embodiments, wherein the detection label is ¹⁸F.

In some embodiments, any one of the compounds described herein is for use in detection of a disorder or disease associated with tau aggregates.

In some embodiments, the disease or disorder is a 4R-tauopathy or a disease or disorder correlated with aggregates of 4R tau.

In some embodiments, the disease or disorder is a neurodegenerative disease or disorder.

In some embodiments, the disorder or disease is selected from the group consisting of Alzheimer's disease, progressive supranuclear palsy, Pick's disease, frontal temporal lobar dementias (FTLD's), frontotemporal dementia with parkinsonism-17 (FTDP-17), Primary age-related tauopathy (PART) dementia, argyrophilic grain disease, glial globular tauopathy, chronic traumatic encephalopathy (CTE), corticobasal syndrome (CBS), and corticobasal degeneration.

In some embodiments, detection is carried out carried out by one or more of positron emission tomography (PET) imaging, single photon emission computed tomography (SPECT), magnetic resonance imaging, or autoradiography.

In some embodiments, detection is carried out by positron emission tomography (PET) imaging.

In an aspect, a method for diagnosing a subject with a disease or disorder, or monitoring disease or disorder progression in a subject is provided. The method includes: administering to the subject a detectable quantity of an isotopically labeled compound of Formula I or Formula II, or a pharmaceutically acceptable salt, solvate, hydrate, tautomer or stereoisomer thereof.

In some embodiments, the disease or disorder is associated with tau aggregates.

In some embodiments, the disease or disorder is a 4R-tauopathy or a disease or disorder correlated with aggregates of 4R tau.

In some embodiments, the disease or disorder is a neurodegenerative disease or disorder.

In some embodiments, the disease or disorder is selected from the group consisting of Alzheimer's disease, progressive supranuclear palsy, Pick's disease, frontal temporal lobar dementias (FTLD's), frontotemporal dementia with parkinsonism-17 (FTDP-17), Primary age-related tauopathy (PART) dementia, argyrophilic grain disease, glial globular tauopathy, chronic traumatic encephalopathy (CTE), corticobasal syndrome (CBS), and corticobasal degeneration.

In some embodiments, the compound is isotopically labeled with ²H, ³H, ⁸F, ¹²³I, ¹²⁴I, ¹²⁵I, ¹³¹I, ¹¹C, ¹³C, ¹⁴C, ¹³N, ¹⁵N, ¹⁵O, ⁷⁵Br, ⁷⁶Br, or ⁷⁷Br.

In some embodiments, detection is carried out carried out by one or more of positron emission tomography (PET) imaging, single photon emission computed tomography (SPECT), magnetic resonance imaging, or autoradiography.

In all of the methods described herein, the subject can be a human.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIG. 1 shows the X-ray crystal structure of Compound 38.

FIG. 2 shows the X-ray crystal structure of Compound 65.

FIG. 3 shows the X-ray crystal structure of Compound 66.

FIG. 4 shows the X-ray crystal structure of Compound 69 (JSS20-183A).

FIG. 5 shows brain PET images and brain time-activity curves of [¹⁸F]68 in a male rhesus macaque (12 kg).

FIG. 6 shows brain PET images and brain time-activity curves [¹⁸F]69 ([¹⁸F]JSS20-183A) in a male rhesus macaque (13 kg).

FIG. 7 shows post-mortem medial frontal cortex sections from PSP, CBD, and AD subjects, and cingulate gyrus from a 60-year-old control subject with 5 nM [³H]69 ([³H]JSS20-183A) and self-blocking in adjacent sections with 1 mM unlabeled 69 (JSS20-183A).

### DETAILED DESCRIPTION

### I. Overview

The present invention is directed to the discovery of new compounds that selectively bind to tau, and in particular 4R-tau. The compounds are useful for example in methods of imaging aggregated Tau in tauopathies.

Tau is a protein that helps stabilize the internal skeleton of nerve cells (neurons) in the brain. This internal skeleton has a tube-like shape through which nutrients and other essential substances travel to reach different parts of the neuron. Tauopathies are age-related neurodegenerative diseases that are characterized by the presence of aggregates of abnormally phosphorylated tau. In these neurodegenerative diseases, tau detaches from microtubules to form insoluble aggregates leading to tauopathy.

Six different isoforms of the microtubule-associated protein tau exist in the human adult brain: 0N3R, 0N4R, 1N3R, 1N4R, 2N3R, 2N4R. These isoforms differ in the number of N-terminal inserts (0N, 1N, 2N) and C-terminal repeat domains (3R or 4R) and are differentially expressed depending on the brain region and developmental stage. Human adult tau has approximately equal representation of 3R and 4R tau isoforms, with the 1N3R and 1N4R being the most abundant forms.

As used herein, “tau” refers to a highly soluble microtubule binding protein mostly found in neurons and includes the major 6 isoforms, cleaved or truncated forms, and other modified forms such as arising from phosphorylation, glycosylation, glycation, prolyl isomerization, nitration, acetylation, polyamination, ubiquitihation, sumoylation and oxidation.

Although all tau isoforms can aggregate and form neurofibrillary tangles, some tauopathies, such as Pick's disease and progressive supranuclear palsy, are characterized by the accumulation of specific tau isoforms. AD aggregates are comprised of a mixture of both 3R- and 4R-tau, while PSP and CBD tau aggregates are comprised primarily of 4R-tau (Buee L; Delacourte A, “Comparative biochemistry of tau in progressive supranuclear palsy, corticobasal degeneration, FTDP-17 and Pick's disease,” Brain Pathology 1999, 9 (4), 681-693)). Expression of the 3R isoform causes more profound axonal transport defects and locomotor impairments, culminating in a shorter lifespan than the 4R isoform. In contrast, the 4R isoform leads to greater neurodegeneration and impairments in learning and memory. Tau isoform expression is directly linked to brain development: during neurogenesis, only the shortest TAU isoform, 0N3R, is expressed, whereas in the adult brain, all six isoforms are present with roughly equal amounts of 3R and 4R isoforms.

Diagnosis of 4R tau specific neurological diseases has a number of benefits. The development of biomarkers to assess the accumulation of tau protein in the living human brain is crucial to better understand the pathophysiology of AD, as well as non-Alzheimer's Disease (non-AD) tauopathies, such as progressive supranuclear palsy (PSP) and corticobasal degeneration (CBD). 4R-Tau biomarkers can aid clinicians in making more definitive disease diagnoses as well as confirm target engagement and help determine dosing regimens of new anti-tau therapies currently under development.

Over the past 20 years, much progress has been made in developing positron emission tomography (PET) imaging biomarkers for AD. While several FDA-approved amyloid-P plaque PET radiopharmaceuticals have become available over the past decade for widespread imaging of amyloid-beta plaques in AD (AV-1451, PI-2620, MK-6240, and PM-PBB3), first and second generation AD tau-PET tracers are still emerging. AD aggregates are comprised of a mixture of both 3R- and 4R-tau, while PSP and CBD tau aggregates are comprised primarily of 4R-tau, and PET imaging agents developed for imaging tau deposits in AD (AV-1451, PI-2620, MK-6240, PM-PBB3) may not bind with high affinity to the tau deposits in PSP and CBD patients. Hence, the development of high affinity 4R-tau selective PET imaging agents is among the biggest challenges in PSP and CBD research (Betthauser T J., “In vitro evidence for a nonselective 4R tau PET tracer,” Mol Psychiatry. 2023 Jan. 19. doi: 10.1038/s41380-023-01950-2. Epub ahead of print). For example, PET imaging studies have been carried out in PSP and CBD patients with [¹⁸F]PI-2620 and [¹⁸F]APN-1607. (Li et al., “Progressive Supranuclear Palsy Neuroimage Initiative (PSPNI). Clinical Utility of 18F-APN-1607 Tau PET Imaging in Patients with Progressive Supranuclear Palsy,” Mov Disord. 2021 October; 36(10):2314-2323; Tagai et al., “An optimized reference tissue method for quantification of tau protein depositions in diverse neurodegenerative disorders by PET with 18F-PM-PBB3 (18F-APN-1607),” Neuroimage. 2022 Dec. 1; 264:119763).

Previously reported tau ligands include the following:

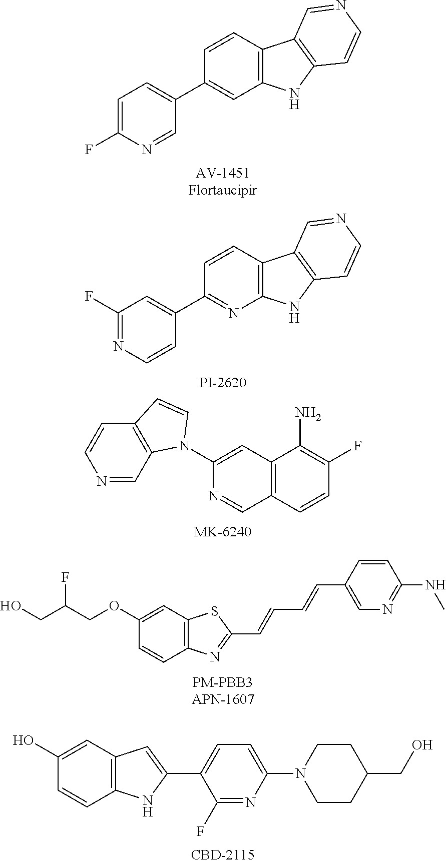

¹⁸F-flortaucipir (AV-1451) is a tau PET ligand which binds to paired helical filaments of tau in aging and AD, but it's utility in detecting aggregates in frontotemporal dementia is uncertain (Tsai et al., ¹⁸F-flortaucipir (AV-1451) tau PET in frontotemporal dementia syndromes,” Alzheimer 's Research and Therapy, 11(13) (2019)). PI-2620 and MK-6240 are second generation tau PET tracers (Malarte et al., “Discriminative binding of tau PET tracers P12620, MK6240, and RO948 in Alzheimer's Disease, corticobasal degeneration and progressive supranuclear palsy brains,” Molecular Psychiatry, 28:1272-1283 (2023)), as well as PM-PBB3 and CBD-2115 (Zhou et al., “Dissecting the Binding Profile of PET Tracers to Corticobasal Degeneration Tau Fibrils,” ACS Chem. Neurosci., 12:3487-3496 (2021)).

However, the in vivo specific signals from the existing 4R-tau tracers are quite low, and improvement in the binding affinity of 4R-tau PET tracers likely is needed to improve the 4R-tau biomarker imaging signals in PSP and CBD patients. The present invention surprisingly and unexpectedly addresses this problem present in the prior art.

In one aspect of the disclosure, the described compounds of Formula I and II selectively bind to 4R tau as defined by whether the compound binds to other protein binding sites (e.g., aggregated amyloid-beta and aggregated alpha-synuclein). The binding affinity (K_{d} or Kᵢ) of the compounds was determined at other protein binding sites, and a K_{d} and/or Kᵢ value greater than about 50 nm at the other binding sites indicates that the compound does not display significant in vivo specific imaging signal.

Non-limiting examples of the disorder or disease associated with tau aggregates, including for example 4R-tau aggregates, include Alzheimer's disease, progressive supranuclear palsy, Pick's disease, frontal temporal lobar dementias (FTLD's), frontotemporal dementia with parkinsonism-17 (FTDP-17), Primary age-related tauopathy (PART) dementia, argyrophilic grain disease, glial globular tauopathy, chronic traumatic encephalopathy (CTE), corticobasal syndrome (CBS), and corticobasal degeneration.

### II. Experimental Results

In an exemplary aspect, Enamine Z3777013540 (Z-3540 or Compound 1) was identified through a molecular similarity fingerprint search of the Enamine compound library based on CBD-2115. Z-3540 is an exemplary compound according to the invention. [3H]Z-3540 was synthesized and evaluated in binding assays in human CBD, PSP and AD tissues.

### III. Compounds

Provided herein are heterocyclic compounds for imaging aggregated tau in tauopathies. To date, no high-affinity 4R-tau selective PET tracer suitable for neuroimaging exists.

The heterocyclic compounds described herein have a structure according to Formula I or Formula II, or a pharmaceutically acceptable salt, solvate, hydrate, tautomer or stereoisomer thereof:

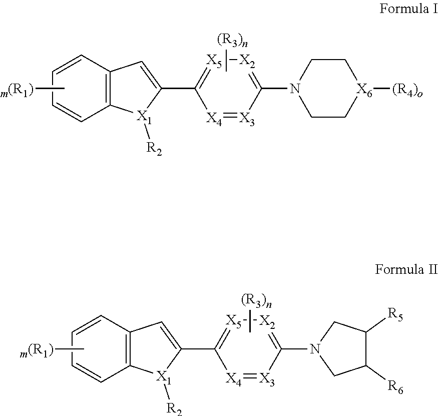

where:
   X₁ is N, O or S;
   X₂, X₃, X₄, X₅, are each independently N or CH;
   X₆ is CH or O;
R₁ is H, halogen, CN, Me₃N⁺X₇⁻, Et₃N⁺X₇⁻, Pr₃N⁺X₇⁻, Bu₃N⁺X₇⁻, or other suitable ammonium salt, OH (with the proviso that when R₁ is OH, R₁ is not substituted at indole ring position 5), OMs, OTs, OTf, O-nosylate, O-brosylate, or other suitable sulfonate species, SnMe₃, SnEt₃, SnPr₃, SnBu₃, or other suitable trialkyltin species, I(O-acyl)₂, ArI⁺X₇⁻, IR₇, S(O)Ar, SO₂Ar, BF₃K, B(OH)₂, B(OMe)₂, B(OEt)₂, B(Opr)₂, B(OiPr)₂, B(OiPr)₃Li, B-pinacol, B-neopentyl glycol, or other suitable boron species; wherein (1) X₇⁻ is a suitable counterion; (2) ArI⁺X₇⁻ is a suitable iodonium species known in the art; and (3) IR₇ is a suitable iodonium ylide known in the art;
   R₂ is H, alkyl, tert-butoxycarbonyl, tosyl, benzyl, p-methoxybenzyl, CBz, Fmoc, or other suitable nitrogen protecting group;
   R₃ is H, halogen, NO₂, Me₃N⁺X₇⁻, Et₃N⁺X₇⁻, Pr₃N⁺X₇⁻, Bu₃N⁺X₇⁻, or other suitable ammonium salt, OH, OMs, OTs, OTf, O-nosylate, O-brosylate, or other suitable sulfonate species, SnMe₃, SnEt₃, SnPr₃, SnBu₃, or other suitable trialkyltin species, I(O-acyl)₂, ArI⁺X₇⁻, IR₇, S(O)Ar, SO₂Ar, BF₃K, B(OH)₂, B(OMe)₂, B(OEt)₂, B(Opr)₂, B(OiPr)₂, B(OiPr)₃Li, B-pinacol, B-neopentyl glycol, or other suitable boron species; wherein (1) X₇⁻ is a suitable counterion; (2) ArI⁺X₇⁻ is a suitable iodonium species known in the art; and (3) IR₇ is a suitable iodonium ylide known in the art;
   R₄ is H, halogen, OH, alkoxy, carbonyl, or OEtOEtF;
   R₅ is H, halogen, OH, OMs, OTs, OTf, O-nosylate, O-brosylate, or other suitable sulfonate species;
   R₆ is H, halogen, OH, O-acyl, O-alkyl, OBoc, or other suitable oxygen protecting species; or Rs and R₆ form an epoxide:

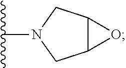 [SMILES: *N1CC2OC2C1]

m and n are each independently 0 or 1; and o is 0, 1, or 2.

In some embodiments, X₁ is N; R₁ is H or halogen; R₂ is H; R₃ is H or halogen; R₄ is alkoxy; R₅ is halogen; R₆ is OH. In some embodiments, the halogen is F, Cl, I, or Br. In some embodiments, the halogen is F.

The compound of Formula I may have one of the following structures:

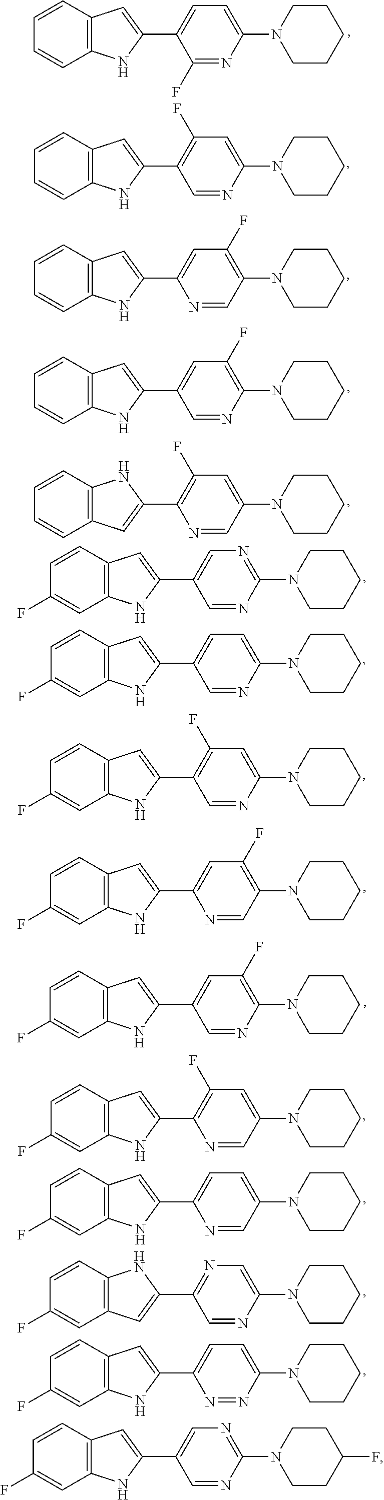

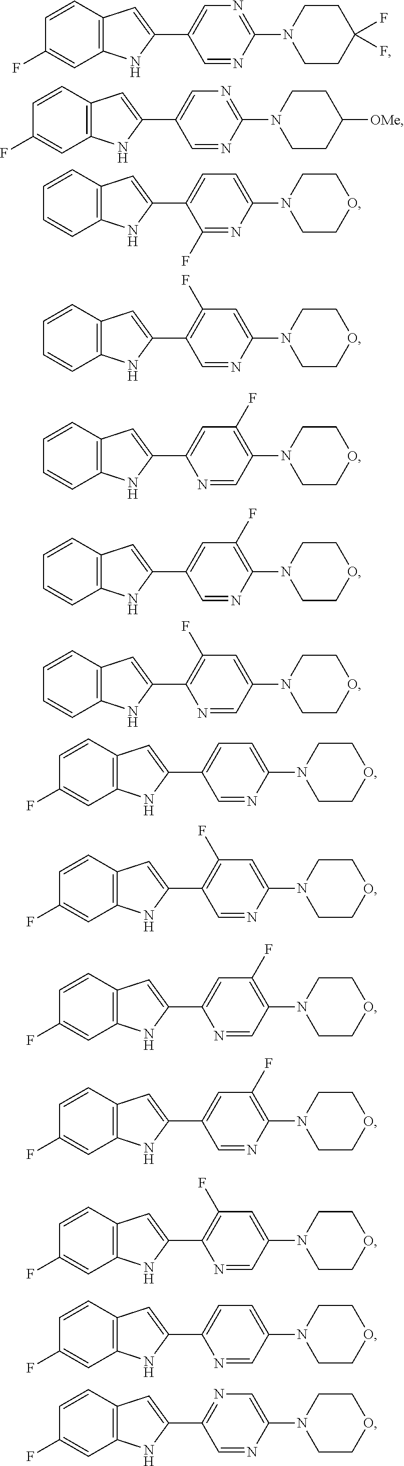

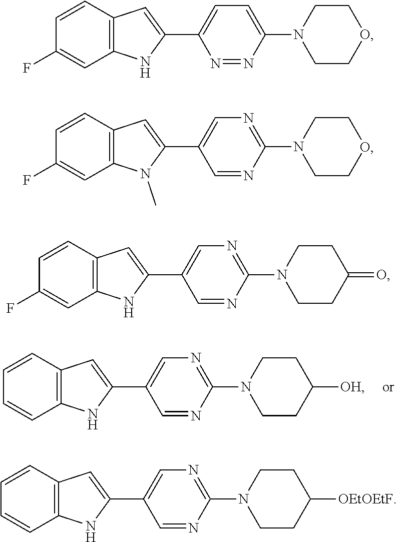

The compound of Formula 11 may have one of the following structures:

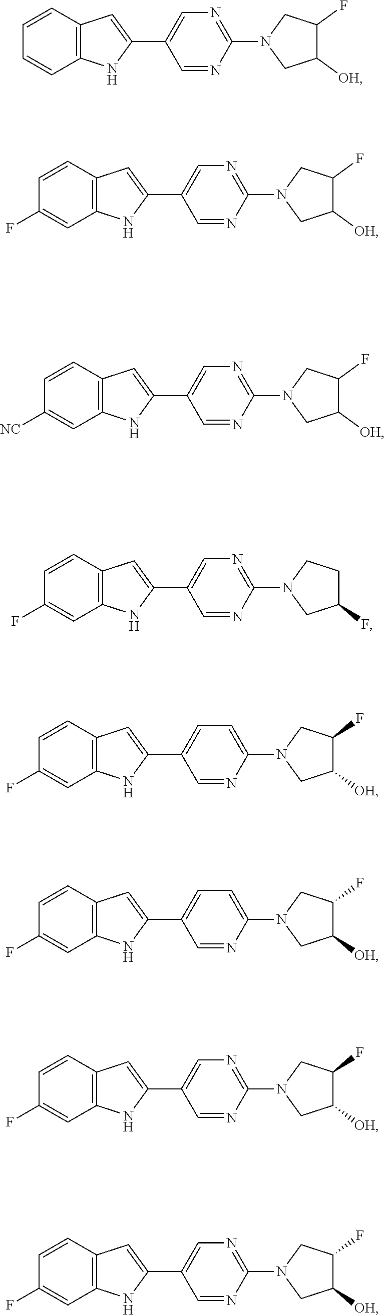

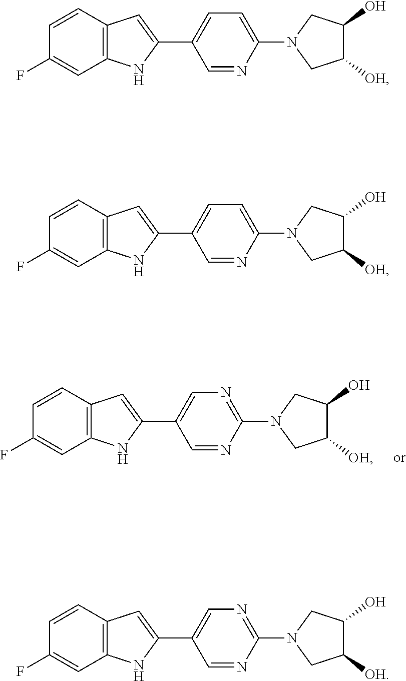 [SMILES: O[C@@H]1CN(c2ccc(-c3cc4ccc(F)cc4[nH]3)cn2)C[C@H]1O ; O[C@H]1CN(c2ccc(-c3cc4ccc(F)cc4[nH]3)cn2)C[C@@H]1O ; O[C@@H]1CN(c2ncc(-c3cc4ccc(F)cc4[nH]3)cn2)C[C@H]1O ; O[C@H]1CN(c2ncc(-c3cc4ccc(F)cc4[nH]3)cn2)C[C@@H]1O]

In some embodiments, the compound of Formula I has one of the following structures:

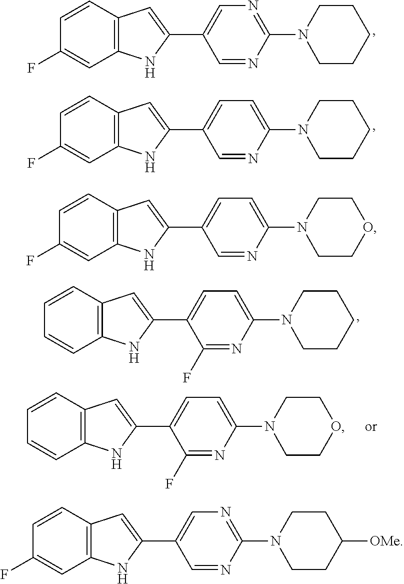

In some embodiments, the compound of Formula II has the following structure:

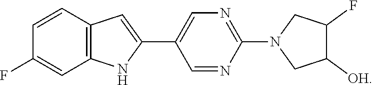 [SMILES: OC1CN(c2ncc(-c3cc4ccc(F)cc4[nH]3)cn2)CC1F]

The salts of the compounds of Formula I or Formula II may be pharmaceutically acceptable salts. Other salts may be useful in the preparation of the compounds according to the disclosure or of their pharmaceutically acceptable salts.

When the compound of the present disclosure is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

An “amine protecting group” is any protecting group which suitable for protecting an amine group during an envisaged chemical reaction. Examples of suitable protecting groups are well-known to a person skilled in the art. Examples thereof include but are not limited to carbamates, amides, imides, N-alkyl amines, N-aryl amines, imines and enamines. Suitable protecting groups are discussed, e.g., in the textbook Greene and Wuts, Protecting groups in Organic Synthesis, third edition, page 494-653, which is included herein by reference. Specific preferred examples of the amine-protecting groups include tert-butyloxycarbonyl (Boc), trimethylsilylethoxycarbonyl (Teoc), carbobenzyloxy (Cbz), p-methoxybenzyl carbonyl (Moz or MeOZ), 9-fluorenylmethyloxycarbonyl (Fmoc), acetyl (Ac), or trifluoroacetyl.

Compounds of Formula I or Formula II may be labelled. A “label” (which may be a radiolabel or other detectable label, or a tag, marker, detectable marker, tracer, radiotracer or equivalent) is any atom or group suitable for imaging and/or assaying (for example, identifying, imaging, diagnosing, evaluating, detecting and/or quantitating) in vivo or in vitro, and in particular imaging and diagnosing. Suitable labels include, for example, radioisotopes (which may also be referred to as “radiolabeled atoms”), radionuclides, isotopes, positron emitters, gamma emitters, fluorescent groups, luminescent groups, chromogenic groups, biotin (in conjunction with streptavidin complexation) or photoaffinity groups. The type of label chosen will depend on the desired detection method. The position at which the label is integrated or attached to the compounds of the present invention is not particularly limited.

Examples of isotopes (such as radioisotopes, radionuclides, positron emitters and gamma emitters) which may be used to label compounds of the invention, include but are not limited to: ²H, ³H, ¹⁸F, ¹²³I, ¹²⁴I, ¹²⁵I, ¹³¹I, ¹¹C, ¹³C, ¹⁴C, ¹³N, ¹⁵N, ¹⁵O, ⁷⁵Br, ⁷⁶Br, and ⁷⁷Br. In some embodiments, the detection label is ¹⁸F.

In some embodiments, any one of the compounds disclosed herein comprises one or more detection labels. In some embodiments, the R₁ and R₃ substituents are detectably labeled. In some embodiments, in any one of the compounds described herein, one or more H, one or more C, one or more N, one or more O, one or more F, one or more Br, and/or one or I may be replaced with a ³H; ¹¹C, ¹³C or ¹⁴C; ¹³N; ¹⁵O; ¹⁸F; ⁷⁵Br or ⁷⁶Br; or ¹²³I, ¹²⁴I, ¹²⁵I, ¹³¹I. In some embodiments, the detection label is ¹⁸F.

In some embodiments, the compounds of the present disclosure selectively bind to 4R-tau. In some embodiments, the compounds of the present disclosure can bind 4R-tau aggregates both in vivo and in vitro. In some embodiments, the compounds of the present disclosure may be for use as a diagnostic agent (for in vivo and/or in vitro diagnostic use) in the diagnosis of disease or disorder associated with 4R-tau aggregates (i.e., tauopathies). In some embodiments, the compounds of the present disclosure are for use in detection of a disorder or disease associated with tau aggregates. In some embodiments, the disease or disorder is a 4R-tauopathy or a disease or disorder correlated with aggregates of 4R tau. In some embodiments, the disease or disorder is a neurodegenerative disease or disorder.

### IV. Methods

Also provided herein are methods for diagnosing a subject with a disease or disorder, or monitoring disease or disorder progression in a subject. In one aspect, the disease or disorder is a neurological disease characterized by the accumulation for 4R tau aggregates in the subject.

In another aspect, encompassed is a method of diagnosing a subject with a non-AD taupathy characterized by an accumulation of 4R tau aggregates in the subject.

In yet another aspect, encompassed is a method of diagnosing a subject with a disease or disorder which is not AD, e.g., eliminating the diagnosis of AD.

In one aspect, encompassed is a method of diagnosing a subject with a neurological disease characterized by the accumulation for 4R tau aggregates in the subject, followed by administering a treatment for the disease or disorder. The disease or disorder can be, for example, progressive supranuclear palsy, Pick's disease, frontal temporal lobar dementias (FTLD's), frontotemporal dementia with parkinsonism-17 (FTDP-17), Primary age-related tauopathy (PART) dementia, argyrophilic grain disease, glial globular tauopathy, chronic traumatic encephalopathy (CTE), corticobasal syndrome (CBS), or corticobasal degeneration

The methods of the disclosure can comprise (a) administering to a subject in need a detectable quantity of an isotopically labeled compound of Formula I or Formula II, or a pharmaceutically acceptable salt, solvate, hydrate, tautomer or stereoisomer thereof, and (b) detecting binding of the compound to tau aggregates in the patient, and in particular binding to 4R-tau aggregates present in the patient.

In some embodiments, detection is carried out carried out by one or more of positron emission tomography (PET) imaging, single photon emission computed tomography (SPECT), magnetic resonance imaging, or autoradiography. In some embodiments, detection is carried out by positron emission tomography (PET) imaging.

### V. Definitions

As used herein, the term “alkyl” refers to both straight and branched chain saturated hydrocarbon groups. Examples of alkyl groups include methyl, ethyl, n-propyl, iso-propyl, n-butyl, t-butyl, i-butyl, sec-butyl, pentyl and hexyl groups. Examples of unbranched alkyl groups include, but are not limited to, methyl, ethyl, n-propyl, n-butyl groups. Examples of branched alkyl groups include, but are not limited to, isopropyl, tertbutyl, isobutyl, 1-ethylpropyl and 1-ethylbutyl groups.

As used herein, the term “halogen” or “halo” refers to fluorine (F), chlorine (Cl), bromine (Br), or iodine (I).

As used herein, the term “alkoxy” refers to an alkyl group of indicated number of carbon atoms attached through an oxygen bridge.

As used herein, the term “heterocyclyl”, “heterocycle” or “heterocyclic” refers to a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. The term heterocyclyl, heterocycle or heterocyclic includes heteroaryl moieties.

As used herein, “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The present invention, thus generally described, will be understood more readily by reference to the following examples, which are provided by way of illustration and are not intended to be limiting of the present invention.

### EXAMPLES

### Example 1. Identification of Compounds that have High Affinity for Aggregated 4R-Tau

The purpose of this example was to identify compounds that have a high affinity for aggregated 4R-tau.

Three 6-fluoroindole-based compounds 1-3 (shown below) available from a commercial source (Enamine) were identified as having high affinity for aggregated 4R-tau in human brain tissue samples from AD, PSP, and CBD patients (Table 1).

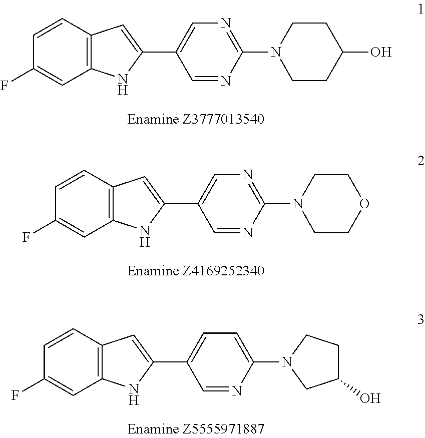 [SMILES: OC1CCN(c2ncc(-c3cc4ccc(F)cc4[nH]3)cn2)CC1 ; Fc1ccc2cc(-c3cnc(N4CCOCC4)nc3)[nH]c2c1 ; O[C@H]1CCN(c2ccc(-c3cc4ccc(F)cc4[nH]3)cn2)C1]

TABLE 1
Dissociation constants of [³H]1
and [³H]2 in human brain tissue samples.

| | K_{D} (nM) | | |
|---|---|---|---|
| Compound | AD brain | PSP brain | CBD brain |
| [³H]1 | 4.0 ± 3.1 | 5.1 ± 1.2 | 4.1 |
| [³H]2 | 1.2 ± 0.5 | 1.6 ± 0.2 | 1.7 ± 0.3 |

These compounds were identified through the combination of ultrahigh throughput in silico screening and in vitro binding assays for “hit confirmation”. Subsequent chemical synthesis and structure-activity relationship studies identified numerous 4R-tau ligands (shown below) which display high affinity in human brain tissue samples from AD, PSP, and CBD patients (Table 2 below).

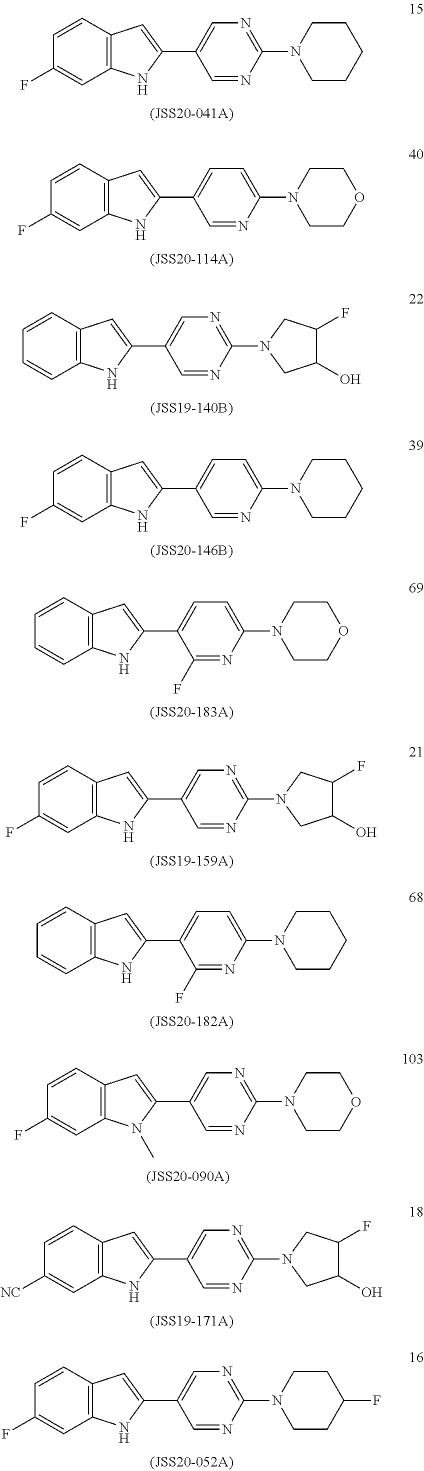

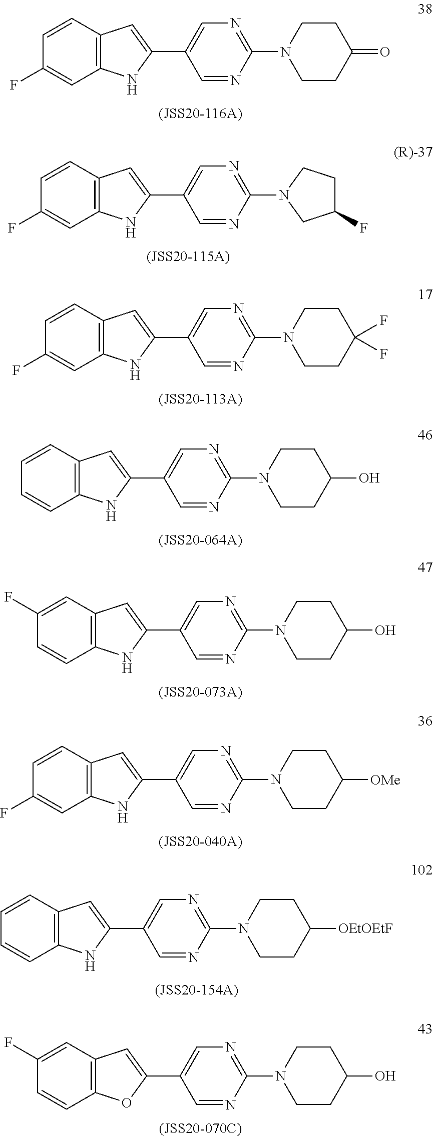

### Example 2. Synthesis of 4R-Tau Ligands

The purpose of this example was to synthesize various 4R-tau ligands.

NMR spectra were obtained on Bruker Avance III spectrometers at the specified frequencies. Spectra in CDCl₃ are referenced to internal TMS, spectra in other deuterated solvents are referenced to solvent residual protons.

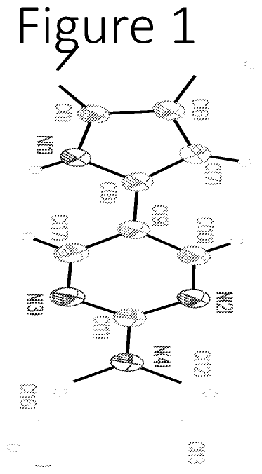

Compounds 4 and 5 (Scheme 1) were prepared according to a previously described procedure (Kumar, A., et al.; Synthesis 2008, 707-710); while compound 6 is commercially available. Compounds 4-6 were coupled to 5-bromo-2-chloropyrimidine under Pd-catalysis to afford compounds 7-11, with N-Boc-protected (7, 9, and 11) and N-deprotected (8 and 10) compounds all being isolated. Compound 7 was reacted with either piperidine, 4-fluoropiperidine hydrochloride, or 4,4-difluoropiperidine hydrochloride to afford compounds 12, 13, and 14, respectively, followed by deprotection with TFA to give 15, 16, and 17, respectively. Compound 10, and Boc-protected compounds 7 and 11, were reacted with trans/racemic-4-fluoro-3-hydroxypyrrolidine hydrochloride to afford compounds 18, 19, and 20, respectively, then the N-Boc protecting groups of 19 and 20 were removed with TFA to afford compounds 21 and 22, respectively. 5-Bromo-2-chloropyrimidine was reacted with various cyclic amines (Scheme 2) to give compounds 23, 24, (R)-25, and 26. Compound 26 was then oxidized with Dess-Martin periodinane to give compound 27. 2,5-Dibromopyridine was reacted with either piperidine or morpholine (Scheme 3) to give compounds 28 and 29, respectively. 2,5-Dibromopyrazine was reacted with morpholine (Scheme 3) to give compound 30. Pd-catalyzed coupling of 4 (Scheme 4) with compounds 23, (R)-25, 27, 28, and 29 gave N-Boc-protected compounds 31, (R)-32, 33, 34, and 35, respectively, along with the N-deprotected compound 36. Compounds 31-35 were then deprotected with TFA to afford compounds 36, (R)-37, 38, 39, and 40 (the structure of 38 was confirmed by X-ray crystallography, FIG. 1). Commercially available compounds 41, 6, and 42 (Scheme 5) were coupled to compound 26 to give compounds 43, 44, and 45, respectively. Compounds 44 and 45 were then deprotected with TFA to afford compounds 46 and 47, respectively.

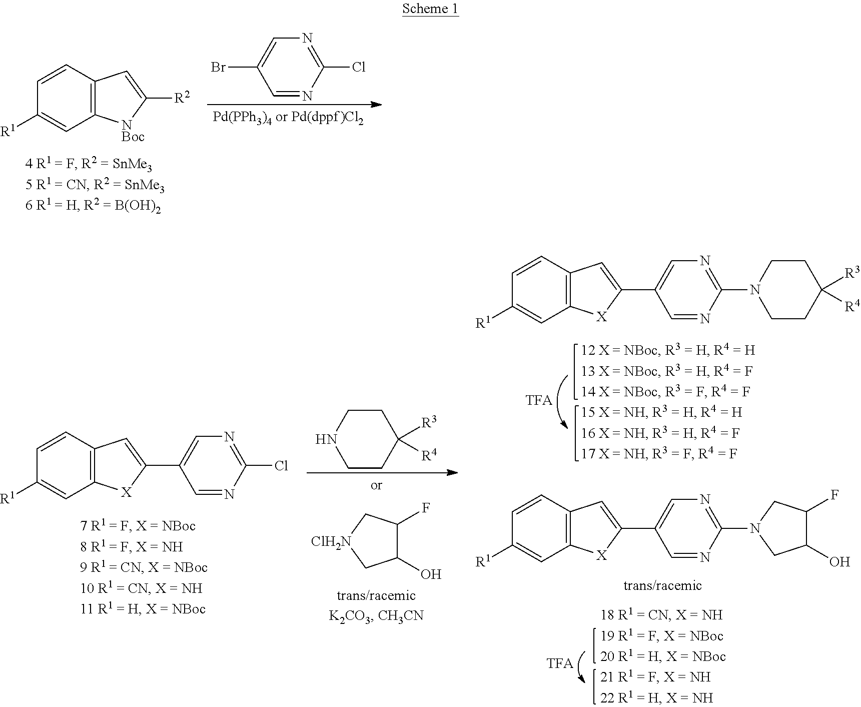

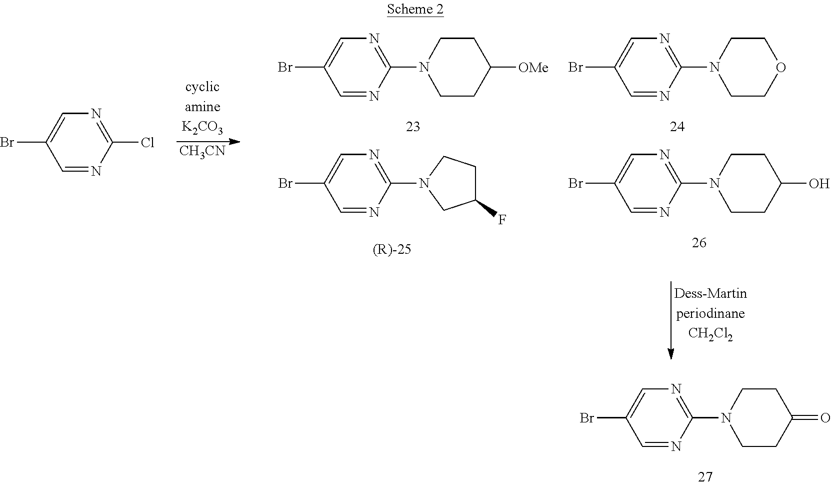

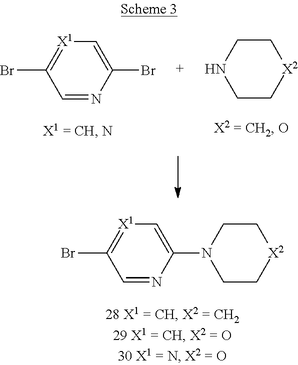

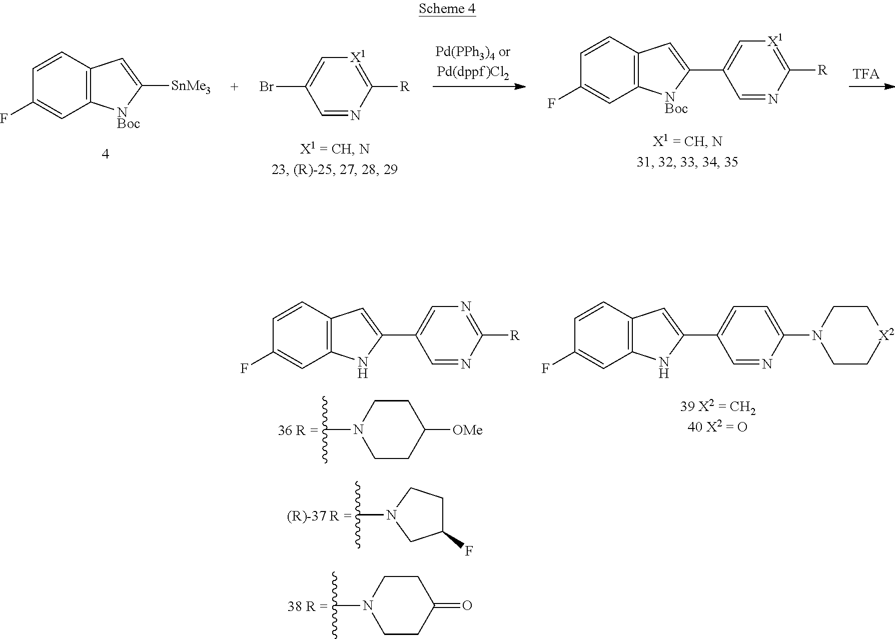

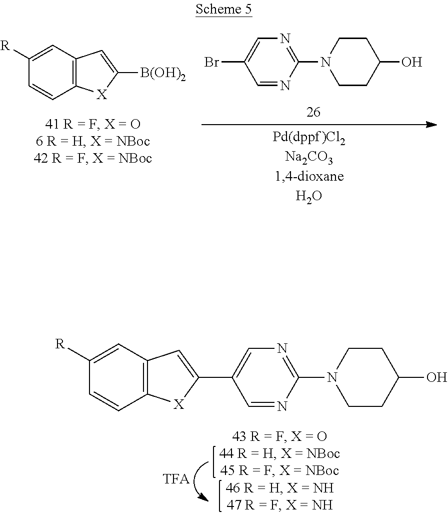

2,6-Difluoropyridine and 2-chloro-6-nitropyridine were each reacted with piperidine or morpholine (Scheme 6) to give compounds 48, 49, 50, and 51, followed by bromination with NBS to give compounds 52, 53, 54, and 55 (Smith, A. C., et al, Organic Process Research & Development 2018, 22, 681-696). 2-Bromo-4-fluoropyridine was reacted with morpholine (Scheme 7) to give compound 56 which was then brominated to give compound 57. 2,5-Dibromo-3-fluoropyridine, 5-bromo-2-chloro-3-fluoropyridine, and 5-bromo-2-chloro-4-fluoropyridine (Scheme 8) were each coupled to either piperidine or morpholine under Pd-catalysis (Ji, J., et al, Organic Letters 2003, 5, 4611-4614; Stroup, B. W., et al, Organic Letters 2007, 9, 2039-2042) to give compounds 58, 59, 60, 61, and 62.

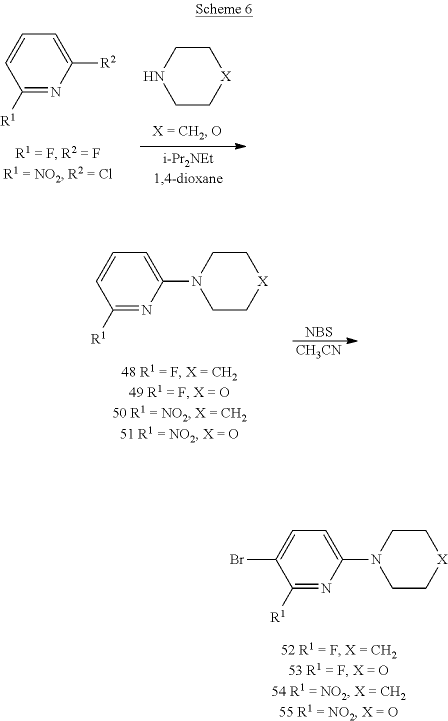

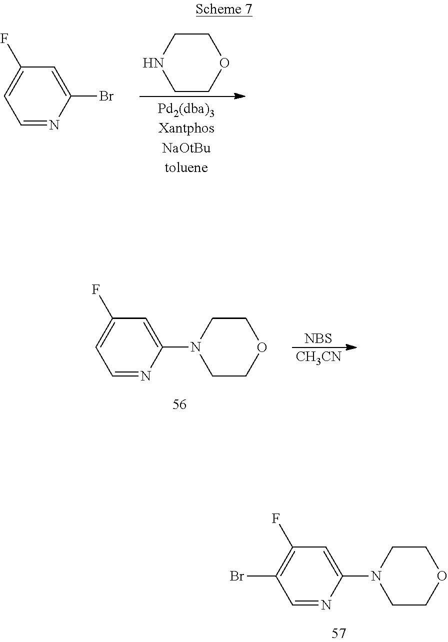

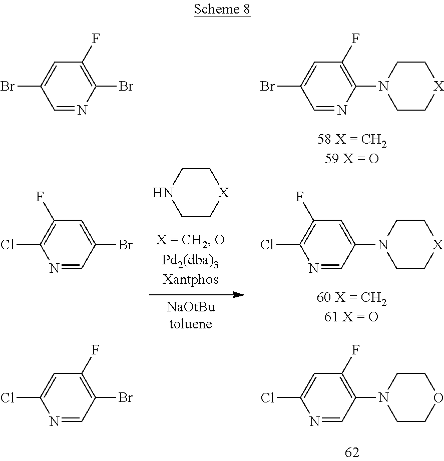

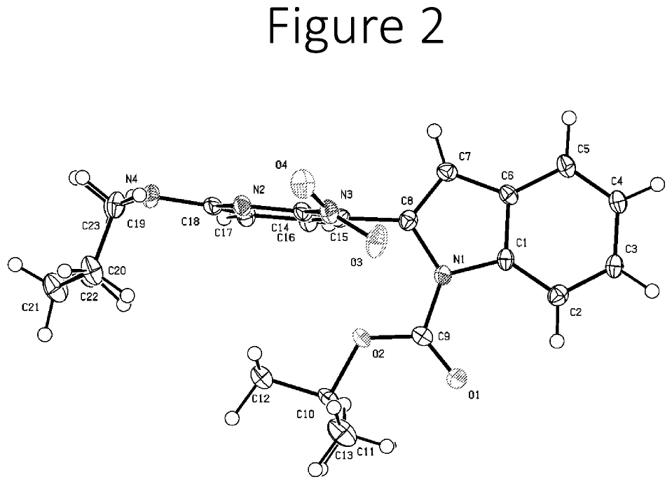

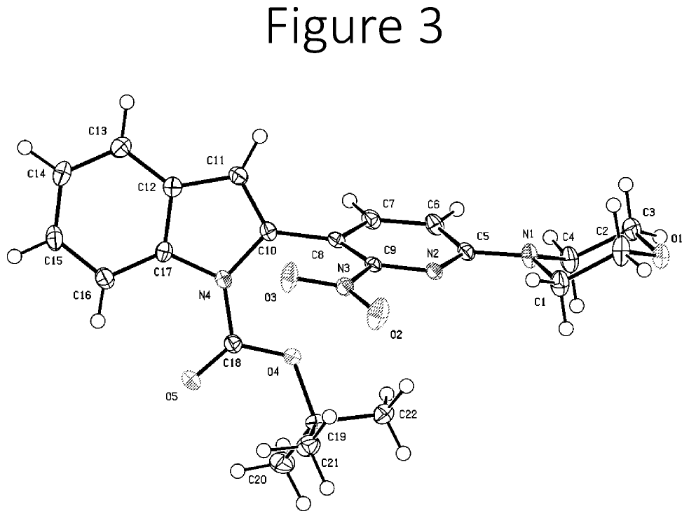

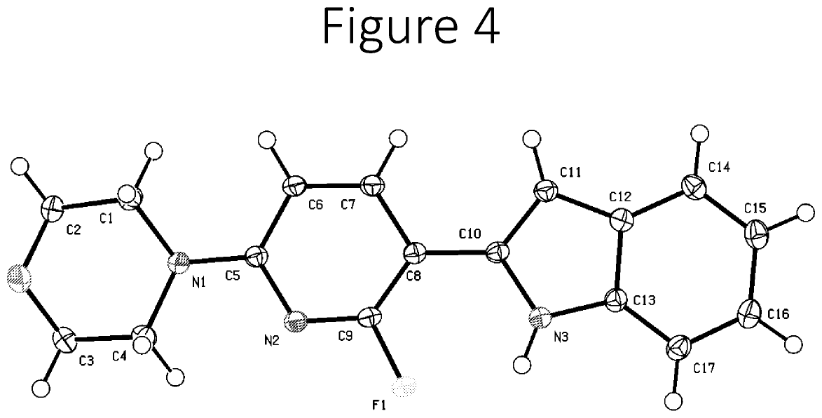

Compounds 52-55 were coupled to compound 6 under Pd-catalysis (Scheme 9) to give N-Boc protected compounds 63, 64, 65, and 66, respectively, along with the N-deprotected compound 67. Compounds 63 and 64 were then deprotected with TFA to give compounds 68 and 69 (JSS20-183A), respectively. X-ray crystallography confirmed the structures of Compound 65 (FIG. 2), Compound 66 (FIG. 3), and Compound 69 (FIG. 4).

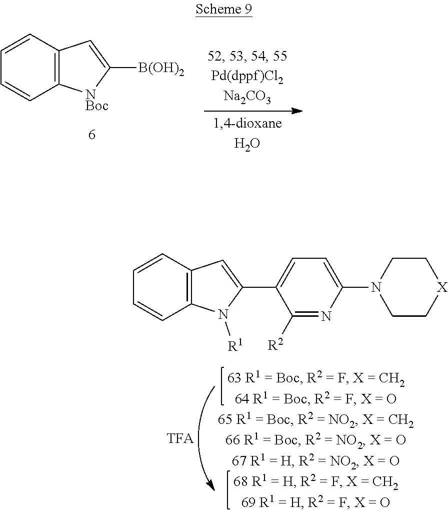

Compound 42 was coupled to compounds 24 and 29 (Scheme 10) to give compounds 70 and 71, respectively, which were then deprotected with TFA to afford compounds 72 and 73, respectively. Compound 4 was coupled to 52 (Scheme 11) to give compound 74 which was then deprotected with TFA to give compound 75. Compound 76 was coupled to compounds 30 and 57-59 (Scheme 11) to give compounds 77, 78, 79, and 80, respectively, which were then deprotected with TFA to give compounds 81, 82, 83, and 84, respectively. Compound 6 was coupled to compounds 57-59 (Scheme 11) to give compounds 85, 86, and 87, respectively, which were then deprotected with TFA to give compounds 88, 89, and 90, respectively.

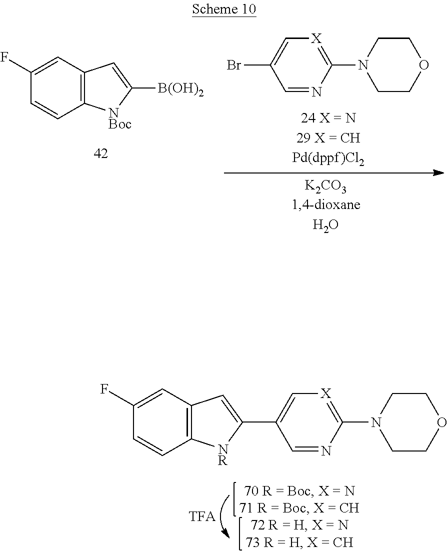

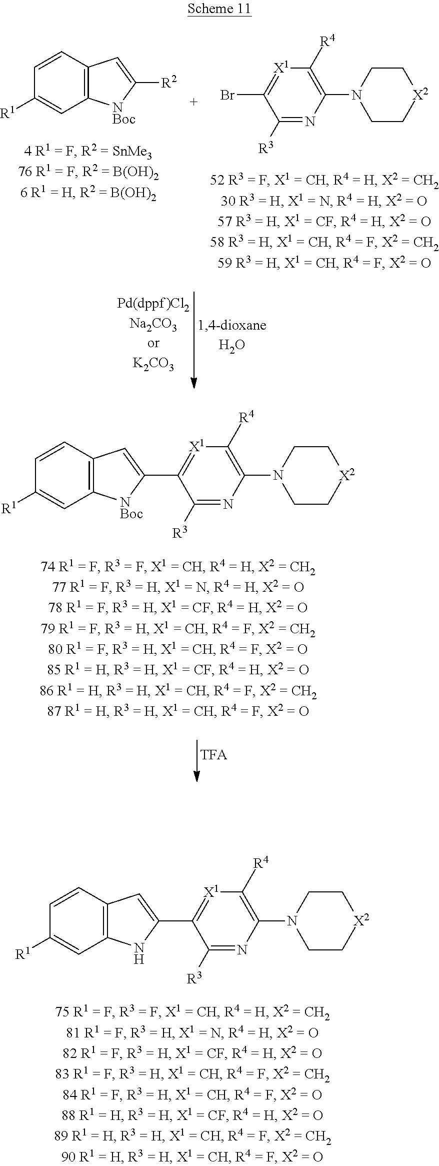

Compound 6 was coupled to compound 60 (Scheme 12) to give compound 91 which was then deprotected with TFA to give compound 92. Compounds 6 and 76 were each coupled to compounds 61 and 62 (Scheme 12) to give compounds 93, 94, 95, and 96, respectively, which were then deprotected with TFA to give compounds 97, 98, 99, and 100, respectively.

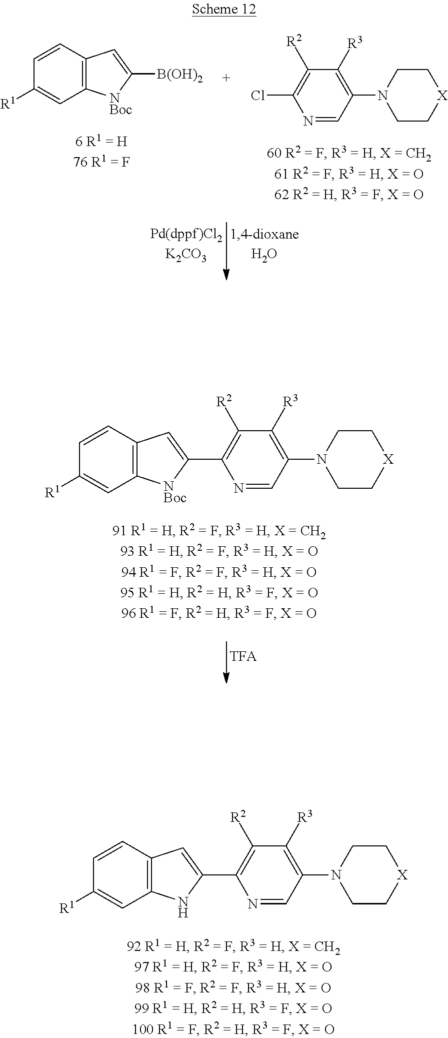

Compound 44 was deprotonated with NaH (Scheme 13), then O-alkylated with 2-(2-fluoroethoxy)ethyl 4-methylbenzenesulfonate to give compound 101 which was then deprotected with TFA to give compound 102. Compound 2 was deprotonated with NaH (Scheme 14), then N-methylated with methyl tosylate to afford compound 103.

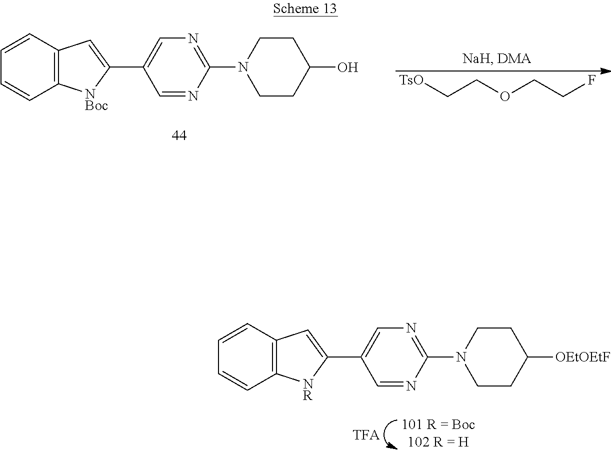

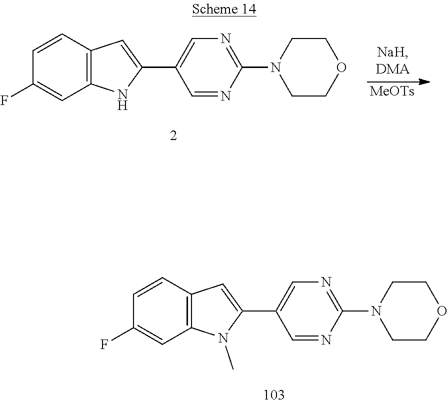 [SMILES: "MeOTs" ; Fc1ccc2cc(-c3cnc(N4CCOCC4)nc3)[nH]c2c1 ; Cn1c(-c2cnc(N3CCOCC3)nc2)cc2ccc(F)cc21]

### Methods

### General

Solvents and reagents were used as received. NMR spectra were obtained on Bruker Avance III spectrometers at the specified frequencies. ¹H NMR spectra in CDCl₃ are referenced to internal TMS, whereas spectra in DMSO-d6, CD30D, or acetone-d6 are referenced to solvent residual protons. ¹³C NMR spectra are referenced to solvent resonances. ¹⁹F NMR spectra are unreferenced. Radial chromatography was performed on a Harrison Research Chromatotron™ using silica rotors from Miles Scientific. Silica gel used was Silicycle SiliaFlash® P60 40-63 μm (230-400 mesh).

### Chemistry

### tert-Butyl 6-fluoro-2-(trimethylstannyl)-1H-indole-1-carboxylate (Compound 4)

Compound 104 (2.23 g, 9.48 mmol) and Me₃SnCl (2.09 g, 10.49 mmol, 1.1 equiv.) were flushed with N_{2(g)} for 40 min, then dissolved in freshly distilled THF (100 mL) and cooled in a CH₃CN/dry ice bath. LDA solution (2.0 M THF/heptane/ethylbenzene, 6 mL, 12 mmol, 1.3 equiv.) was added dropwise over a period of 3 min, the reaction mixture was stirred at CH₃CN/dry ice temperature for 5 min, then warmed to ambient temperature and stirred for 4 h. H₂O (0.5 mL) was added, the mixture was stirred for 5 min, then concentrated to a dark brown oil. CH₂Cl₂ and hexane were added and removed to give a dark green/brown syrup/residue that was dissolved in CH₂Cl₂ and purified by vacuum flash chromatography on silica (15 cm h×4 cm i.d.): % CH₂Cl₂/hexane—25% (200 mL), 50% (100 mL) to give a colorless syrup that slowly solidified (3.64 g). The crude product was purified twice by radial chromatography (4 mm silica): hexane (100 mL) to afford Compound 4 (3.52 g, 93%) as a white crystalline solid: ¹H NMR (500 MHz, CDCl₃) δ 7.64 (dd, 1H, J=10.5 Hz, J=2.0 Hz), 7.41 (dd, 1H, J=8.5 Hz, J=5.5 Hz), 6.94 (td, 1H, J=9.0 Hz, J=2.0 Hz), 6.68 (t, 1H, ³J_{SnH}=9.0 Hz), 1.70 (s, 9H), 0.30 (t, 9H, ²J_{SnH}=28.0 Hz).

### tert-Butyl 2-(2-chloropyrimidin-5-yl)-6-fluoro-1H-indole-1-carboxylate (Compound 7) and 2-(2-Chloropyrimidin-5-yl)-6-fluoro-1H-indole (Compound 8)

Compound 4 (0.38 g, 9.55×10⁻⁴ mol), 5-bromo-2-chloropyrimidine (0.25 g, 1.29 mmol, 1.4 equiv.), Pd(PPh₃)₄ (80 mg, 6.92×10⁻⁵ mol, 0.07 equiv.), and toluene (25 mL) were stirred at reflux under N_{2(g)} for 15 h, then cooled to ambient temperature and stirred for 3 h. The reaction mixture was poured onto dry silica (55 mm h×45 mm i.d.) and eluted under vacuum: hexane (50 mL), CH₂Cl₂ (100 mL), % MeOH/CH₂Cl₂—1% (100 mL), 2.5% (200 mL), 5% (100 mL), 10% (50 mL) to give an orange/brown residue (0.33 g). Purification by radial chromatography (2 mm silica): hexane/EtOAc/NEt₃ v/v/v 90:8:2 (100 mL), 75:20:5 (100 mL), 50:45:5 (50 mL) afforded Compound 7 (57 mg, 17%) as an off-white solid, and Compound 8 (30 mg, 13%) as a light tan solid.

### Compound 7:

¹H NMR (300 MHz, CDCl₃) δ 8.68 (s, 2H), 7.94 (dd, 1H, J=10.5 Hz, J=2.4 Hz), 7.53 (dd, 1H, J=8.4 Hz, J=5.4 Hz), 7.06 (td, 1H, J=8.7 Hz, J=2.4 Hz), 6.68 (d, 1H, J=0.6 Hz), 1.48 (s, 9H); FIRMS (ESI) [M+H]⁺ Calcd for C₁₇H₁₆ClFN₃O₂: 348.0910, found: 348.0902.

### Compound 8:

¹H NMR (300 MHz, acetone-d₆) δ 11.10 (br s, 1H), 9.15 (s, 2H), 7.64 (dd, 1H, J=8.7 Hz, J=5.4 Hz), 7.19 (m—overlapping resonances, 2H), 6.91 (ddd, 1H, J=9.9 Hz, J=8.7 Hz, J=2.1 Hz); HRMS (ESI) [M+H]⁺ Calcd for C₁₂H₈ClFN₃: 248.0385, found: 248.0382.

### tert-Butyl 2-(2-chloropyrimidin-5-yl)-6-cyano-1H-indole-1-carboxylate (Compound 9) and 2-(2-Chloropyrimidin-5-yl)-1H-indole-6-carbonitrile (Compound 10)

1,4-Dioxane was purged with N_{2(g)} for 40 min. Compound 5 (0.46 g, 1.14 mmol), 5-bromo-2-chloropyrimidine (0.23 g, 1.19 mmol), Pd(dppf)Cl₂ (78 mg, 1.07×10⁻⁴ mol, 0.09 equiv.), and Na₂CO₃ (0.14 g, 1.32 mmol, 1.2 equiv.) were flushed with N_{2(g)} for 15 min, then 1,4-dioxane (20 mL) was added. The reaction mixture was stirred at reflux under N_{2(g)} for 4 h, cooled, and the solvent was removed to give a brown residue that was dissolved in CH₂Cl₂, poured onto dry silica (55 mm h×45 mm i.d.), and eluted under vacuum: CH₂Cl₂ (100 mL), % MeOH/CH₂Cl₂—1% (100 mL), 2% (100 mL), 3% (200 mL), 4% (100 mL) to give an orange/brown residue (0.16 g, crude Compound 9) and an orange/brown solid (62 mg, crude Compound 10).

### Compound 9:

The residue was dissolved in CH₂Cl₂/MeOH, poured onto dry silica (33 mm h×33 mm i.d.), and eluted under vacuum: % MeOH/CH₂Cl₂—1% (100 mL) 2% (100 mL) to give a dark orange residue that was dried under vacuum briefly. The residue was dissolved/suspended in CHCl₃ (1 mL), filtered, and the precipitate was rinsed with CHCl₃ (1 mL×2). The filtrate was purified by radial chromatography (1 mm silica): 75:20:5 v/v/v hexane/EtOAc/NEt₃ (100 mL) to afford Compound 9 (38 mg, 9%) as a white solid: ¹H NMR (300 MHz, CDCl₃) δ 8.71 (s, 2H), 8.58 (d, 1H, J=0.6 Hz), 7.69 (d, 1H, J=8.1 Hz), 7.56 (dd, 1H, J=8.1 Hz, J=1.5 Hz), 6.77 (s, 1H), 1.49 (s, 9H); HRMS (ESI) [M+H]⁺ Calcd for C₁₈H₁₆O₂N₄Cl: 355.0956, found: 355.0958.

### Compound 10:

The solid was dissolved/suspended in CHCl₃ (2.5 mL), filtered, and the precipitate was rinsed with CHCl₃ (2.5 mL×3) then dried under vacuum to afford Compound 10 (29 mg, 10%) as a yellow/orange solid: ¹H NMR (400 MHz, DMSO-d₆) δ 12.44 (s, 1H), 9.31 (s, 2H), 7.96 (s, 1H), 7.79 (d, 1H, J=8.0 Hz), 7.40 (d, 1H, J=8.0 Hz), 7.36 (s, 1H).

### tert-Butyl 2-(2-chloropyrimidin-5-yl)-1H-indole-1-carboxylate (Compound 11)

1-Boc-indole-2-boronic acid (Compound 6) (0.25 g, 9.58×10⁻⁴ mol), 5-bromo-2-chloropyrimidine (0.18 g, 9.31×10⁻⁴ mol), and Pd(dppf)Cl₂ (52 mg, 7.11×10⁻⁵ mol, 0.08 equiv.) were flushed with N_{2(g)} for 10 min, then 1,4-dioxane (10 mL) was added followed by a solution of K₂CO₃ in H₂O (1.5 mL, 2 M, 3 mmol, 3.2 equiv.). The reaction mixture was stirred at reflux under N_{2(g)} for 7 h, cooled to ambient temperature, stirred overnight, then filtered through Celite, and the Celite was rinsed with 1,4-dioxane. The filtrate was concentrated to brown oil, then CHCl₃ and hexane were added and removed to give a brown syrup that was dissolved in CH₂Cl₂, poured onto dry silica (45 mm h×45 mm i.d.) and eluted under vacuum: CH₂Cl₂ (200 mL), % MeOH/CH₂Cl₂—1% (100 mL), 2% (50 mL), 3% (50 mL) to give a tan solid (0.19 g). Purification by radial chromatography (2 mm silica): CH₂Cl₂ (100 mL) gave an off-white solid (0.11 g) that was again purified by radial chromatography (2 mm silica): hexane/EtOAc/NEt₃ v/v/v 90:8:2 (35 mL), 85:12:3 (100 mL), 75:20:5 (50 mL) to afford Compound 11 (82 mg, 27%) as a white solid: ¹H NMR (400 MHz, CDCl₃) δ 8.69 (s, 2H), 8.20 (d, 1H, J=8.4 Hz), 7.60 (d, 1H, J=7.6 Hz), 7.40 (ddd, 1H, J=8.4 Hz, J=7.2 Hz, J=1.2 Hz), 7.30 (ddd, 1H, J=7.8 Hz, J=7.2 Hz, J=0.8 Hz), 6.71 (s, 1H), 1.49 (s, 9H).

### tert-Butyl 6-fluoro-2-(2-(piperidin-1-yl)pyrimidin-5-yl)-1H-indole-1-carboxylate (Compound 12)

Compound 7 (42 mg, 1.21×10⁻⁴ mol), piperidine (0.05 mL, 5.1×10⁻⁴ mol, 4.2 equiv.), K₂CO₃ (24 mg, 1.74×10⁻⁴ mol, 1.4 equiv.), and CH₃CN (10 mL) were stirred at reflux under N_{2(g)} for 75 min, then cooled to ambient temperature. The CH₃CN was removed to give a residue that was dissolved in CH₂Cl₂, poured onto dry silica (33 mm h×33 mm i.d.), and eluted under vacuum: CH₂Cl₂ (25 mL), % MeOH/CH₂Cl₂—1% (50 mL), 2% (75 mL), 3% (50 mL) to afford Compound 12 (47 mg, 98%) as a light tan solid: ¹H NMR (500 MHz, CDCl₃) δ 8.33 (s, 2H), 7.93 (dd, 1H, J=10.5 Hz, J=2.5 Hz), 7.45 (dd, 1H, J=8.5 Hz, J=5.5 Hz), 7.01 (td, 1H, J=8.5 Hz, J=2.5 Hz), 6.48 (s, 1H), 3.84 (t, 4H, J=5.0 Hz), 1.71 (m, 2H), 1.62 (m, 4H), 1.48 (s, 9H).

### tert-Butyl 6-fluoro-2-(2-(4-fluoropiperidin-1-yl)pyrimidin-5-yl)-1H-indole-1-carboxylate (Compound 13)

Compound 7 (60 mg, 1.73×10⁻⁴ mol), 4-fluoropiperidine·HCl (34 mg, 2.44×10⁻⁴ mol, 1.4 equiv.), K₂CO₃ (88 mg, 6.37×10⁻⁴ mol, 3.7 equiv.), and CH₃CN (5 mL) were stirred at reflux under N_{2(g)} for 5 h, then cooled. The solvent was removed to give a residue that was dissolved in CH₂Cl₂, poured onto dry silica (33 mm h×33 mm i.d.), and eluted under vacuum with CH₂Cl₂ (125 mL) to afford Compound 13 (64 mg, 89%) as a white foam: ¹H NMR (300 MHz, CDCl₃) δ 8.35 (s, 2H), 7.93 (dd, 1H, J=10.8 Hz, J=2.4 Hz), 7.46 (dd, 1H, J=8.7 Hz, J=5.7 Hz), 7.01 (td, 1H, J=8.7 Hz, J=2.4 Hz), 6.50 (s, 1H), 4.99 (tt, 0.5H, J=6.3 Hz, J=3.3 Hz) and 4.83 (dt, 0.5H, J=9.3 Hz, J=4.8 Hz) (²J_{HF}=48.3 Hz), 3.97 (t, 4H, J=5.7 Hz), 1.93 (m, 4H), 1.48 (s, 9H); HRMS (ESI) [M+H]⁺ Calcd for C₂₂H₂₅F₂N₄O₂: 415.1940, found: 415.1944.

### tert-Butyl 2-(2-(4,4-difluoropiperidin-1-yl)pyrimidin-5-yl)-6-fluoro-1H-indole-1-carboxylate (Compound 14)

Compound 7 (94.5 mg, 2.72×10⁻⁴ mol), 4,4-difluoropiperidine HCl (60 mg, 3.81×10⁻⁴ mol, 1.4 equiv.), K₂CO₃ (186 mg, 1.35 mmol, 5 equiv.), and CH₃CN (20 mL) were stirred at reflux under N_{2(g)} for 21 h, then cooled to ambient temperature, filtered, and the precipitate was rinsed with CH₃CN. The CH₃CN was removed to give an orange residue, then CH₂Cl₂ and hexane were added and removed to give a light orange solid that was dried under vacuum. The solid was dissolved in CH₂Cl₂, poured onto dry silica (45 mm h×45 mm i.d.), and eluted under vacuum with CH₂Cl₂ (350 mL) to give a colorless residue. Purification by radial chromatography (2 mm silica): % CH₂Cl₂/hexane—25% (50 mL), 50% (125 mL) gave a white solid (46 mg) that was purified again by radial chromatography (1 mm silica): % CH₂Cl₂/hexane—25% (100 mL), 35% (100 mL), 50% (50 mL) to afford Compound 14 (32 mg, 27%) as a white solid: ¹H NMR (400 MHz, CDCl₃) δ 8.37 (s, 2H), 7.93 (dd, 1H, J=10.8 Hz, J=2.0 Hz), 7.46 (dd, 1H, J=8.4 Hz, J=5.6 Hz), 7.02 (td, 1H, J=8.8 Hz, J=2.0 Hz), 6.51 (s, 1H), 4.03 (t, 4H, J=6.0 Hz), 2.02 (septet, 4H, J=6.0 Hz), 1.48 (s, 9H) HRMS (ESI) [M+H]⁺ Calcd for C₂₂H₂₄O₂N₄F₃: 433.1846, found: 433.1836.

### 6-Fluoro-2-(2-(piperidin-1-yl)pyrimidin-5-yl)-1H-indole (Compound 15)

Compound 12 (41 mg, 1.03×10⁻⁴ mol) was dissolved in TFA (1 mL, 13 mmol, 126 equiv.), stirred at ambient temperature for 15 min, then poured into a mixture of NaHCO₃ (1.23 g, 14.64 mmol, 1.1 equiv. TFA), H₂O (30 mL), and CH₂Cl₂ (30 mL). The mixture was stirred, the layers were separated, and the aqueous layer was extracted with CH₂Cl₂ (10 mL). The combined CH₂Cl₂ layers were washed with brine (15 mL) and dried over MgSO₄. The solution was concentrated, poured onto dry silica (33 mm h×33 mm i.d.), and eluted under vacuum: hexane/EtOAc/NEt₃ v/v/v 90:8:2 (50 mL), 75:20:5 (50 mL), 50:45:5 (75 mL) to afford Compound 15 (27 mg, 88%) as a light-yellow solid: ¹H NMR (300 MHz, CDCl₃) δ 8.56 (s, 2H), 8.20 (br s, 1H), 7.49 (dd, 1H, J=8.7 Hz, J=5.4 Hz), 7.08 (dd, 1H, J=9.6 Hz, J=2.1 Hz), 6.88 (ddd, 1H, J=9.6 Hz, J=8.7 Hz, J=2.4 Hz), 6.62 (dd, 1H, J=2.1 Hz, J=0.9 Hz), 3.84 (t, 4H, J=5.4 Hz), 1.68 (m, 6H); ¹³C NMR (125 MHz, DMSO-d₆) δ 160.08, 158.71 (d, ¹J_{FC}=234.4 Hz), 154.49, 136.68 (d, J_{FC}=13.0 Hz), 134.17 (d, J_{FC}=3.4 Hz), 125.44, 120.46 (d, J_{FC}=10.2 Hz), 114.48, 107.73 (d, J_{FC}=24.1 Hz), 97.05 (d, J_{FC}=25.7 Hz), 96.91, 44.34, 25.25, 24.27; ¹⁹F NMR (470.6 MHz, DMSO-d₆) δ −121.61 (m); HRMS (ESI) [M+H]⁺ Calcd for C₁₇H₁₈FN₄: 297.1510, found: 297.1515.

### 6-Fluoro-2-(2-(4-fluoropiperidin-1-yl)pyrimidin-5-yl)-1H-indole (Compound 16)

Compound 13 (59 mg, 1.42×10⁻⁴ mol) was dissolved in TFA (1.5 mL, 19.5 mmol, 137 equiv.), stirred at ambient temperature for 20 min, then poured into a mixture of NaHCO₃ (1.82 g, 21.67 mmol, 1.1 equiv. TFA), H₂O (45 mL), and CH₂Cl₂ (45 mL). The mixture was stirred until the color was gone, then the layers were separated, and the aqueous layer was extracted with CH₂Cl₂ (10 mL). The combined CH₂Cl₂ layers were washed with brine (20 mL) and dried over MgSO₄. The solution was concentrated, poured onto dry silica (33 mm h×33 mm i.d.), and eluted under vacuum: hexane/EtOAc/NEt₃ v/v/v 90:8:2 (50 mL), 75:20:5 (50 mL), 50:45:5 (100 mL) to afford Compound 16 (44 mg, 99%) as a light tan solid: ¹H NMR (300 MHz, CDCl₃) δ 8.58 (s, 2H), 8.21 (br s, 1H), 7.50 (dd, 1H, J=8.7 Hz, J=5.4 Hz), 7.08 (dd, 1H, J=9.6 Hz, J=2.1 Hz), 6.89 (ddd, 1H, J=9.6 Hz, J=8.7 Hz, J=2.1 Hz), 6.64 (dd, 1H, J=2.1 Hz, J=0.9 Hz), 5.00 (tt, 0.5H, J=6.0 Hz, J=3.3 Hz) and 4.83 (dt, 0.5H, J=9.6 Hz, J=4.8 Hz) (²J_{HF}=48.3 Hz), 3.97 (m, 4H), 1.96 (m, 4H); ¹H NMR (300 MHz, DMSO-d₆) δ 11.58 (s, 1H), 8.83 (s, 2H), 7.49 (dd, 1H, J=8.7 Hz, J=5.4 Hz), 7.13 (dd, 1H, J=9.9 Hz, J=2.1 Hz), 6.85 (ddd, 1H, J=9.9 Hz, J=8.7 Hz, J=2.1 Hz), 6.82 (d, 1H, J=1.8 Hz), 4.93 (dtt, 1H, ²J_{HF}=48.6 Hz, J=7.2 Hz, J=3.6 Hz), 3.97 (m, 2H), 3.78 (m, 2H), 1.93 (m, 2H), 1.72 (m, 2H); HRMS (ESI) [M+H]⁺ Calcd for C₁₇H₁₇F₂N₄: 315.1416, found: 315.1420.

### 2-(2-(4,4-Difluoropiperidin-1-yl)pyrimidin-5-yl)-6-fluoro-1H-indole (Compound 17)

Compound 14 (27 mg, 6.24×10⁻⁵ mol) was dissolved in TFA (0.6 mL, 7.8 mmol, 125 equiv.), stirred at ambient temperature for 20 min, then poured into a mixture of NaHCO₃ (0.856 g, 10.19 mmol, 1.3 equiv. TFA), H₂O (20 mL), and CH₂Cl₂ (20 mL). The mixture was stirred for 15 min, then the layers were separated, and the aqueous layer was extracted with CH₂Cl₂ (5 mL×2). The combined CH₂Cl₂ layers were washed with brine (15 mL) and dried over MgSO₄. The solution was poured onto dry silica (33 mm h×33 mm i.d.), and eluted under vacuum: CH₂Cl₂ (50 mL) % MeOH/CH₂Cl₂—1% (50 mL), 2% (25 mL), 4% (50 mL) to afford Compound 17 (18 mg, 87%) as an off-white solid: ¹H NMR (300 MHz, CDCl₃) δ 8.60 (s, 2H), 8.23 (br s, 1H), 7.51 (dd, 1H, J=8.7 Hz, J=5.4 Hz), 7.08 (dd, 1H, J=9.6 Hz, J=2.1 Hz), 6.90 (ddd, 1H, J=9.6 Hz, J=8.7 Hz, J=2.1 Hz), 6.66 (d, 1H, J=1.5 Hz), 4.03 (t, 4H, J=5.7 Hz), 2.04 (tt, 4H, ³J_{HF}=13.5 Hz, J=5.7 Hz); HRMS (ESI) [M+H]⁺ Calcd for C₁₇H₁₆F₃N₄: 333.1322, found: 333.1314.

### 2-(2-(3-Fluoro-4-hydroxypyrrolidin-1-yl)pyrimidin-5-yl)-1H-indole-6-carbonitrile (Compound 18)

Compound 10 (29 mg, 1.14×10⁻⁴ mol), trans/racemic-4-fluoro-3-hydroxypyrrolidine·HCl (33 mg, 2.33×10⁻⁴ mol, 2 equiv.), K₂CO₃ (120 mg, 8.68×10⁻⁴ mol, 7.6 equiv.), and CH₃CN (10 mL) were stirred at reflux under N_{2(g)} for 3 h, cooled, and CH₃CN was removed to give a dark-orange residue. The residue was dissolved/suspended in CH₂Cl₂/MeOH, filtered, and the precipitate was rinsed with CH₂Cl₂. The solvent was removed from the filtrate to give an orange residue that was dried under vacuum briefly (56 mg). The residue was dissolved in CH₂Cl₂/MeOH, poured onto dry silica (33 mm h×33 mm i.d.), and eluted under vacuum: % MeOH/CH₂Cl₂—1% (100 mL), 2.5% (100 mL), 5% (100 mL) to afford Compound 18 (9 mg, 24%) as a light-tan solid: ¹H NMR (300 MHz, DMSO-d₆) δ 12.08 (s, 1H), 8.92 (s, 2H), 7.82 (s, 1H), 7.67 (d, 1H, J=8.1 Hz), 7.33 (dd, 1H, J=8.1 Hz, J=1.5 Hz), 6.98 (d, 1H, J=1.5 Hz), 5.61 (d, 1H, J=3.9 Hz), 5.10 (dm, 1H, ²J_{HF}=49.8 Hz), 4.37 (m, 1H), 3.88 (s, 1H), 3.77 (m, 1H), 3.69 (s, 2H), HRMS (ESI) [M−H]⁺ Calcd for C₁₇H₁₃FN₅O: 322.1099, found: 322.1093.

### tert-Butyl 6-fluoro-2-(2-(3-fluoro-4-hydroxypyrrolidin-1-yl)pyrimidin-5-yl)-1H-indole-1-carboxylate (Compound 19)

Compound 7 (56 mg, 1.61×10⁻⁴ mol), trans/racemic-4-fluoro-3-hydroxypyrrolidine·HCl (52 mg, 3.67×10⁻⁴ mol, 2.3 equiv.), K₂CO₃ (168 mg, 1.22 mmol, 7.5 equiv.), and CH₃CN (15 mL) were stirred at reflux under N_{2(g)} for 3 h, then cooled to ambient temperature. The CH₃CN was removed to give an off-white solid that was dissolved/suspended in CH₂Cl₂, filtered, and the precipitate was rinsed with CH₂Cl₂. The CH₂Cl₂ was removed from the filtrate to give a yellow syrup that was dried under vacuum (89 mg), then dissolved in CH₂Cl₂/MeOH, poured onto dry silica (45 mm h×45 mm i.d.), and eluted under vacuum: % MeOH/CH₂Cl₂—1% (50 mL), 2% (75 mL), 3% (100 mL), 4% (50 mL). The desired fractions were combined and purified by radial chromatography (1 mm silica): % MeOH/CH₂Cl₂—1% (50 mL), 2% (75 mL), 3% (25 mL) to afford Compound 19 (41 mg, 61%) as an off-white solid: ¹H NMR (300 MHz, CDCl₃) δ 8.39 (s, 2H), 7.91 (dd, 1H, J=10.8 Hz, J=2.4 Hz), 7.46 (dd, 1H, J=8.7 Hz, J=5.7 Hz), 7.01 (td, 1H, J=8.7 Hz, J=2.4 Hz), 5.11 (dm, 1H, ²J_{HF}=51.0 Hz), 4.59 (dm, 1H, J=6.9 Hz), 4.03 (m, 1H), 3.90 (m, 3H), 1.92 (br d, 1H, J=2.7 Hz), 1.50 (s, 9H).

### tert-Butyl 2-(2-(3-fluoro-4-hydroxypyrrolidin-1-yl)pyrimidin-5-yl)-1H-indole-1-carboxylate (Compound 20)

Compound 11 (79 mg, 2.40×10-mol), trans/racemic-4-fluoro-3-hydroxypyrrolidine·HCl (97 mg, 6.85×10⁻⁴ mol, 2.9 equiv.), K₂CO₃ (158 mg, 1.14 mmol, 4.7 equiv.), and CH₃CN (15 mL) were stirred at reflux under N_{2(g)} for 5 h, then cooled to ambient temperature. The CH₃CN was removed to give a residue that was dried under vacuum briefly, then dissolved in CH₂Cl₂, poured onto dry silica (33 mm h×33 mm i.d.), and eluted under vacuum: CH₂Cl₂ (25 mL), % MeOH/CH₂Cl₂—1% (50 mL), 2% (75 mL), 3% (50 mL) to afford Compound 20 (85 mg, 89%) as an off-white foam: ¹H NMR (300 MHz, CDCl₃) δ 8.41 (s, 2H), 8.17 (d, 1H, J=8.4 Hz), 7.55 (d, 1H, J=7.5 Hz), 7.33 (partially resolved ddd, 1H, J=7.8 Hz, J=1.2 Hz), 7.25 (m—partially obscured by CHCl₃ resonance, 1H), 6.55 (s, 1H), 5.10 (dm, 1H, ²J_{HF}=50.7 Hz), 4.59 (m, 1H), 4.03 (m, 1H), 3.91 (m, 3H), 1.89 (d, 1H, J=3.9 Hz), 1.50 (s, 9H).

### 4-Fluoro-1-(5-(6-fluoro-1H-indol-2-yl)pyrimidin-2-yl)pyrrolidin-3-ol (Compound 21)

Compound 19 (27 mg, 6.48×10⁻⁵ mol) was dissolved in TFA (0.75 mL, 9.7 mmol, 150 equiv.), stirred at ambient temperature for 15 min, then poured into a mixture of NaHCO₃ (0.91 g, 10.83 mmol, 1.1 equiv. TFA), H₂O (25 mL), and CH₂Cl₂ (25 mL). The mixture was stirred for 5 min, then the layers were separated, and the aqueous layer was extracted with CH₂Cl₂ (5 mL×2). The combined CH₂Cl₂ layers were dried over MgSO₄ and the solvent was removed to give an off-white solid that was dried under vacuum to afford Compound 21(17 mg, 83%): ¹H NMR (300 MHz, acetone-d₆) δ 10.71 (br s, 1H), 8.80 (s, 2H), 7.52 (dd, 1H, J=8.7 Hz, J=5.4 Hz), 7.11 (dd, 1H, J=9.9 Hz, J=2.1 Hz), 6.84 (ddd, 1H, J=9.9 Hz, J=8.7 Hz, J=2.4 Hz), 6.79 (m, 1H), 5.12 (dm, 1H, ²J_{HF}=51.0 Hz), 4.67 (d, 1H, J=3.9 Hz), 4.51 (m, 1H), 3.96 (d, 1H, J=2.1 Hz), 3.85 (m, 1H), 3.79 (m, 2H); ¹³C NMR (125 MHz, DMSO-d₆) δ 158.74 (d, ¹J_{FC}=234.6 Hz), 159.10, 154,57, 136.75 (d, J_{FC}=12.6 Hz), 134.13 (d, J_{FC}=3.5 Hz), 125.43, 120.51, 115.14, 107.76 (d, J_{FC}=24.3 Hz), 97.09 (d, J_{FC}=25.5 Hz), 97.07, 95.16 (d, J_{FC}=177.0 Hz), 71.40 (d, J_{FC}=27.2 Hz), 52.55, 50.71 (d, J_{FC}=21.9 Hz); ¹⁹F NMR (470.6 MHz, DMSO-d₆) δ −121.54 (m), -181.85 (m); HRMS (ESI) [M−H]⁺ Calcd for C₁₆H₁₃F₂N₄O: 315.1052, found: 315.1048.

### 1-(5-(1H-Indol-2-yl)pyrimidin-2-yl)-4-fluoropyrrolidin-3-ol (Compound 22)

Compound 20 (79 mg, 1.98×10⁻⁴ mol) was dissolved in CH₂Cl₂ (5 mL), HCl/1,4-dioxane (4 M, 1 mL, 4 mmol) was added, the mixture was stirred for 5 h, then filtered, and the precipitate was rinsed with EtOEt (2 mL×3). The solvent was removed from the filtrate to give a light-yellow solid that was dried under vacuum briefly, then dissolved in CH₂Cl₂ (10 mL). H₂O (10 mL) was added followed by conc. NH₄OH_{(aq)} (5 drops) and the mixture was stirred for 5 min (pH 11-12). Brine (5 mL) was added, the layers were mixed and separated, and the CH₂Cl₂ layer was dried over MgSO₄. Analysis by TLC (3% MeOH/CH₂Cl₂) indicated N-Boc protected Compound 20 remained. The solvent was removed to give a white foam that was dried under vacuum (50 mg). The foam was dissolved in TFA (2 mL, 26 mmol), stirred for 15 min, then poured into a mixture of CH₂Cl₂ (40 mL), H₂O (40 mL), and NaHCO₃ (2.42 g, 28.81 mmol, 1.1 equiv. TFA). The mixture was stirred for 15 min, then the layers were separated, and the H₂O layer was extracted with CH₂Cl₂ (10 mL). The combined CH₂Cl₂ layers were washed with brine (25 mL), dried over MgSO₄, and the solvent was removed to give an off-white solid (24 mg). Purification by flash column chromatography on silica (3% MeOH/CH₂Cl₂) afforded Compound 22 (21 mg, 36%) as an off-white solid: ¹H NMR (400 MHz, DMSO-d₆) δ 11.47 (s, 1H), 8.86 (s, 2H), 7.50 (d, 1H, J=8.0 Hz), 7.38 (d, 1H, J=8.0 Hz), 7.08 (partially resolved ddd, 1H, J=7.2 Hz, J=1.2 Hz), 6.99 (partially resolved ddd, 1H, J=7.2 Hz, J=1.2 Hz), 6.81 (d 1H, J=1.2 Hz), 5.60 (d, 1H, J=3.6 Hz), 5.09 (dm, 1H, ²J_{HF}=50.8 Hz), 4.37 (m, 1H), 3.86 (s, 1H), 3.79 (m, 1H), 3.67 (s, 2H); HRMS (ESI) [M−H]⁺ Calcd for C₁₆H₁₄FN₄O: 297.1152, found: 297.1145.

### 5-Bromo-2-(4-methoxypiperidin-1-yl)pyrimidine (Compound 23)

5-Bromo-2-chloropyrimidine (0.88 g, 4.55 mmol), 4-methoxypiperidine (0.65 mL, 5.25 mmol, 1.2 equiv.), K₂CO₃ (0.89 g, 6.44 mmol, 1.4 equiv.), and CH₃CN (25 mL) were stirred at reflux under N_{2(g)} for 1 h, then cooled to ambient temperature, filtered, and the precipitate was rinsed with CH₃CN. The CH₃CN was removed to give a faint-yellow residue that was dissolved in CH₂Cl₂, poured onto dry silica (55 mm h×45 mm id.), and eluted under vacuum: CH₂Cl₂ (50 mL), % MeOH/CH₂Cl₂—1% (100 mL), 2% (100 mL), 3% (100 mL), 4% (100 mL) to afford Compound 23 (1.21 g, 98%) as a white solid: ¹H NMR (300 MHz, CDCl₃) δ 8.27 (s, 2H), 4.19 (m, 2H), 3.44 (m, 3H), 3.39 (s, 3H), 1.92 (m, 2H), 1.57 (m, 2H).

### 4-(5-Bromopyrimidin-2-yl)morpholine (Compound 24)

5-Bromo-2-chloropyrimidine (1.48 g, 7.65 mmol), morpholine (1 mL, 11.6 mmol, 1.5 equiv.), K₂CO₃ (1.30 g, 9.41 mmol, 1.2 equiv.), and CH₃CN (55 mL) were stirred at reflux under N_{2(g)} for 3 h, then cooled to ambient temperature, filtered, and the precipitate was rinsed with CH₃CN. The solvent was removed from the filtrate to give a white solid that was dissolved in CH₂Cl₂, poured onto dry silica (55 mm h×45 mm i.d.), and eluted under vacuum: CH₂Cl₂ (100 mL), % MeOH/CH₂Cl₂—1% (100 mL), 2% (100 mL), 3% (100 mL), 4% (100 mL) to afford Compound 24 (1.74 g, 93%) as a white solid: ¹H NMR (300 MHz, CDCl₃) δ 8.31 (s, 2H), 3.76 (s, 8H); ¹³C NMR (125 MHz, CDCl₃) δ 160.15, 158.09, 106.39, 66.85, 44.52; HRMS (ESI) [M+H]⁺ Calcd for C₈H₁₁BrN₃O: 244.0080, found: 244.0084.

### (R)-5-Bromo-2-(3-fluoropyrrolidin-1-yl)pyrimidine ((R)-Compound 25)

5-Bromo-2-chloropyrimidine (0.51 g, 2.64 mmol), (R)-3-fluoropyrrolidine·HCl (0.49 g, 3.90 mmol, 1.5 equiv.), K₂CO₃ (1.13 g, 8.18 mmol, 3.1 eqiuv.), and CH₃CN (30 mL) were stirred at reflux under N_{2(g)} for 90 min, then cooled to ambient temperature, filtered, and the precipitate was rinsed with EtOAc 3×. The solvent was removed from the filtrate to give a white solid that was dissolved in CH₂Cl₂, poured onto dry silica (55 mm h×45 mm i.d.), and eluted under vacuum: % CH₂Cl₂/hexane—50% (50 mL), 75% (100 mL), CH₂Cl₂ (200 mL), % MeOH/CH₂Cl₂—1% (100 mL), 2% (100 mL), 3% (100 mL), 5% (100 mL), 10% (50 mL) to afford (R)-Compound 25 (0.50 g, 77%) as an off-white solid: ¹H NMR (300 MHz, CDCl₃) δ 8.32 (s, 2H), 5.36 (dt, 1H, ²J_{HF}=52.8, J=3.3 Hz), 3.99-3.57 (m, 4H), 2.45-1.99 (m, 2H).

### 1-(5-Bromopyrimidin-2-yl)piperidin-4-ol (Compound 26)

5-Bromo-2-chloropyrimidine (0.97 g, 5.01 mmol), 4-hydroxy-piperidine (0.56 g, 5.54 mmol, 1.1 equiv.), K₂CO₃ (0.92 g, 6.66 mmol, 1.3 equiv.), and CH₃CN (30 mL) were stirred at reflux under N_{2(g)} for 1 h, then cooled to ambient temperature, filtered, and the precipitate was rinsed with CH₃CN. The CH₃CN was removed from the filtrate to give a solid that was dissolved/suspended in CH₂Cl₂, poured onto dry silica (55 mm h×45 mm i.d.), and eluted under vacuum: % MeOH/CH₂Cl₂—1% (100 mL), 2% (100 mL), 3% (200 mL), 5% (150 mL) to afford Compound 26 (1.27 g, 98%) as a white solid: ¹H NMR (300 MHz, CDCl₃) δ 8.27 (s, 2H), 4.33 (dtd, 2H, J=13.8 Hz, J=4.8 Hz, J=0.9 Hz), 3.96 (apparent octet, 1H, J=4.2 Hz), 3.33 (ddd, 2H, J=13.5 Hz, J=9.6 Hz, J=3.3 Hz), 1.94 (m, 2H), 1.53 (m, 3H).

### 1-(5-Bromopyrimidin-2-yl)piperidin-4-one (Compound 27)

Compound 26 (0.54 g, 2.09 mmol) was dissolved in CH₂Cl₂ (25 mL), then H₂O (0.05 mL) was added, followed by Dess-Martin periodinane (1.03 g, 2.43 mmol, 1.2 equiv.). The reaction mixture was stirred at reflux for 7 h, then cooled to ambient temperature, stirred for 17 h, and poured into a mixture of CH₂Cl₂ (25 mL), H₂O (50 mL), Na₂S₂O₃·5 H₂O (2.17 g, 8.74 mmol), and NaHCO₃ (2.25 g, 26.78 mmol). The mixture was stirred for 2 h, then the layers were separated, and the H₂O layer was extracted with CH₂Cl₂ (10 mL×2). The combined CH₂Cl₂ layers were washed with brine (25 mL) and dried over MgSO₄. The solution was concentrated, poured onto dry silica (55 mm h×45 mm i.d.), and eluted under vacuum: CH₂Cl₂ (200 mL), % MeOH/CH₂Cl₂—1% (100 mL), 2.5% (100 mL), 5% (200 mL) to give an off-white solid (0.55 g). Purification by radial chromatography (4 mm silica): % MeOH/CH₂Cl₂—1% (100 mL), 2% (100 mL), 4% (50 mL), 5% (100 mL) afforded recovered Compound 26 (0.11 g, 20%) and Compound 27 (0.38 g, 71%) as a white solid: ¹H NMR (300 MHz, CDCl₃) δ 8.35 (s, 2H), 4.10 (t, 4H, J=6.3 Hz), 2.50 (t, 4H, J=6.3 Hz).

### 5-Bromo-2-(piperidin-1-yl)pyridine (Compound 28)

2,5-Dibromopyridine (0.87 g, 3.67 mmol) and piperidine (5 mL, 50.6 mmol, 13.8 equiv.) were stirred under N_{2(g)} in a heated sand bath (105° C.) for 15 h, then cooled to ambient temperature. EtOAc (5 mL) was added, the mixture was filtered, and the precipitate was rinsed with EtOAc (5 mL×2). The filtrate was concentrated to an oil/residue, then dissolved in CH₂Cl₂, poured onto dry silica (55 mm h×45 mm i.d.), and eluted under vacuum: hexane (50 mL), hexane/EtOAc/NEt₃ v/v/v 90:8:2 (100 mL), 75:20:5 (100 mL) to afford Compound 28 (0.86 g, 97%) as a colorless oil: ¹H NMR (300 MHz, CDCl₃) δ 8.16 (d, 1H, J=2.4 Hz), 7.48 (dd, 1H, J=9.3 Hz, J=2.4 Hz), 6.54 (d, 1H, J=9.3 Hz), 3.50 (m, 4H), 1.63 (m, 6H).

### 4-(5-Bromopyridin-2-yl)morpholine (Compound 29)

2,5-Dibromopyridine (0.87 g, 3.67 mmol) and morpholine (5 mL, 58 mmol, 16 equiv.) were stirred under N_{2(g)} in a heated sand bath (−110° C.) for 15 h, then cooled to ambient temperature. EtOAc (5 mL) was added, the mixture was filtered, and the precipitate was rinsed with EtOAc (5 mL×2). The filtrate was concentrated, diluted with hexane, poured onto dry silica (55 mm h×45 mm i.d.), and eluted under vacuum: hexane/EtOAc/NEt₃ v/v/v 90:8:2 (100 mL), 75:20:5 (200 mL) to afford Compound 29 (0.86 g, 96%) as an off-white solid: ¹H NMR (300 MHz, CDCl₃) δ 8.21 (d, 1H, J=2.4 Hz), 7.56 (dd, 1H, J=9.0 Hz, J=2.4 Hz), 6.53 (d, 1H, J=9.0 Hz), 3.81 (t, 4H, J=4.8 Hz), 3.47 (t, 4H, J=4.8 Hz).

### 4-(5-bromopyrazin-2-yl)morpholine (Compound 30)

Morpholine (0.3 mL, 3.5 mmol) was added to a suspension of NaH (87 mg, 90%, 3.26 mmol) in THE (6 mL) at 0° C. under N_{2(g)}, the mixture was stirred for 15 min, then 2,5-dibromopyrazine (0.81 g, 3.41 mmol) in THE (6 mL) was added, and the reaction mixture was stirred at reflux overnight. The solvent was evaporated, the residue was dissolved in EtOAc (20 mL), washed with H₂O (10 mL), and dried over MgSO₄. The solvent was removed and the residue was purified by flash column chromatography (4:1 v/v hexanes/EtOAc) to afford Compound 30 as a white solid (0.86 g, quantitative): ¹H NMR (CDCl₃, 500 MHz): δ 8.16 (d, 1H, J=2.0 Hz), 7.86 (d, 1H, J=2.5 Hz), 3.83 (apparent t, 4H, J=8.0 Hz), 3.53 (apparent t, 4H, J=8.0 Hz). HRMS (ESI) [M+H]⁺ Calcd for C₈H₁₁ON₃Br: 244.0080, found: 244.0076.

### tert-Butyl 6-fluoro-2-(2-(4-methoxypiperidin-1-yl)pyrimidin-5-yl)-1H-indole-1-carboxylate (Compound 31)

Compound 4 (0.65 g, 1.63 mmol), compound 23 (0.49 g, 1.80 mmol, 1.1 equiv.), Pd(dppf)Cl₂ (0.11 g, 1.50×10⁻⁴ mol, 0.09 equiv.), and Na₂CO₃ (0.56 g, 5.28 mmol, 3.2 equiv.) were flushed with N_{2(g)} for 10 min, then 1,4-dioxane (45 mL) was added. The reaction mixture was stirred at reflux under N_{2(g)} for 15 h, then cooled to ambient temperature, filtered through Celite, and the Celite was rinsed with 1,4-dioxane. The filtrate was concentrated to a brown syrup that was dried under vacuum, then dissolved in CH₂Cl₂, poured onto dry silica (55 mm h×45 mm i.d.), and eluted under vacuum: CH₂Cl₂ (100 mL), % MeOH/CH₂Cl₂—1% (100 mL), 2.5% (100 mL), 5% (150 mL) to give a brown residue (0.65 g). The residue was dissolved in CH₂Cl₂, poured onto dry silica (55 mm h×45 mm i.d.), and eluted under vacuum: hexane (25 mL), hexane/EtOAc/NEt₃ v/v/v 75:20:5 (100 mL), 50:45:5 (100 mL), 20:75:5 (150 mL) to give a dark orange solid (0.52 g). Purification by radial chromatography (2 mm silica): CH₂Cl₂ (100 mL), 1% MeOH/CH₂Cl₂ (50 mL) gave crude Compound 31 (0.30 g, light brown syrup) and crude Compound 36 (91 mg, light brown residue—further purified below).

Crude Compound 31 was purified by radial chromatography (2 mm silica): hexane/EtOAc/NEt₃ v/v/v 95:4:1 (100 mL), 90:8:2 (25 mL) to give a sticky, faint yellow residue that was again purified by radial chromatography (2 mm silica): CHCl₃ (50 mL), % EtOH/CHCl₃—1% (50 mL), 2% (25 mL) to afford Compound 31 (0.26 g, 37%) as a faint yellow viscous syrup that slowly solidified: ¹H NMR (300 MHz, CDCl₃) δ 8.34 (s, 2H), 7.93 (dd, 1H, J=10.8 Hz, J=2.4 Hz), 7.45 (dd, 1H, J=8.7 Hz, J=5.4 Hz), 7.01 (td, 1H, J=8.7 Hz, J=2.4 Hz), 6.49 (s, 1H), 4.34 (m, 2H), 3.48 (m, 3H), 3.41 (s, 3H), 1.96 (m, 2H), 1.59 (m, 2H), 1.47 (s, 9H); HRMS (ESI) [M+H]⁺ Calcd for C₂₃H₂₈FN₄O₃: 427.2140, found: 427.2131.

### tert-Butyl (R)-6-fluoro-2-(2-(3-fluoropyrrolidin-1-yl)pyrimidin-5-yl)-1H-indole-1-carboxylate ((R)-Compound 32)

Compound 4 (0.40 g, 1.00 mmol), (R)-Compound 25 (0.30 g, 1.22 mmol, 1.2 equiv.) Pd(dppf)Cl₂ (76 mg, 1.04×10⁻⁴ mol, 0.1 equiv.), Na₂CO₃ (0.17 g, 1.60 mmol, 1.6 equiv.), and 1,4-dioxane (25 mL) were stirred at reflux under N_{2(g)} for 6 h, then cooled to ambient temperature. The reaction mixture was filtered through Celite, the Celite was rinsed with EtOAC, the filtrate was concentrated to a brown syrup, then CH₂Cl₂ and hexane were added and removed to give a brown residue that was dried under vacuum. The residue was dissolved in CH₂Cl₂ (+a few drops MeOH), poured onto dry silica (55 mm h×45 mm i.d.), and eluted under vacuum: CH₂Cl₂ (100 mL), % MeOH/CH₂Cl₂—1% (100 mL), 2.5% (100 mL), 5% (200 mL) to give a dark orange/brown residue (0.37 g). The residue was dissolved in CH₂Cl₂/MeOH and purified by radial chromatography (2 mm silica): hexane/EtOAc/NEt₃ v/v/v 90:8:2 (100 mL) to give a light yellow solid (0.17 g) that was purified again by radial chromatography (2 mm silica): hexane/EtOAc/NEt₃ v/v/v 90:8:2 (100 mL), 75:20:5 (100 mL) to give a light yellow solid (0.14 g). A third purification by radial chromatography (2 mm silica): CHCl₃ (150 mL), 1% EtOH/CHCl₃ (50 mL) afforded (R)-Compound 32 (0.13 g, 32%) as an off-white solid: ¹H NMR (400 MHz, CDCl₃) δ 8.39 (s, 2H), 7.92 (dd, 1H, J=10.6 Hz, J=2.4 Hz), 7.46 (dd, 1H, J=8.8 Hz, J=5.4 Hz), 7.01 (td, 1H, J=8.8 Hz, J=2.4 Hz), 6.51 (s, 1H), 5.40 (dt, 1H, ²J_{HF}=52.8 Hz, J=3.2 Hz), 4.03 (ddd, 1H, ³J_{HF}=25.2 Hz, J=13.6 Hz, J=1.6 Hz), 3.93 (t, 1H, J=10.0 Hz), 3.81 (dd, 0.5H, J=13.6 Hz, J=3.6 Hz), 3.76-3.69 (m—overlapping resonances, 1.5 H), 2.46-2.37 (m, 1H), 2.26-2.06 (m, 1H), 1.49 (s, 9H); HRMS (ESI) [M+H]Calcd for C₂₁H₂₃O₂N₄F₂: 401.1784, found: 401.1774.

### tert-Butyl 6-fluoro-2-(2-(4-oxopiperidin-1-yl)pyrimidin-5-yl)-1H-indole-1-carboxylate (Compound 33)

1,4-Dioxane was bubbled with N_{2(g)} for 30 min. Compound 4 (0.24 g, 6.03×10⁻⁴ mol), compound 27 (0.18 g, 7.03×10⁻⁴ mol, 1.2 equiv.), Pd(dppf)Cl₂ (46.5 mg, 6.36×10⁻⁵ mol, 0.1 equiv.), and Na₂CO₃ (0.14 g, 1.32 mmol, 2.2 equiv.) were flushed with N_{2(g)} for 10 min, then 1,4-dioxane (25 mL) was added, the mixture was stirred at reflux under N_{2(g)} for 5 h, then cooled to ambient temperature. The mixture was filtered through Celite, and the Celite was rinsed with EtOAc. The filtrate was concentrated to a dark orange oil, then CH₂Cl₂ and hexane were added and removed to give a dark orange residue that was dissolved in CH₂Cl₂, poured onto dry silica (55 mm h×45 mm i.d.), and eluted under vacuum: hexane/EtOAc/NEt₃ v/v/v 90:8:2 (100 mL), 75:20:5 (100 mL), 50:45:5 (200 mL), 20:75:5 (100 mL) to give a light orange solid (0.134 g). Purification by radial chromatography (2 mm silica): hexane/EtOAc/NEt₃ v/v/v 90:8:2 (100 mL), 75:20:5 (100 mL) gave an off-white solid (79 mg) that was again purified by radial chromatography (2 mm silica): % MeOH/CH₂Cl₂—1% (50 mL), 2% (50 mL) to afford Compound 33 (70 mg, 28%) as an off-white solid: ¹H NMR (300 MHz, CDCl₃) δ 8.42 (s, 2H), 7.93 (dd, 1H, J=10.8 Hz, J=2.4 Hz), 7.47 (dd, 1H, J=8.7 Hz, J=5.4 Hz), 7.02 (td, 1H, J=8.7 Hz, J=2.4 Hz), 6.53 (s, 1H), 4.20 (t, 4H, J=6.3 Hz), 2.54 (t, 4H, J=6.3 Hz), 1.49 (s, 9H); HRMS (ESI) [M+H]Calcd for C₂₂H₂₄O₃N₄F: 411.1827, found: 411.1815.

### tert-Butyl 6-fluoro-2-(6-(piperidin-1-yl)pyridin-3-yl)-1H-indole-1-carboxylate (Compound 34)

1,4-Dioxane (30 mL) was purged with N_{2(g)} for 30 min. Compound 4 (0.44 g, 1.11 mmol), compound 28 (0.29 g, 1.20 mmol, 1.1 equiv), Pd(dppf)Cl₂ (0.10 g, 0.14 mmol, 0.1 equiv), and Na₂CO₃ (0.54 g, 5.09 mmol, 4.6 equiv.) were flushed with N_{2(g)} for 10 min, then 1,4-dioxane was added. The reaction mixture was stirred at reflux under N_{2(g)} for 5 h, then cooled to ambient temperature, filtered through Celite, and the Celite was rinsed with EtOAc. The solvent was removed from the filtrate to give a brown oil, then CH₂Cl₂ and hexane were added and removed to give a brown residue that was dried under vacuum. The residue was dissolved in CH₂Cl₂, poured onto dry silica (55 mm h×45 mm i.d.), and eluted under vacuum: % CH₂Cl₂/hexane—50% (100 mL), 75% (100 mL), CH₂Cl₂ (200 mL), % MeOH/CH₂Cl₂—1% (100 mL), 2.5% (100 mL), 5% (100 mL) to give recovered Compound 4 (0.22 g crude, subsequently purified to give 94 mg, 21% recovery) and a brown residue (0.31 g). The brown residue was dissolved in CH₂Cl₂, poured onto dry silica (55 mm h×45 mm i.d.), and eluted under vacuum: CH₂Cl₂ (100 mL), % MeOH/CH₂Cl₂—1% (100 mL), 2% (100 mL), 3% (200 mL), 5% (200 mL) to give a brown residue (0.27 g) that was purified by radial chromatography (2 mm silica): CHCl₃ (100 mL) to give a light brown solid (0.12 g). Purification by radial chromatography (2 mm silica): hexane/EtOAc/NEt₃ v/v/v 90:8:2 (100 mL), 75:20:5 (25 mL) afforded Compound 34 (89 mg, 20%) as an off-white solid: ¹H NMR (300 MHz, CDCl₃) δ 8.22 (d, 1H, J=2.4 Hz), 7.92 (dd, 1H, J=10.8 Hz, J=2.4 Hz), 7.45 (m, 2H), 6.99 (td, 1H, J=8.7 Hz, 2.4 Hz), 6.67 (d, 1H, J=8.7 Hz), 6.46 (s, 1H), 3.59 (m, 4H), 1.67 (m, 6H), 1.43 (s, 9H); HRMS (ESI) [M+H]⁺ Calcd for C₂₃H₂₇FN₃O₂: 396.2082, found: 396.2081.

### tert-Butyl 6-fluoro-2-(6-morpholinopyridin-3-yl)-1H-indole-1-carboxylate (Compound 35)

Compound 4 (0.41 g, 1.03 mmol), compound 29 (0.26 g, 1.07 mmol, 1 equiv.), Pd(dppf)Cl₂ (81 mg, 0.11 mmol, 0.1 equiv.), Na₂CO₃ (0.32 g, 3.02 mmol, 2.9 equiv.) and 1,4-dioxane (25 mL) were stirred at reflux under N_{2(g)} for 6 h, then cooled to ambient temperature. The reaction mixture was filtered through Celite, the Celite was rinsed with EtOAC, the filtrate was concentrated to a brown oil, then CH₂Cl₂ and hexane were added and removed to give a brown residue that was dried under vacuum. The residue was dissolved in CH₂Cl₂, poured onto dry silica (55 mm h×45 mm i.d.), and eluted under vacuum: hexane/EtOAc/NEt₃ v/v/v 90:8:2 (125 mL), 75:20:5 (100 mL), 50:45: (150 mL), 20:75:5 (200 mL) to give a yellow syrup (0.14 g). Purification by radial chromatography (2 mm silica): hexane/EtOAc/NEt₃ v/v/v 90:8:2 (125 mL), 75:20:5 (100 mL) afforded Compound 35 (66 mg, 16%) as a light yellow foam: ¹H NMR (400 MHz, CDCl₃) δ 8.26 (d, 1H, J=2.0 Hz), 7.91 (dd, 1H, J=10.6 Hz, J=2.0 Hz), 7.54 (dd, 1H, J=8.8 Hz, J=2.4 Hz), 7.45 (dd, 1H, J=8.8 Hz, J=5.4 Hz), 7.00 (td, 1H, J=8.8 Hz, J=2.4 Hz), 6.67 (d, 1H, J=8.8 Hz), 6.48 (s, 1H), 3.85 (t, 4H, J=4.8 Hz), 3.56 (t, 4H, J=4.8 Hz), 1.44 (s, 9H); HRMS (ESI) [M+H]⁺ Calcd for C₂₂H₂₅FN₃O₃: 398.1875, found: 398.1866.

### 6-Fluoro-2-(2-(4-methoxypiperidin-1-yl)pyrimidin-5-yl)-1H-indole (Compound 36)

Crude Compound 36 (91 mg—from coupling reaction of Compound 31 above) was purified by radial chromatography (1 mm silica): CH₂Cl₂ (50 mL), % MeOH/CH₂Cl₂—1% (50 mL), 2% (75 mL) to afford Compound 36 (46 mg, 9%) as a tan solid: ¹H NMR (300 MHz, CDCl₃) δ 8.57 (s, 2H), 8.23 (br s, 1H), 7.50 (dd, 1H, J=8.7 Hz, J=5.4 Hz), 7.08 (dd, 1H, J=9.6 Hz, J=2.1 Hz), 6.89 (ddd, 1H, J=9.7 Hz, J=8.7 Hz, J=2.4 Hz), 6.63 (d, 1H, J=1.5 Hz), 4.31 (m, 2H), 3.50 (m, 3H), 3.41 (s, 3H), 1.97 (m, 2H), 1.62 (m, 2H); ¹³C NMR (125 MHz, DMSO-d₆) δ 160.04, 158.74 (d, ¹J_{FC}=234.4 Hz), 154.52, 136.70 (d, J_{FC}=12.7 Hz), 134.06 (d, J_{FC}=3.5 Hz), 125.42, 120.51 (d, J_{FC}=10.2 Hz), 114.81, 107.76 (d, J_{FC}=24.4 Hz), 97.08 (d, J_{FC}=25.7 Hz), 97.05, 75.49, 54.97, 40.97, 30.20; ¹⁹F NMR (470.6 MHz, DMSO-d₆) δ −121.53 (m).

Compound 36 from N-Boc deprotection of Compound 31:

Compound 31 (64.5 mg, 1.51×10⁻⁴ mol) and TFA (1.5 mL, 19.5 mmol, 129 equiv.) were stirred at ambient temperature for 15 min, then poured into a mixture of NaHCO₃ (1.84 g, 21.90 mmol, 1.1 equiv. TFA), H₂O (45 mL), and CH₂Cl₂ (45 mL). The mixture was stirred, then the layers were separated, and the H₂O layer was extracted with CH₂Cl₂ (10 mL). The combined CH₂Cl₂ layers were washed with brine (20 mL) and dried over MgSO₄. The solution was concentrated, poured onto dry silica (33 mm h×33 mm i.d.), and eluted under vacuum: hexane/EtOAc/NEt₃ v/v/v 90:8:2 (50 mL), 75:20:5 (50 mL), 50:45:5 (100 mL) to afford Compound 36 (30 mg, 61%) as a light yellow solid: H NMR (300 MHz, CDCl₃) δ 8.57 (s, 2H), 8.24 (br s, 1H), 7.49 (dd, 1H, J=8.7 Hz, J=5.4 Hz), 7.07 (dd, 1H, J=9.3 Hz, J=2.1 Hz), 6.89 (ddd, 1H, J=9.6 Hz, J=8.7 Hz, J=2.4 Hz), 6.62 (partially resolved dd, 1H, J=2.1 Hz, J=0.6 Hz), 4.31 (m, 2H), 3.50 (m, 3H), 3.41 (s, 3H), 1.97 (m, 2H), 1.62 (m, 2H); HRMS (ESI) [M+H]⁺ Calcd for C₁₈H₂₀FN₄O: 327.1616, found: 327.1620.

### (R)-6-Fluoro-2-(2-(3-fluoropyrrolidin-1-yl)pyrimidin-5-yl)-1H-indole ((R)-Compound 37)

(R)-Compound 32 (0.12 g, 3.00×10⁻⁴ mol) was dissolved in TFA (3 mL, 38.9 mmol, 130 equiv.), stirred at ambient temperature for 20 min, then poured into a mixture of NaHCO₃ (3.95 g, 47.02 mmol, 1.2 equiv. TFA), H₂O (75 mL), and CH₂Cl₂ (75 mL). The mixture was stirred for 20 min, then the layers were separated, and the aqueous layer was extracted with CH₂Cl₂ (25 mL×2). The combined CH₂Cl₂ layers were washed with brine (25 mL), dried over MgSO₄, and concentrated (precipitate formed). MeOH was added to redissolve the precipitate, then the solution was poured onto dry silica (55 mm h×45 mm i.d.) and eluted under vacuum: % MeOH/CH₂Cl₂—1% (100 mL), 2.5% (100 mL), 5% (200 mL), 10% (50 mL) to give a yellow solid (85 mg). The solid was placed in a medium-fritted filter, rinsed with 1:1 v/v CH₂Cl₂/hexane (1 mL×7), and dried under vacuum to afford (R)-Compound 37 (67 mg, 74%) as a light yellow solid: ¹H NMR (300 MHz, acetone-d₆) δ 10.71 (br s, 1H), 8.80 (s, 2H), 7.52 (dd, 1H, J=8.4 Hz, J=5.4 Hz), 7.10 (dd, 1H, J=9.9 Hz, J=2.1 Hz), 6.84 (ddd, 1H, J=9.9 Hz, J=8.7 Hz, J=2.4 Hz), 6.79 (s, 1H), 5.45 (dm, 1H, ²J_{HF}=52.8 Hz), 4.01-3.58 (m, 4H), 2.37-2.12 (m, 2H); HRMS (ESI) [M+H]⁺ Calcd for C₁₆H₁₅N₄F₂: 301.1259, found: 301.1253.

### 1-(5-(6-Fluoro-1H-indol-2-yl)pyrimidin-2-yl)piperidin-4-one (Compound 38)

Compound 33 (65 mg, 1.58×10⁻⁴ mol) was dissolved in TFA (1.6 mL, 20.8 mmol, 131 equiv.), stirred at ambient temperature for 20 min, then poured into a mixture of NaHCO₃ (2.13 g, 25.36 mmol, 1.2 equiv. TFA), H₂O (40 mL), and CH₂Cl₂ (40 mL). The mixture was stirred for 10 min, then the layers were separated, and the aqueous layer was extracted with CH₂Cl₂ (15 mL×2). The combined CH₂Cl₂ layers were washed with brine (25 mL), dried over MgSO₄, concentrated, poured onto dry silica (55 mm h×45 mm i.d.), and eluted under vacuum: % MeOH/CH₂Cl₂—1% (100 mL), 2.5% (100 mL), 5% (200 mL), 10% (50 mL) to give an off-white solid (43 mg). Purification by radial chromatography (1 mm silica): % MeOH/CH₂Cl₂—1% (50 mL), 2.5% (25 mL) afforded Compound 38 (38 mg, 77%) as a light tan solid: ¹H NMR (300 MHz, CDCl₃) δ 8.65 (s, 2H), 8.27 (br s, 1H), 7.52 (dd, 1H, J=8.7 Hz, J=5.4 Hz), 7.10 (dd, 1H, J=9.3 Hz, J=2.1 Hz), 6.91 (ddd, 1H, J=9.6 Hz, J=8.7 Hz, J=2.1 Hz), 6.68 (dd, 1H, J=2.1 Hz, J=0.6 Hz), 4.20 (t, 4H, J=6.0 Hz), 2.55 (t, 4H, J=6.0 Hz); HRMS (ESI) [M+H]⁺ Calcd for C₁₇H₁₆FN₄O: 311.1303, found: 311.1296. X-ray quality crystals were grown by slow evaporation of acetone.

### 6-fluoro-2-(6-(piperidin-1-yl)pyridin-3-yl)-1H-indole (Compound 39)

Compound 34 (82 mg, 2.07×10⁻⁴ mol) was dissolved in TFA (2 mL, 26 mmol, 126 equiv.), stirred at ambient temperature for 20 min, then poured into a mixture of Na₂CO₃ (2.43 g, 28.93 mmol, 1.1 equiv. TFA), H₂O (60 mL), and CH₂Cl₂ (60 mL). The mixture was stirred for 10 min, then the layers were separated and the aqueous layer was extracted with CH₂Cl₂ (10 mL×3). The combined CH₂Cl₂ layers were washed with brine (30 mL), dried over MgSO₄, then poured onto dry silica (33 mm h×33 mm i.d.), and eluted under vacuum: % MeOH/CH₂Cl₂—1% (50 mL), 2% (75 mL), 3% (50 mL) to give an off-white solid (58 mg). Purification by radial chromatography (1 mm silica): hexane/EtOAc/NEt₃ v/v/v 90:8:2 (40 mL), 75:20:5 (50 mL) afforded Compound 39 (50 mg, 82%) as an off-white solid: ¹H NMR (300 MHz, CDCl₃) δ 8.46 (d, 1H, J=2.4 Hz), 8.28 (br s, 1H), 7.68 (dd, 1H, J=9.0 Hz, J=2.4 Hz), 7.48 (dd, 1H, J=8.7 Hz, J=5.4 Hz), 7.06 (dd, 1H, J=9.6 Hz, J=1.8 Hz), 6.86 (ddd, 1H, J=9.6 Hz, J=8.7 Hz, J=2.1 Hz), 6.70 (d, 1H, J=8.7 Hz), 6.61 (d, 1H, J=1.5 Hz), 3.59 (m, 4H), 1.67 (m, 6H); ¹³C NMR (125 MHz, DMSO-d₆) δ 158.57 (d, ¹J_{FC}=233.9 Hz), 157.98, 144.42, 136.86 (d, J_{FC}=3.5 Hz), 136.66 (d, J_{FC}=12.6 Hz), 134.13, 125.61, 120.25 (d, J_{FC}=9.9 Hz), 116.64, 107.49 (d, J_{FC}=24.0 Hz), 106.98, 96.95 (d, J_{FC}=25.5 Hz), 96.51, 45.50, 24.99, 24.29; ¹⁹F NMR (470.6 MHz, DMSO-d₆) δ −122.04 (m); HRMS (ESI) [M+H]⁺ Calcd for C₁₈H₁₉FN₃: 296.1558, found: 296.1568.

### 4-(5-(6-Fluoro-1H-indol-2-yl)pyridin-2-yl)morpholine (Compound 40)

Compound 35 (61 mg, 1.53×10⁻⁴ mol) was dissolved in TFA (1.5 mL, 19.5 mmol, 127 equiv.), stirred at ambient temperature for 20 min, then poured into a mixture of Na₂CO₃ (1.82 g, 21.67 mmol, 1.1 equiv. TFA), H₂O (40 mL), and CH₂Cl₂ (40 mL). The mixture was stirred for 15 min, then the layers were separated, and the aqueous layer was extracted with CH₂Cl₂ (15 mL×2). The combined CH₂Cl₂ layers were washed with brine (25 mL), dried over MgSO₄, then poured onto dry silica (33 mm h×33 mm i.d.), and eluted under vacuum: % MeOH/CH₂Cl₂—1% (50 mL), 2% (75 mL), 3% (50 mL) to afford Compound 40 (42 mg, 92%) as a light tan solid: ¹H NMR (300 MHz, CDCl₃) δ 8.50 (d, 1H, J=2.1 Hz), 8.28 (br s, 1H), 7.75 (dd, 1H, J=8.7 Hz, J=2.4 Hz), 7.49 (dd, 1H, J=8.7 Hz, J=5.4 Hz), 7.07 (dd, 1H, J=9.3 Hz, J=2.1 Hz), 6.88 (ddd, 1H, J=9.6 Hz, J=8.7 Hz, J=2.1 Hz), 6.71 (d, 1H, J=8.7 Hz), 6.65 (dd, 1H, J=2.1 Hz, J=0.6 Hz), 3.85 (t, 4H, J=4.8 Hz), 3.57 (t, 4H, J=4.8 Hz); ¹³C NMR (125 MHz, DMSO-d₆) δ 158.66 (d, ¹J_{FC}=234.3 Hz), 158.19, 144.28, 136.72 (d, J_{FC}=12.7 Hz), 136.54 (d, J_{FC}=3.4 Hz), 134.19, 125.55, 120.41 (d, J_{FC}=10.1 Hz), 117.95, 107.60 (d, J_{FC}=24.3 Hz), 106.92, 97.00 (d, J_{FC}=25.5 Hz), 96.99, 65.91, 45.04; ¹⁹F NMR (470.6 MHz, DMSO-d₆) δ −121.79; HRMS (ESI) [M+H]⁺ Calcd for C₁₇H₁₇FN₃O: 298.1350, found: 298.1345.

### 1-(5-(5-Fluorobenzofuran-2-yl)pyrimidin-2-yl)piperidin-4-ol (Compound 43)

1,4-Dioxane (30 mL) and H₂O (6 mL) were combined and purged with N_{2(g)} for 45 min. 5-Fluorobenzofuran-2-boronic acid (Compound 41) (0.25 g, 1.39 mmol), compound 26 (0.42 g, 1.63 mmol, 1.2 equiv.), Pd(dppf)Cl₂·CH₂Cl₂ (97 mg, 1.19×10⁻⁴ mol, 0.09 equiv.), and Na₂CO₃ (0.71 g, 6.70 mmol, 4.8 equiv.) were flushed with N_{2(g)} for 10 min, then the H₂O/1,4-dioxane mixture (17 mL) was added. The reaction mixture was stirred under N_{2(g)} in a heated sand bath (~95° C.) for 14 h, then cooled to ambient temperature, filtered through Celite, and the Celite was rinsed with EtOAc. The filtrate was concentrated, then EtOAc was added and removed to give a brown residue that was dissolved in CH₂Cl₂, poured onto dry silica (55 mm h×45 mm i.d.), and eluted under vacuum: hexane/EtOAc/NEt₃ v/v/v 75:20:5 (100 mL), 50:45:5 (150 mL), 20:75:5 (250 mL), EtOAc (50 mL). The desired fractions were combined and purified by radial chromatography (2 mm silica): % EtOH/CHCl₃—1% (50 mL), 2% (75 mL), 3% (100 mL) to give a tan solid (77 mg). The solid was suspended in CHCl₃ (2 mL), filtered, rinsed with CHCl₃ (1.5 mL×2) and dried under vacuum to afford Compound 43 (47 mg, 11%) as a white solid: ¹H NMR (300 MHz, DMSO-d₆) δ 8.85 (s, 2H), 7.60 (dd, 1H, J=9.0 Hz, J=4.2 Hz), 7.43 (dd, 1H, J=9.0 Hz, J=2.4 Hz), 7.26 (s, 1H), 7.10 (td, 1H, J=9.0 Hz, J=2.4 Hz), 4.77 (d, 1H, J=4.2 Hz), 4.30 (partially resolved dddd, 2H, J=13.5 Hz, J=4.5 Hz), 3.77 (apparent octet, 1H, J=4.2 Hz), 3.39 (ddd—partially obscured by H₂O resonance, 2H, J=9.6 Hz, J=3.3 Hz), 1.80 (m, 2H), 1.36 (m, 2H); HRMS (ESI) [M+H]⁺ Calcd for C₁₇H₁₇FN₃O₂: 314.1299, found: 314.1296.

### tert-Butyl 2-(2-(4-hydroxypiperidin-1-yl)pyrimidin-5-yl)-1H-indole-1-carboxylate (Compound 44)

1,4-Dioxane (10 mL) and H₂O (2 mL) were combined and purged with N_{2(g)} for 10 min. Compound 26 (0.24 g, 9.30×10⁻⁴ mol), Boc-indole-2-boronic acid (Compound 6) (0.26 g, 9.96×10⁻⁴ mol, 1.1 equiv.), Pd(dppf)Cl₂ (94 mg, 1.15×10⁻⁴ mol, 0.1 equiv.), and Na₂CO₃ (0.45 g, 4.25 mmol, 4.6 equiv.) were flushed with N_{2(g)} for 15 min, then the H₂O/1,4-dioxane mixture was added. The reaction mixture was stirred under N_{2(g)} in a heated sand bath (85° C.) for 16 h, then cooled to ambient temperature, filtered through Celite, and the Celite was rinsed with EtOAc. The solvent was removed to give a black syrup that was dissolved in CH₂Cl₂, poured onto dry silica (45 mm h×45 mm i.d.), and eluted under vacuum: CH₂Cl₂ (100 mL), % MeOH/CH₂Cl₂—1% (100 mL), 2.5% (100 mL), 5% (100 mL). The desired fractions were combined, concentrated, poured onto dry silica (45 mm h×45 mm i.d.), and eluted under vacuum: hexane/EtOAc/NEt₃ v/v/v 90:8:2 (50 mL), 75:20:5 (100 mL), 50:45:5 (100 mL), 20:75:5 (200 mL) to give an orange/tan foam (0.25 g). Purification by radial chromatography (2 mm silica): CHCl₃ (50 mL), % EtOH/CHCl₃—1% (50 mL), 2% (75 mL), 3% (50 mL) afforded Compound 44 (0.23 g, 63%) as a light-yellow foam: ¹H NMR (400 MHz, CDCl₃) δ 8.36 (s, 2H), 8.19 (d, 1H, J=8.4 Hz), 7.54 (d, 1H, J=6.0 Hz), 7.32 (apparent t, 1H, J=7.6 Hz), 7.25 (apparent t—partially obscured by CHCl₃ resonance, 1H, J=7.6 Hz), 6.53 (s, 1H), 4.46 (partially resolved dddd, 2H, J=13.6 Hz, J=4.4 Hz), 3.99 (br s, 1H), 3.40 (partially resolved ddd, 2H, J=11.4 Hz, J=2.8 Hz), 1.98 (m, 2H), 1.57 (m, 2H), 1.48 (s, 10H).

### tert-Butyl 5-fluoro-2-(2-(4-hydroxypiperidin-1-yl)pyrimidin-5-yl)-1H-indole-1-carboxylate (Compound 45)

1,4-Dioxane (30 mL) and H₂O (6 mL) were combined and purged with N_{2(g)} for 45 min. N-Boc-5-fluoroindole-2-boronic acid (Compound 42) (0.25 g, 8.96×10⁻⁴ mol), compound 26 (0.25 g, 9.69×10⁻⁴ mol, 1.1 equiv.), Pd(dppf)Cl₂·CH₂Cl₂ (79 mg, 9.67×10⁻⁵ mol, 0.1 equiv.), and Na₂CO₃ (0.47 g, 4.43 mmol, 5 equiv.) were flushed with N_{2(g)} for 10 min, then the H₂O/1,4-dioxane mixture (16 mL) was added. The reaction mixture was stirred under N_{2(g)} in a heated sand bath (~95° C.) for 14 h, then cooled to ambient temperature, filtered through Celite, and the Celite was rinsed with EtOAc. The filtrate was concentrated to an oil, then EtOAc was added and removed to give a brown residue that was dissolved in CH₂Cl₂, poured onto dry silica (55 mm h×45 mm i.d.), and eluted under vacuum: hexane/EtOAc/NEt₃ v/v/v 75:20:5 (100 mL), 50:45:5 (150 mL), 20:75:5 (200 mL) to give a tan foam (0.23 g). Purification by radial chromatography (2 mm silica): % EtOH/CHCl₃—1% (50 mL), 2% (75 mL) afforded Compound 45 (0.17 g, 46%) as an off-white foam: ¹H NMR (400 MHz, CDCl₃) δ 8.35 (s, 2H), 8.14 (dd, 1H, J=8.8 Hz, J=4.8 Hz), 7.19 (dd, 1H, J=8.8 Hz, J=2.4 Hz), 7.04 (td, 1H, J=9.2 Hz, J=2.4 Hz), 6.49 (s, 1H), 4.46 (partially resolved dddd, 2H, J=13.6 Hz, J=4.4 Hz), 4.00 (apparent octet, 1H, J=4.4 Hz), 3.40 (ddd, 1H, J=10.0 Hz, J=3.2 Hz), 1.98 (m, 2H), 1.56 (m, 3H), 1.47 (s, 9H).

### 1-(5-(1H-Indol-2-yl)pyrimidin-2-yl)piperidin-4-ol (Compound 46)

Compound 44 (83 mg, 2.10×10⁻⁴ mol) was dissolved in TFA (2 mL, 26 mmol, 124 equiv.), stirred at ambient temperature for 20 min, then poured into a mixture of NaHCO₃ (2.40 g, 28.57 mmol, 1.1 equiv. TFA), H₂O (60 mL), and CH₂Cl₂ (60 mL). The mixture was stirred until gas evolution ceased, then the layers were separated, and the aqueous layer was extracted with CH₂Cl₂ (10 mL). The combined CH₂Cl₂ layers were washed with brine (25 mL), dried over MgSO₄, and concentrated which produced a precipitate. MeOH was added (several drops) until the precipitate dissolved, then the solution was poured onto dry silica (55 mm h×45 mm i.d.), and eluted under vacuum: % MeOH/CH₂Cl₂—1% (100 mL), 2.5% (100 mL), 5% (150 mL), 10% (50 mL) to give an off-white solid. The solid was dissolved/suspended in CHCl₃ (~4 mL) then MeOH (-0.5 mL) was added to get the remaining solid to dissolve. The solution was purified by radial chromatography (2 mm silica): CHCl₃ (25 mL), % EtOH/CHCl₃—1% (50 mL), 2% (75 mL), 3% (50 mL), 4% (50 mL), 10% (60 mL) to afford Compound 46 (58 mg, 94%) as an off-white solid: ¹H NMR (400 MHz, acetone-d₆) δ 10.59 (br s, 1H), 8.80 (s, 2H), 7.54 (d, 1H, J=7.6 Hz), 7.37 (d, 1H, J=8.4 Hz), 7.08 (td, 1H, J=7.6 Hz, J=1.2 Hz), 7.01 (td, 1H, J=7.6 Hz, J=1.2), 6.78 (d, 1H, J=1.2 Hz), 4.42 (partially resolved dddd, 2H, J=13.6 Hz, J=4.4 Hz), 3.91 (apparent octet, 1H, J=4.4 Hz), 3.84 (d, 1H, J=4.4 Hz), 3.42 (ddd, 2H, J=13.4 Hz, J=9.8 Hz, J=3.2 Hz), 1.90 (m, 2H), 1.48 (m, 2H); HRMS (ESI) [M+H]⁺ Calcd for C₁₇H₁₉N₄O: 295.1553, found: 295.1553.

### 1-(5-(5-Fluoro-1H-indol-2-yl)pyrimidin-2-yl)piperidin-4-ol (Compound 47)

Compound 45 (90 mg, 2.18×10⁻⁴ mol) was dissolved in TFA (2 mL, 26 mmol, 119 equiv.), stirred at ambient temperature for 20 min, then poured into a mixture of NaHCO₃ (2.45 g, 29.16 mmol, 1.1 equiv. TFA), H₂O (60 mL), and CH₂Cl₂ (60 mL). The mixture was stirred for 20 min, the layers were separated, and the aqueous layer was extracted with CH₂Cl₂ (10 mL). The combined CH₂Cl₂ layers were washed with brine (25 mL) and dried over MgSO₄. The solution was concentrated, poured onto dry silica (55 mm h×45 mm i.d.), and eluted under vacuum: CH₂Cl₂ (50 mL), % MeOH/CH₂Cl₂—1% (100 mL), 2.5% (100 mL), 5% (100 mL), 10% (100 mL) to give a faint-yellow solid (54 mg). Purification by radial chromatography (2 mm silica): hexane/EtOAc/NEt₃ v/v/v 75:20:5 (100 mL), 50:45:5 (100 mL), 20:75:5 (250 mL) afforded Compound 47 (47 mg, 69%) as a light-tan solid: ¹H NMR (300 MHz, acetone-d₆) δ 10.67 (br s, 1H), 8.79 (s, 2H), 7.36 (dd, 1H, J=8.7 Hz, J=5.4 Hz), 7.23 (dd, 1H, J=9.6 Hz, J=2.7 Hz), 6.87 (ddd, 1H, J=9.6 Hz, J=8.7 Hz, J=2.7 Hz), 6.77 (m, 1H), 4.41 (partially resolved dddd, 2H, J=13.5 Hz, J=4.5 Hz), 3.92 (apparent octet, 1H, J=4.2 Hz), 3.83 (d, 1H, J=4.2 Hz), 3.43 (ddd, 2H, J=13.2 Hz, J=9.6 Hz, J=3.3 Hz), 1.90 (m, 2H), 1.48 (dddd, 2H, J=13.2 Hz, J=9.0 Hz, J=4.2 Hz); HRMS (ESI) [M+H]⁺ Calcd for C₁₇H₁₈FN₄O: 313.1459, found: 313.1456.

### 2-Fluoro-6-(piperidin-1-yl)pyridine (Compound 48)

2,6-Difluoropyridine (1.34 g, 11.64 mmol), piperidine (1.2 mL, 12.1 mmol, 1 equiv.), i-Pr₂NEt (3.2 mL, 18.4 mmol, 1.6 equiv.), and 1,4-dioxane (10 mL) were stirred at reflux under N_{2(g)} for 90 min, then cooled to ambient temperature. The reaction mixture was concentrated to a yellow oil, CH₂Cl₂ and hexane were added, the solution was poured onto dry silica (55 mm h×45 mm i.d.), and eluted under vacuum: hexane (100 mL), % CH₂Cl₂/hexane—10% (100 mL), 25% (200 mL), 50% (100 mL), 75% (100 mL), CH₂Cl₂ (100 mL) to afford Compound 48 (1.50 g, 71%) as a colorless oil: ¹H NMR (300 MHz, CDCl₃) δ 7.48 (dd, 1H, J_{HF}=16.7 Hz, J=8.1 Hz), 6.40 (dd, 1H, J=8.1 Hz, J=2.7 Hz), 6.09 (dd, 1H, J=7.8 Hz, J=3.0 Hz), 3.51 (m, 4H), 1.64 (m, 6H); ¹⁹F NMR (282.5 MHz, CDCl₃) δ −68.60 (d, J=6.5 Hz); ¹³C NMR (125 MHz, CDCl₃) δ 162.96 (d, ¹J_{CF}=233.0 Hz), 158.58 (d, ³J_{CF}=15.9 Hz), 141.73 (d, ³J_{CF}=8.4 Hz), 102.69 (d, ⁴J_{CF}=4.0 Hz), 94.86 (d, ²J_{CF}=37.6 Hz), 46.12, 25.46, 24.70); HRMS (ESI) [M+H]⁺ Calcd for C₁₀H₁₄FN₂: 181.1136, found: 181.1133.

### 4-(6-Fluoropyridin-2-yl)morpholine (Compound 49)

2,6-Difluoropyridine (1.34 g, 11.64 mmol), morpholine (1 mL, 11.6 mmol, 1 equiv.), i-Pr₂NEt (3 mL, 17.2 mmol, 1.5 equiv.), and 1,4-dioxane (10 mL) were stirred at reflux under N_{2(g)} for 90 min, cooled to ambient temperature, and concentrated to an oil that was dried under vacuum to give an off-white solid. The solid was dissolved in CH₂Cl₂, poured onto dry silica (55 mm h×45 mm i.d.), and eluted under vacuum: hexane (50 mL), % CH₂Cl₂/hexane—25% (50 mL), 50% (75 mL), 75% (100 mL), CH₂Cl₂ (500 mL), % MeOH/CH₂Cl₂—2% (75 mL), 5% (50 mL), 10% (100 mL) to afford Compound 49 (0.72 g, 34%) as a colorless oil that slowly became a white solid: ¹H NMR (300 MHz, CDCl₃) δ 7.55 (dd, 1H, J_{CF}=16.5 Hz, J=8.1 Hz), 6.41 (dd, 1H, J=8.1 Hz, J=2.7 Hz), 6.21 (dd, 1H, J=7.8 Hz, J=2.7 Hz), 3.80 (t, 4H, J=4.8 Hz), 3.50 (t, 4H, J=4.8 Hz); ¹⁹F NMR (282.5 MHz, CDCl₃) δ −68.47 (d, J=7.1 Hz); ¹³C NMR (125 MHz, CDCl₃) δ 162.87 (d, ¹J_{CF}=234.6 Hz), 158.64 (d, ³J_{CF}=15.5 Hz), 142.07 (d, ³J_{CF}=8.3 Hz), 102.74 (d, ⁴J_{CF}=4.0 Hz), 96.74 (d, ²J_{CF}=37.1 Hz), 66.65, 45.40; HRMS (ESI) [M+H]⁺ Calcd for C₉H₁₂FN₂O: 183.0928, found: 183.0926.

### 2-nitro-6-(piperidin-1-yl)pyridine (Compound 50)

2-Chloro-6-nitropyridine (0.28 g, 1.77 mmol), piperidine (0.2 mL, 2.0 mmol, 1.1 equiv.), i-Pr₂NEt (0.5 ml, 2.9 mmol, 1.6 equiv.), and 1,4-dioxane (5 mL) were stirred at reflux under N_{2(g)} for 2 h, then cooled to ambient temperature. The reaction mixture was poured onto dry silica (55 mm h×45 mm i.d.), and eluted under vacuum: hexane (50 mL), hexane/EtOAc/NEt₃ v/v/v 90:8:2 (100 mL), 75:20:5 (150 mL) to give a yellow/orange syrup (0.27 g). Purification by radial chromatography (2 mm silica): hexane/EtOAc/NEt₃ v/v/v 90:8:2 (100 mL), 75:20:5 (25 mL) afforded Compound 50 (0.16 g, 44%) as an orange syrup: ¹H NMR (300 MHz, CDCl₃) δ 7.64 (dd, 1H, J=8.4 Hz, J=7.5 Hz), 7.37 (d, 1H, J=7.5 Hz), 6.89 (d, 1H, J=8.4 Hz), 3.63 (m, 4H), 1.67 (m, 6H).

### 4-(6-Nitropyridin-2-yl)morpholine (Compound 51)

2-Chloro-6-nitropyridine (0.28 g, 1.77 mmol), morpholine (0.2 mL, 2.3 mmol, 1.3 equiv.), i-Pr₂NEt (0.5 ml, 2.9 mmol, 1.6 equiv.), and 1,4-dioxane (5 mL) were stirred at reflux under N_{2(g)} for 4 h, then cooled to ambient temperature, filtered, and the precipitate was rinsed with EtOAc. The solvent was removed from the filtrate to give an orange residue that was dissolved in CH₂Cl₂, poured onto dry silica (55 mm h×45 mm id.), and eluted under vacuum: CH₂Cl₂ (200 mL), % MeOH/CH₂Cl₂—1% (100 mL), 2.5% (100 mL), 5% (150 mL) to give a yellow/orange solid. Purification by radial chromatography (2 mm silica): hexane/EtOAc/NEt₃ v/v/v 90:8:2 (100 mL), 75:20:5 (75 mL) afforded Compound 51 (0.14 g, 38%) as a yellow/orange solid: ¹H NMR (300 MHz, CDCl₃) δ 7.73 (dd, 1H, J=8.4 Hz, J=7.5 Hz), 7.49 (d, 1H, J=7.5 Hz), 6.90 (d, 1H, J=8.4 Hz), 3.83 (t, 4H, J=5.1 Hz), J=3.63 (t, 4H, J=5.1 Hz); ¹³C NMR (125 MHz, CDCl₃) δ 158.17, 156.06, 140.49, 111.79, 106.21, 66.67, 45.27; HRMS (ESI) [M+H]⁺ Calcd for C₉H₁₂N₃O₃: 210.0873, found: 210.0871.

### 3-Bromo-2-fluoro-6-(piperidin-1-yl)pyridine (Compound 52)

Compound 48 (0.77 g, 4.27 mmol) was dissolved in CH₃CN (25 mL) under N_{2(g)} and cooled to 0° C. NBS (0.86 g, 4.83 mmol, 1.1 equiv.) was added, the reaction mixture was stirred at 0° C. for 5 min, then warmed to ambient temperature and stirred for 6 h. The CH₃CN was removed to give a light tan residue that was dissolved in CH₂Cl₂, poured onto dry silica (55 mm h×45 mm id.), and eluted under vacuum with CH₂Cl₂ (150 mL) to give a colorless oil (1.26 g). Purification by radial chromatography (2 mm silica): % CH₂Cl₂/hexane—10% (100 mL), 25% (50 mL) afforded Compound 52 (0.78 g, 70%) as a colorless oil: ¹H NMR (300 MHz, CDCl₃) δ 7.57 (apparent t, 1H, J=8.7 Hz), 6.33 (dd, 1H, J=8.7 Hz, J=1.5 Hz), 3.49 (m, 4H), 1.62 (m, 6H); ¹⁹F NMR (470.6 MHz, CDCl₃) δ −66.52 (d, J=8.5 Hz); ¹³C NMR (125 MHz, CD30D) δ 159.43 (d, ¹J_{CF}=229.3 Hz), 158.53 (d, ³J_{CF}=14.9 Hz), 145.54 (d, ³J_{CF}=2.4 Hz), 106.08 (d, ⁴J_{CF}=4.4 Hz), 87.26 (d, ².J_{CF}=38.4 Hz), 47.17, 26.48, 25.66; HRMS (ESI) [M+H]⁺ Calcd for C₁₀H₁₃⁷⁹BrFN₂: 259.0241, found: 259.0238.

### 4-(5-Bromo-6-fluoropyridin-2-yl)morpholine (Compound 53)

Compound 49 (0.36 g, 1.98 mmol) was dissolved in CH₃CN (12 mL) under N_{2(g)} and cooled to 0° C. NBS (0.39 g, 2.19 mmol, 1.1 equiv.) was added, the reaction mixture was stirred at 0° C. for 5 min, then warmed to ambient temperature and stirred for 6 h. The CH₃CN was removed to give a red/brown syrup that was dissolved in CH₂Cl₂, poured onto dry silica (45 mm h×45 mm i.d.), and eluted under vacuum with CH₂Cl₂ (300 mL), % MeOH/CH₂Cl₂—1% (100 mL), 2% (100 mL) to give a white solid (0.54 g). Purification by radial chromatography (2 mm silica): % CH₂Cl₂/hexane—25% (100 mL), 50% (100 mL), 75% (100 mL) afforded Compound 53 (0.28 g, 54%) as a white solid: ¹H NMR (300 MHz, CDCl₃) δ 7.65 (apparent t, 1H, J=8.7 Hz), 6.34 (dd, 1H, J=8.7 Hz, J=1.5 Hz), 3.79 (t, 4H, J=4.8 Hz), 3.48 (t, 4H, J=4.8 Hz); ¹⁹F NMR (470.6 MHz, CDCl₃) δ −66.22 (d, J=8.5 Hz); ¹³C NMR (125 MHz, CDCl₃) δ 158.35 (d, ¹J_{CF}=241.5 Hz), 157.37 (d, ³J_{CF}=23.8 Hz), 144.64 (d, ³J_{CF}=2.6 Hz), 104.63 (d, ⁴J_{CF}=4.5 Hz), 89.09 (d, ²J_{CF}=38.5 Hz), 66.60, 45.44; HRMS (ESI) [M+H]⁺ Calcd for C₉H₁₁⁷⁹BrFN₂O: 261.0033, found: 261.0063.

### 3-Bromo-2-nitro-6-(piperidin-1-yl)pyridine (Compound 54)

Compound 50 (0.16 g, 7.72×10⁻⁴ mol) was flushed with N_{2(g)}, then dissolved in CH₃CN (10 mL) and cooled to 0° C. NBS (0.14 g, 7.87×10⁻⁴ mol) was added, the reaction mixture was warmed to ambient temperature, and stirred under N_{2(g)} for 5.5 h. The CH₃CN was removed to give an orange syrup, then CH₂Cl₂ and hexane were added and removed to give an orange residue that was dissolved in CH₂Cl₂, poured onto dry silica (45 mm h×45 mm i.d.), and eluted under vacuum: hexane (50 mL), % CH₂Cl₂/hexane—25% (100 mL), 50% (100 mL), CH₂Cl₂ (150 mL) to give a dark yellow syrup. Purification by radial chromatography (2 mm silica): % CH₂Cl₂/hexane—25% (100 mL), 50% (50 mL) afforded Compound 54 (0.15 g, 68%) as a yellow syrup: ¹H NMR (500 MHz, CDCl₃) δ 7.64 (d, 1H, J=9.0 Hz), 6.66 (d, 1H, J=9.0 Hz), 3.54 (t, 4H, J=5.5 Hz), 1.66 (m, 2H), 1.62 (m, 4H); ¹³C NMR (125 MHz, CDCl₃) δ 156.45, 156.14, 144.12, 111.10, 92.85, 46.17, 25.49, 24.52; HRMS (ESI) [M+H]⁺ Calcd for C₁₀H₁₃⁷⁹BrN₃O₂: 286.0186, found: 286.0172.

### 4-(5-Bromo-6-nitropyridin-2-yl)morpholine (Compound 55)

Compound 51 (0.12 g, 5.74×10⁻⁴ mol) was dissolved in CH₃CN (10 mL), cooled to 0° C., and NBS (0.15 g, 8.43×10⁻⁴ mol, 1.5 equiv.) was added. The reaction mixture was stirred in a capped flask at ambient temperature for 6 h, concentrated to an orange oil, then CH₂Cl₂ and hexane were added and removed 2× to give an orange solid that was dried under vacuum. The solid was dissolved in CH₂Cl₂, poured onto dry silica (45 mm h×45 mm i.d.), and eluted under vacuum: CH₂Cl₂ (400 mL), 1% MeOH/CH₂Cl₂ (100 mL) to give a yellow solid (0.13 g). Purification by radial chromatography (2 mm silica) with CHCl₃ (100 mL) afforded Compound 55 (82 mg, 50%) as a yellow solid: ¹H NMR (400 MHz, CDCl₃) δ 7.76 (d, 1H, J=9.2 Hz), 6.68 (d, 1H, J=9.2 Hz), 3.80 (t, 4H, J=4.8 Hz), 3.54 (t, 4H, J=4.8 Hz); ¹³C NMR (125 MHz, CDCl₃) δ 156.65, 156.07, 144.61, 110.99, 94.88, 66.51, 45.21; HRMS (ESI) [M+H]⁺ Calcd for C₉H₁₁⁷⁹BrN₃O₃: 287.9978, found: 287.9981.

### 4-(4-Fluoropyridin-2-yl)morpholine (Compound 56)

2-Bromo-4-fluoropyridine (1.0 g, 5.68 mmol), morpholine (0.50 g, 5.74 mmol), sodium tert-butoxide (0.58 g, 6.04 mmol), toluene (50 mL), Pd₂(dba)₃ (73 mg, 7.97×10⁻⁵ mol) and XantPhos (0.14 g, 2.42×10⁻⁴ mol) were flushed with N_{2(g)} for 10 min, then heated at 100° C. overnight. The reaction mixture was cooled to ambient temperature, filtered through a short silica gel pad, and washed with EtOAc. The filtrate was evaporated to dryness and the residue was purified by radial chromatography (hexane/EtOAc v/v 95:5 to 80:20) to afford Compound 56 (0.88 g, 85%) as white solid: ¹H NMR (CDCl₃, 300 MHz): δ 8.13 (dd, 1H, J=9.4 Hz, J=5.4 Hz), 6.41 (ddd, 1H, J=8.1 Hz, J=5.4 Hz, J=2.1 Hz), 6.28 (dd, 1H, J=12.3 Hz, J=2.1 Hz), 3.81 (apparent t, 4H, J=4.8 Hz), 3.49 (apparent t, 4H, J=4.8 Hz); HRMS (ESI) [M+H]⁺ Calcd for C₉H₁₂ON₂F: 183.0928, found: 183.0931.

### 4-(5-Bromo-4-fluoropyridin-2-yl)morpholine (Compound 57)

Compound 56 (0.87 g, 4.78 mmol) was dissolved in CH₃CN (50 mL), cooled to 0° C., then NBS (0.85 g, 4.78 mmol) was added. The reaction mixture was stirred at 0° C. for 90 min, the solvent was removed, and the residue was purified by flash column chromatography (4:1 v/v hexane/EtOAc) to afford Compound 57 (1.20 g, 96%) as an off-white solid: ¹H NMR (300 MHz, CDCl₃) δ 8.21 (d, 1H, J=9.9 Hz), 6.36 (d, 1H, J=11.5 Hz), 3.80 (apparent t, 4H, J=4.8 Hz), 3.48 (apparent t, 4H, J=4.8 Hz). HRMS (ESI) [M+H]⁺ Calcd for C₉H₁₁ON₂BrF: 261.0033, found: 261.0029.

### 5-Bromo-3-fluoro-2-(piperidin-1-yl)pyridine (Compound 58)

2,5-Dibromo-3-fluoropyridine (0.27 g, 1.06 mmol), piperidine (85 mg, 1.00 mmol), sodium tert-butoxide (144 mg, 1.50 mmol), toluene (20 mL), Pd₂(dba)₃ (18 mg, 1.97×10⁻⁵ mol), and XantPhos (35 mg, 6.05×10⁻⁵ mol) were flushed with N_{2(g)} for 5 min, then heated at 100° C. overnight. The reaction mixture was cooled to ambient temperature, filtered through a short silica gel pad, and washed with EtOAc. The filtrate was evaporated to dryness and the residue was purified by radial chromatography (7:3 v/v hexane/EtOAc) to afford Compound 58 (0.15 g, 58%) as a white solid: H NMR (300 MHz, CDCl₃): δ 8.02 (dd, 1H, J=2.1 Hz, J=0.9 Hz), 7.33 (dd, 1H, J=12.0 Hz, J=2.1 Hz), 3.41 (m, 4H), 1.65 (m, 6H); HRMS (ESI) [M+H]⁺ Calcd for C₁₀H₁₃N₂BrF: 259.0241, found: 259.0237.

### 4-(5-Bromo-3-fluoropyridin-2-yl)morpholine (Compound 59)

2,5-Dibromo-3-fluoropyridine (0.27 g, 1.06 mmol), morpholine (87 mg, 1.00 mmol), sodium tert-butoxide (144 mg, 1.50 mmol), toluene (20 mL), Pd₂(dba)₃ (18 mg, 1.97×10⁻⁵ mol), and XantPhos (35 mg, 6.05×10⁻⁵ mol) were flushed with N_{2(g)} for 5 min, then heated at 100° C. overnight. The reaction mixture was cooled to ambient temperature, filtered through a short silica gel pad, and washed with EtOAc (4×10 mL). The filtrate was evaporated to dryness and the residue was purified by radial chromatography (hexane/EtOAc v/v 95:5 to 80:20) to afford Compound 59 (0.16 g, 61%) as white solid: ¹H NMR (300 MHz, CDCl₃) δ 8.05 (dd, 1H, J=1.8 Hz, J=0.9 Hz), 7.39 (dd, 1H, J=12.0 Hz, J=1.8 Hz), 3.82 (apparent t, 4H, J=4.8 Hz), 3.46 (apparent t, 4H, J=4.8 Hz); HRMS (ESI) [M+H]⁺ Calcd for C₉H₁₁ON₂BrF: 261.0033, found: 261.0044.

### 2-Chloro-3-fluoro-5-(piperidin-1-yl)pyridine (Compound 60)

5-Bromo-2-chloro-3-fluoropyridine (0.27 g, 1.28 mmol), piperidine (88 mg, 1.03 mmol), sodium tert-butoxide (144 mg, 1.50 mmol), toluene (20 mL), Pd₂(dba)₃ (18 mg, 1.97×10⁻⁵ mol), and XantPhos (35 mg, 6.05×10⁻⁵ mol) were flushed with N_{2(g)} for 5 min, then heated at 100° C. overnight. The reaction mixture was cooled to ambient temperature, filtered through a short silica gel pad, and washed with EtOAc. The filtrate was evaporated to dryness and the residue was purified by radial chromatography (7:3 v/v hexane/EtOAc) to afford Compound 60 (0.22 g, 99%) as white solid: H NMR (300 MHz, CDCl₃) δ 7.84 (d, 1H, J=2.7 Hz), 6.95 (dd, 1H, J=11.4 Hz, J=2.7 Hz), 3.20 (m, 4H), 1.67 (m, 6H); HRMS (ESI) [M+H]⁺ Calcd for C₁₀H₁₃N₂ClF: 215.0746, found: 215.0743.

### 4-(6-Chloro-5-fluoropyridin-3-yl)morpholine (Compound 61)

5-Bromo-2-chloro-3-fluoropyridine (0.27 g, 1.28 mmol), morpholine (0.11 g, 1.26 mmol), sodium tert-butoxide (144 mg, 1.50 mmol), toluene (20 mL), Pd₂(dba)₃ (18 mg, 1.97×10⁻⁵ mol), and XantPhos (35 mg, 6.05×10⁻⁵ mol) were flushed with N_{2(g)} for 5 min, then heated at 100° C. overnight. The reaction mixture was cooled to ambient temperature, filtered through a short silica gel pad, and washed with EtOAc. The filtrate was evaporated to dryness and the residue was purified by radial chromatography (7:3 v/v hexane/EtOAc) to afford Compound 61 (0.25 g, 92%) as white solid: H NMR (300 MHz, CDCl₃) δ 7.85 (d, 1H, J=2.7 Hz), 6.97 (dd, 1H, J=10.8 Hz, J=2.7 Hz), 3.87 (m—AA′XX′, 4H, J=4.8 Hz, J=1.8 Hz), 3.19 (m—AA′XX′, 4H, J=4.8 Hz, J=1.8 Hz); HRMS (ESI) [M+H]⁺ Calcd for C₉H₁₁ON₂ClF: 217.0539, found: 217.0536.

### 4-(6-Chloro-4-fluoropyridin-3-yl)morpholine (Compound 62)

2-Chloro-4-fluoro-5-bromopyridine (0.27 g, 1.28 mmol), morpholine (87 mg, 1.00 mmol), sodium tert-butoxide (144 mg, 1.50 mmol), toluene (20 mL), Pd₂(dba)₃ (18 mg, 1.97×10⁻⁵ mol), and XantPhos (35 mg, 6.05×10⁻⁵ mol) were flushed with N_{2(g)} for 5 min, then heated at 100° C. overnight. The reaction mixture was cooled to ambient temperature, filtered through a short silica gel pad, and washed with EtOAc. The filtrate was evaporated to dryness and the residue was purified by radial chromatography (hexane/EtOAc v/v 95:5 to 80:20) to afford Compound 62 (97 mg, 45%) as white solid: ¹H NMR (300 MHz, CDCl₃) δ 7.98 (d, 1H, J=10.5 Hz), 7.04 (d, 1H, J=11.1 Hz), 3.86 (m—AA′XX′, 4H, J=4.8 Hz, J=1.8 Hz), 3.12 (m—AA′XX′, 4H, J=4.8 Hz, J=1.8 Hz); HRMS (ESI) [M+H]⁺ Calcd for C₉H₁₁ON₂ClF: 217.0539, found: 217.0536.

### tert-Butyl 2-(2-fluoro-6-(piperidin-1-yl)pyridin-3-yl)-1H-indole-1-carboxylate (Compound 63)

1,4-Dioxane (30 mL) and H₂O (6 mL) were combined and purged with N_{2(g)} for 30 min. N-Boc-indole-2-boronic acid (Compound 6) (0.25 g, 9.58×10⁻⁴ mol), compound 52 (0.31 g, 1.20 mmol, 1.2 equiv.), Pd(dppf)Cl₂ (95 mg, 1.30×10⁻⁴ mol, 0.14 equiv.), and Na₂CO₃ (0.47 g, 4.43 mmol, 4.6 equiv.) were flushed with N_{2(g)} for 10 min, then the H₂O/1,4-dioxane mixture (15 mL) was added. The reaction mixture was stirred at reflux under N_{2(g)} for 4 h, then cooled to ambient temperature, filtered through Celite, and the Celite was rinsed with 1,4-dioxane, then EtOAc. The solvent was removed from the filtrate to give a brown oil that was dried under vacuum to give a brown residue. The residue was dissolved in CH₂Cl₂, poured onto dry silica (55 mm h×45 mm i.d.), and eluted under vacuum with CH₂Cl₂ (250 mL) to give a faint tan syrup (0.19 g). Purification by radial chromatography (2 mm silica) with 90:8:2 v/v/v hexane/EtOAc/NEt₃ (100 mL) afforded Compound 63 (87 mg, 23%) as a white foam: ¹H NMR (300 MHz, CDCl₃) δ 8.20 (d, 1H, J=8.4 Hz), 7.55 (m, 2H), 7.31 (ddd, 1H, J=8.4 Hz, J=7.2 Hz, J=1.2 Hz), 7.22 (td, 1H, J=7.2 Hz, J=0.9 Hz), 6.53 (s, 1H), 6.49 (dd, 1H, J=8.4 Hz, J=1.8 Hz), 3.58 (m, 4H), 1.66 (m, 6H), 1.45 (s, 9H); ¹⁹F NMR (282.5 MHz, CDCl₃) δ −67.96 (d, J=9.6 Hz); HRMS (ESI) [M+H]⁺ Calcd for C₂₃H₂₇FN₃O₂: 396.2082, found: 396.2074.

### tert-Butyl 2-(2-fluoro-6-morpholinopyridin-3-yl)-1H-indole-1-carboxylate (Compound 64)

1,4-Dioxane (30 mL) and H₂O (6 mL) were combined and purged with N_{2(g)} for 30 min. N-Boc-indole-2-boronic acid (6) (0.24 g, 9.19×10⁻⁴ mol), compound 53 (0.25 g, 9.58×10⁻⁴ mol), Pd(dppf)Cl₂ (0.11 g, 1.50×10⁻⁴ mol, 0.16 equiv.), and Na₂CO₃ (0.30 g, 2.83 mmol, 3.1 equiv.) were flushed with N_{2(g)} for 15 min, then the H₂O/1,4-dioxane mixture (15 mL) was added. The reaction mixture was stirred at reflux under N_{2(g)} for 4 h, then cooled to ambient temperature, filtered through Celite, and the Celite was rinsed with 1,4-dioxane, then EtOAc. The solvent was removed from the filtrate to give a brown oil that was dried under vacuum to give a brown residue. The residue was dissolved in CH₂Cl₂, poured onto dry silica (55 mm h x 45 mm i.d.), and eluted under vacuum: CH₂Cl₂ (100 mL), % MeOH/CH₂Cl₂—1% (100 mL), 2.5% (100 mL), 5% (150 mL). The desired fractions were combined, concentrated, poured onto dry silica (55 mm h×45 mm i.d.), and eluted under vacuum: hexane/EtOAc/NEt₃ v/v/v 75:20:5 (300 mL), 50:45:5 (100 mL) to give a brown residue (0.23 g). Purification by radial chromatography (2 mm silica) hexane/EtOAc/NEt₃ v/v/v 90:8:2 (100 mL), 75:20:5 (100 mL) afforded Compound 64 (0.20 g, 55%) as a white foam: ¹H NMR (300 MHz, CDCl₃) δ 8.20 (d, 1H, J=8.1 Hz), 7.62 (dd, 1H, J=9.6 Hz, J=8.1 Hz), 7.54 (d, 1H, J=7.2 Hz), 7.32 (ddd, 1H, J=8.4 Hz, 1=7.2 Hz, 1=1.2 Hz), 7.23 (td, 1H, J=7.5 Hz, J=0.9 Hz), 6.55 (s, 1H), 6.48 (dd, 1H, J=8.1 Hz, J=1.8 Hz), 3.82 (t, 4H, J=4.8 Hz), 3.54 (t, 4H, J=4.8 Hz), 1.46 (s, 9H); ¹³C NMR (125 MHz, CDCl₃) δ 159.39 (d, ¹J_{FC}=237.7 Hz), 158.10 (d, J_{FC}=15.6 Hz), 150.26, 142.03 (d, J_{FC}=4.8 Hz), 137.27, 133.70 (d, J_{FC}=4.3 Hz), 129.22, 124.62, 123.01, 120.57, 115.69, 110.61, 105.72 (d, J_{FC}=31.2 Hz), 102.33 (d, J_{FC}=3.8 Hz), 83.81, 66.73, 45.64, 28.01; ¹⁹F NMR (282.5 MHz, CDCl₃) δ −67.85 (d, J=9.9 Hz); HRMS (ESI) [M+H]⁺ Calcd for C₂₂H₂₅FN₃O₃: 398.1875, found: 398.1867.

### tert-Butyl 2-(2-nitro-6-(piperidin-1-yl)pyridin-3-yl)-1H-indole-1-carboxylate (Compound 65)

1,4-Dioxane (15 mL) and H₂O (3 mL) were combined and purged with N_{2(g)} for 30 min. N-Boc-indole-2-boronic acid (6) (92 mg, 3.52×10⁻⁴ mol), compound 54 (110 mg, 3.84×10⁻⁴ mol), Pd(dppf)Cl₂ (32 mg, 4.37×10⁻⁵ mol, 0.1 equiv.), and Na₂CO₃ (196 mg, 1.85 mmol, 5.2 equiv.) were flushed with N_{2(g)} for 10 min, then the H₂O/1,4-dioxane mixture (10 mL) was added. The reaction mixture was stirred at reflux under N_{2(g)} for 5 h, then cooled to ambient temperature, filtered through Celite, and the Celite was rinsed with EtOAc. The solvent was removed from the filtrate to give a dark green/black residue that was dissolved in CH₂Cl₂, poured onto dry silica (45 mm h×45 mm i.d.), and eluted under vacuum with 75:20:5 v/v/v hexane/EtOAc/NEt₃ (200 mL) to give a dark orange syrup (0.14 g). Purification by radial chromatography (2 mm silica): hexane/EtOAc/NEt₃ v/v/v 90:8:2 (100 mL), 75:20:5 (100 mL) gave a yellow solid (65 mg) that was again purified by radial chromatography (2 mm silica) with CHCl₃ (50 mL) to give an orange foam (56 mg). Purification by radial chromatography (1 mm silica): % CH₂Cl₂/hexane—25% (25 mL), 50% (50 mL), CH₂Cl₂ (25 mL) afforded Compound 65 (48 mg, 32%) as a yellow/orange foam: ¹H NMR (400 MHz, CDCl₃) δ 8.20 (d, 1H, J=8.4 Hz), 7.58 (d, 1H, J=8.8 Hz), 7.53 (d, 1H, J=8.0 Hz), 7.33 (apparent t, 1H, J=7.6 Hz), 7.24 (apparent t—partially obscured by CHCl₃ resonance, 1H, J=7.2 Hz), 6.86 (d, 1H, J=8.8 Hz), 6.50 (s, 1H), 3.66 (m, 4H), 1.67 (m, 6H), 1.41 (s, 9H); ¹³C NMR (125 MHz, CDCl₃) δ 157.10, 154.90, 150.07, 142.88, 136.97, 134.21, 129.21, 124.83, 123.08, 120.72, 116.06, 111.29, 110.65, 109.53, 83.90, 46.24, 27.97, 25.60, 24.74; HRMS (ESI) [M+H]⁺ Calcd for C₂₃H₂₇N₄O₄: 423.2027, found: 423.2030. X-ray quality crystals were grown by slow evaporation of CH₂Cl₂/hexane.

### tert-Butyl 2-(6-morpholino-2-nitropyridin-3-yl)-1H-indole-1-carboxylate (Compound 66)

1,4-Dioxane (15 mL) and H₂O (3 mL) were combined and purged with N_{2(g)} for 30 min. N-Boc-indole-2-boronic acid (6) (60 mg, 2.30×10⁻⁴ mol), compound 55 (66 mg, 2.30×10⁻⁴ mol), Pd(dppf)Cl₂ (19 mg, 2.60×10⁻⁴ mol, 0.1 equiv.), and Na₂CO₃ (117 mg, 1.10 mmol, 4.8 equiv.) were flushed with N_{2(g)}, then the H₂O/1,4-dioxane mixture (10 mL) was added. The reaction mixture was stirred at reflux under N_{2(g)} for 4 h, then cooled to ambient temperature, filtered through Celite, and the Celite was rinsed with EtOAc. The filtrate was concentrated, then EtOAc was added and removed to give a dark green/brown oil. CH₂Cl₂ and hexane were added and removed to give a residue that was dissolved in CH₂Cl₂, poured onto dry silica (45 mm h×45 mm i.d.), and eluted under vacuum: CH₂Cl₂ (200 mL), % MeOH/CH₂Cl₂—1% (100 mL), 2% (150 mL) to give a dark yellow/brown residue (69 mg). Purification by radial chromatography (1 mm silica): CH₂Cl₂ (100 mL) afforded Compound 66 (36 mg, 37%) as a yellow/orange foam: ¹H NMR (400 MHz, CDCl₃) δ 8.18 (d, 1H, J=8.1 Hz), 7.67 (d, 1H, J=8.1 Hz), 7.54 (d, 1H, J=7.6 Hz), 7.34 (ddd, 1H, J=8.4 Hz, J=7.2 Hz, J=1.2 Hz), 7.25 (m—partially obscured by CHCl₃ resonance, 1H), 6.88 (d, 1H, J=8.4 Hz), 6.52 (s, 1H), 3.84 (t, 4H, J=4.8 Hz), 3.65 (t, 4H, J=4.8 Hz), 1.43 (s, 9H); ¹³C NMR (100 MHz, CDCl₃) δ 157.25, 154.70, 150.06, 143.25, 136.89, 133.83, 129.17, 125.00, 123.17, 120.83, 116.09, 113.03, 110.87, 109.56, 84.12, 66.67, 45.35, 28.01; HRMS (ESI) [M+H]⁺ Calcd for C₂₂H₂₅N₄O₅: 425.1820, found: 425.1815. X-ray quality crystals were grown by slow evaporation of CH₂Cl₂/hexane.

### 4-(5-(1H-Indol-2-yl)-6-nitropyridin-2-yl)morpholine (Compound 67)

Isolated as a side-product from the reaction of Compound 66: ¹H NMR (400 MHz, acetone-d₆) δ 10.53 (br s, 1H), 8.09 (d, 1H, J=8.8 Hz), 7.56 (d, 1H, J=7.6 Hz), 7.41 (d, 1H, J=8.4 Hz), 7.20 (d, 1H, J=8.8 Hz), 7.13 (t, 1H, J=7.6 Hz), 7.04 (t, 1H, J=7.6 Hz), 6.49 (s, 1H), 3.77 (t, 4H, J=4.8 Hz), 3.62 (t, 4H, J=4.8 Hz); ¹³C NMR (125 MHz, DMSO-d₆) δ 156.31, 154.79, 140.95, 136.77, 130.23, 128.21, 121.94, 120.14, 119.51, 111.30, 110.32, 107.21, 100.09, 65.66, 44.65; HRMS (ESI) [M+H]⁺ Calcd for C₁₇H₁₇N₄O₃: 325.1295, found: 325.1295.

### 2-(2-Fluoro-6-(piperidin-1-yl)pyridin-3-yl)-1H-indole (Compound 68)

Compound 63 (71 mg, 1.80×10⁻⁴ mol) and TFA (1.8 mL, 23.4 mmol, 130 equiv.) were stirred for 20 min, then poured into a mixture of NaHCO₃ (2.29 g, 27.26 mmol, 1.2 equiv. TFA), H₂O (45 mL), and CH₂Cl₂ (45 mL). The mixture was stirred for 10 min, the layers were separated, and the aqueous layer was extracted with CH₂Cl₂ (20 mL). The combined CH₂Cl₂ layers were washed with brine (25 mL), dried over MgSO₄, concentrated, poured onto dry silica (33 mm h×33 mm i.d.), and eluted under vacuum: hexane/EtOAc/NEt₃ v/v/v 90:8:2 (25 mL), 75:20:5 (200 mL), 50:45:5 (50 mL) to afford Compound 68 (25 mg, 47%) as an off-white solid: ¹H NMR (400 MHz, CDCl₃) δ 8.77 (br s, 1H), 7.92 (dd, 1H, J=10.6 Hz, J=8.4 Hz), 7.59 (d, 1H, J=8.0 Hz), 7.39 (d, 1H, J=7.6 Hz), 7.17 (td, 1H, J=7.6 Hz, J=0.8 Hz), 7.10 (td, 1H, J=7.6 Hz, J=0.8 Hz), 6.72 (d, 1H, J=1.6 Hz), 6.54 (dd, 1H, J=8.4 Hz, J=2.4 Hz), 3.59 (m, 4H), 1.67 (m, 6H); ¹³C NMR (125 MHz, DMSO-d₆) δ 158.00 (d, ¹J_{FC}=236.7 Hz), 156.04 (d, J_{FC}=16.5 Hz), 139.16 (d, J_{FC}=4.4 Hz), 136.42, 131.47 (d, J_{FC}=7.3 Hz), 128.57, 121.11, 119.60, 119.21, 111.00, 103.91 (d, J_{FC}=3.5 Hz), 100.65 (d, J_{FC}=28.0 Hz), 99.81 (d, J_{FC}=8.3 Hz), 45.44, 24.93, 24.09; ¹⁹F NMR (470.6 MHz, DMSO-d₆) δ −67.52; ¹⁹F NMR (376.5 MHz, CDCl₃) δ −70.32; HRMS (ESI) [M+H]⁺ Calcd for C₁₈H₁₉FN₃: 296.1558, found: 296.1604.

### 4-(6-Fluoro-5-(1H-indol-2-yl)pyridin-2-yl)morpholine (Compound 69, JSS20-183A)

Compound 64 (76 mg, 1.91×10⁻⁴ mol) and TFA (1.8 mL, 23.4 mmol, 123 equiv.) were stirred for 20 min, then poured into a mixture of NaHCO₃ (2.22 g, 26.43 mmol, 1.1 equiv. TFA), H₂O (45 mL), and CH₂Cl₂ (45 mL). The mixture was stirred for 10 min, the layers were separated, and the aqueous layer was extracted with CH₂Cl₂ (20 mL). The combined CH₂Cl₂ layers were washed with brine (25 mL), dried over MgSO₄, concentrated, poured onto dry silica (33 mm h x 33 mm i.d.), and eluted under vacuum: hexane/EtOAc/NEt₃ v/v/v 75:20:5 (100 mL), 50:45:5 (200 mL), 20:75:5 (75 mL) to afford Compound 69 (54 mg, 95%) as a light tan solid: ¹H NMR (400 MHz, CDCl₃) δ 8.78 (br s, 1H), 7.99 (dd, 1H, J=10.4 Hz, J=8.8 Hz), 7.60 (d, 1H, J=7.6 Hz), 7.40 (d, 1H, J=8.0 Hz), 7.18 (td, 1H, J=7.6 Hz, J=0.8 Hz), 7.11 (td, 1H, J=7.6 Hz, J=0.8 Hz), 6.77 (d, 1H, J=1.2 Hz), 6.55 (dd, 1H, J=8.4 Hz, J=2.0 Hz), 3.82 (t, 4H, J=4.8 Hz), 3.57 (t, 4H, J=4.8 Hz); ¹³C NMR (125 MHz, DMSO-d₆) δ 157.86 (d, ¹J_{FC}=237.2 Hz), 156.31 (d, J_{FC}=16.1 Hz), 139.26 (d, J_{FC}=4.4 Hz), 136.47, 131.13 (d, J_{FC}=7.2 Hz), 128.50, 121.30, 119.71, 119.27, 111.06, 104.14 (d, J_{FC}=3.6 Hz), 102.11 (d, J_{FC}=27.5 Hz), 100.23 (d, J_{FC}=8.6 Hz), 65.71, 44.83; ¹⁹F NMR (376.5 MHz, CDCl₃) δ −70.37; HRMS (ESI) [M+H]+Calcd for C₁₇H₁₇FN₃O: 298.1350, found: 298.1396. X-ray quality crystals were grown by slow evaporation of CDCl₃.

### tert-Butyl 5-fluoro-2-(2-morpholinopyrimidin-5-yl)-1H-indole-1-carboxylate (Compound 70)

Compound 42 (0.21 g, 7.52×10⁻⁴ mol), compound 24 (0.10 g, 4.10×10⁻⁴ mol), and Pd(dppf)Cl₂ (20 mg, 2.73×10⁻⁵ mol) were dissolved in 1,4-dioxane (10 mL), then K₂CO_{3(aq)} (2 M, 0.5 mL) was added. The reaction mixture was flushed with N_{2(g)} for 10 min, then heated at 100° C. under N_{2(g)} overnight. The reaction mixture was cooled to ambient temperature, evaporated to dryness, the residue was dissolved in EtOAc (30 ml), washed with H₂O (10 mL), dried over MgSO₄, and filtered. The filtrate was passed through a silica gel pad, the silica was washed with EtOAc, and the filtrate was concentrated to dryness. The residue was purified by radial chromatography (hexane/EtOAc v/v 90:10 to 80:20) to afford Compound 70 (0.15 g, 92%) as a solid: ¹H NMR (300 MHz, CDCl₃) δ 8.38 (s, 2H), 8.13 (dd, 1H, J=9.0 Hz, J=4.5 Hz), 7.20 (dd, 1H, J=8.7 Hz, J=2.7 Hz), 7.05 (td, 1H, J=9.0 Hz, J=2.7 Hz), 6.50 (d, 1H, J=0.3 Hz), 3.88 (m, 4H), 3.79 (m, 4H), 1.49 (s, 9H); HRMS (ESI) [M+H]⁺ Calcd for C₂₁H₂₄O₃N₄F: 399.1827, found: 399.1837.

### tert-Butyl 5-fluoro-2-(6-morpholinopyridin-3-yl)-1H-indole-1-carboxylate (Compound 71)

Compound 42 (0.21 g, 7.52×10⁻⁴ mol), compound 29 (0.10 g, 4.11×10⁻⁴ mol), Pd(dppf)Cl₂ (20 mg, 2.73×10⁻⁵ mol), 1,4-dioxane (10 mL), and K₂CO_{3(aq)} (2 M, 0.5 mL) were flushed with N_{2(g)} for 10 min, then stirred at 100° C. under N_{2(g)} overnight. The reaction mixture was cooled to ambient temperature, evaporated to dryness, the residue was dissolved in EtOAc (30 ml), washed with H₂O (10 mL), dried over MgSO₄, and filtered. The filtrate was passed through a silica gel pad, the silica was washed with EtOAc, and the filtrate was concentrated to dryness. The residue was purified by radial chromatography (hexane/EtOAc v/v 90:10 to 80:20) to afford Compound 71 (0.12 g, 73%) as a solid: ¹H NMR (300 MHz, CDCl₃) δ 8.26 (d, 1H, J=2.1 Hz), 8.12 (dd, 1H, J=9.0 Hz, J=4.5 Hz), 7.55 (dd, 1H, J=8.7 Hz, J=1.8 Hz), 7.19 (dd, 1H, J=8.7 Hz, J=2.4 Hz), 7.03 (td, 1H, J=9.0 Hz, 0.1=2.4 Hz), 6.68 (d, 1H, J=8.7 Hz), 6.48 (s, 1H), 3.85 (t, 4H, J=4.8 Hz), 3.57 (partially resolved t, 4H, J=4.8 Hz), 1.43 (s, 9H); HRMS (ESI) [M+H]⁺ Calcd for C₂₂H₂₅O₃N₃F: 398.1875, found: 398.1887.

### 4-(5-(5-Fluoro-1H-indol-2-yl)pyrimidin-2-yl)morpholine (Compound 72)

Compound 70 (0.14 g, 3.51×10⁻⁴ mol) was dissolved in TFA (2 mL) and stirred at ambient temperature for 2 h, then the TFA was removed with N_{2(g)} flow. The residue was neutralized with aqueous NaHCO₃ to give a solid that was washed with cold CH₂Cl₂ to afford Compound 72 (58 mg, 55%) as an off-white solid: ¹H NMR (300 MHz, acetone-d₆) δ 10.80 (br s, 1H), 8.83 (s, 2H), 7.37 (dd, 1H, J=8.7 Hz, J=4.5 Hz), 7.23 (dd, 1H, J=9.6 Hz, J=2.4 Hz), 6.88 (dd, 1H, J=9.6 Hz, J=8.7 Hz, J=2.4 Hz), 6.79 (m, 1H), 3.82 (m, 4H), 3.71 (m, 4H); ¹³C NMR (125 MHz, DMSO-d₆): 160.33, 157.23 (d, ¹J_{FC}=231.4 Hz), 154.69, 135.15, 133.59, 128.93 (d, J_{FC}=10.3 Hz), 115.41, 111.92 (d, J_{FC}=9.9 Hz), 109.27 (d, J_{FC}=25.9 Hz), 104.31, 104.12, 97.50 (d, J_{FC}=4.7 Hz), 65.95, 44.01; HRMS (ESI) [M+H]⁺ Calcd for C₁₆H₁₆ON₄F: 299.1303, found: 299.1301.

### 4-(5-(5-fluoro-1H-indol-2-yl)pyridin-2-yl)morpholine (Compound 73)

Compound 71 (0.12 g, 3.02×10⁻⁴ mol) was dissolved in TFA (2 mL) and stirred at ambient temperature for 2 h, then the TFA was removed with N_{2(g)} flow. The residue was neutralized with aqueous NaHCO₃ to give a solid that was placed in a filter and rinsed with a small amount of acetone to afford Compound 73 (66 mg, 74%) as an off-white solid: ¹H NMR (acetone-d₆, 300 MHz): δ 10.62 (s, 1H), 8.65 (dd, 1H, J=2.5 Hz, J=0.7 Hz), 7.98 (dd, 1H, J=8.9 Hz, J=2.6 Hz), 7.34 (dd, 1H, J=8.8 Hz, J=4.5 Hz), 7.21 (dd, 1H, J=9.9 Hz, J=2.5 Hz), 6.85 (m, 2H), 6.74 (s, 1H), 3.74 (m, 4H), 3.56 (m, 4H); ³C NMR (125 MHz, DMSO-d₆): 158.30, 157.17 (d, ¹J_{FC}=231.5 Hz), 144.57, 137.81, 134.38, 133.54, 129.07 (d, J_{FC}=10.6 Hz), 117.78, 111.75 (d, J_{FC}=9.9 Hz), 108.88 (d, J_{FC}=26.0 Hz), 106.86, 104.05 (d, J_{FC}=23.1 Hz), 97.18 (d, J_{FC}=4.5 Hz), 65.90, 45.01; HRMS (ESI) [M+H]⁺ Calcd for C₁₇H₁₇ON₃F: 298.1350, found: 298.1348.

### tert-Butyl 6-fluoro-2-(2-fluoro-6-(piperidin-1-yl)pyridin-3-yl)-1H-indole-1-carboxylate (Compound 74)

1,4-Dioxane (25 mL) was purged with N_{2(g)} for 1 h. Compound 52 (0.25 g, 9.65×10⁻⁴ mol), compound 4 (0.41 g, 1.03 mmol, 1.1 equiv.), Na₂CO₃ (0.51 g, 4.81 mmol, 5 equiv.), and Pd(dppf)Cl₂ (89 mg, 1.22×10⁻⁴ mol, 0.1 equiv.) were flushed with N_{2(g)} for 10 min, then the 1,4-dioxane was added. The reaction mixture was stirred at reflux under N_{2(g)} for 3 h, then cooled to ambient temperature, filtered through Celite, and the Celite was rinsed with EtOAc. The filtrate was concentrated to a brown oil, then hexane was added and removed to give a brown residue that was dissolved in CH₂Cl₂, poured onto dry silica (55 mm h×45 mm i.d.), and eluted under vacuum: hexane (50 mL), hexane/EtOAc/NEt₃ v/v/v 90:8:2 (100 mL), 75:20:5 (100 mL), 50:45:5 (50 mL) to give an orange syrup (0.43 g). Purification by radial chromatography (2 mm silica): hexane/EtOAc/NEt₃ v/v/v 95:4:1 (100 mL), 90:8:2 (50 mL) gave an off-white foam (0.16 g) that was again purified by radial chromatography (2 mm silica): % CH₂Cl₂/hexane—25% (50 mL), 50% (75 mL), 75% (50 mL), CH₂Cl₂ (25 mL) to give an off-white solid (0.14 g). A final purification by radial chromatography (2 mm silica): hexane/EtOAc/NEt₃ v/v/v 90:8:2 (125 mL), 75:20:5 (25 mL) afforded Compound 74 (0.13 g, 33%) as a white solid: ¹H NMR (300 MHz, CDCl₃) δ 7.95 (dd, 1H, J=10.8 Hz, J=2.1 Hz), 7.54 (dd, 1H, J=9.6 Hz, J=8.4 Hz), 7.44 (dd, 1H, J=8.4 Hz, J=5.4 Hz), 6.99 (td, 1H, J=8.7 Hz, J=2.4 Hz), 6.54 (d, 1H, J=7.5 Hz), 6.49 (s, 1H), 3.57 (m, 4H), 1.67 (s, 6H), 1.44 (s, 9H); ¹³C NMR (100 MHz, CDCl₃) δ 161.06 (d, J_{FC}=238.0 Hz), 159.46 (d, J_{FC}=234.5 Hz), 158.04 (d, J_{FC}=16.2 Hz), 150.10, 141.80 (d, J_{FC}=4.8 Hz), 137.51 (d, J_{FC}=12.8 Hz), 134.52 (t, J_{FC}=4.3 Hz), 125.55 (d, J_{FC}=1.1 Hz), 120.95 (d, J_{FC}=9.8 Hz), 111.16 (d, J_{FC}=24.1 Hz), 109.84, 103.46 (d, J_{FC}=31.3 Hz), 103.11 (d, J_{FC}=28.6 Hz), 102.25 (d, J_{FC}=3.8 Hz), 84.08, 46.45, 27.93, 25.55, 24.84; HRMS (ESI) [M+H]⁺ Calcd for C₂₃H₂₆F₂N₃O₂: 414.1988, found: 414.1989.

### 6-Fluoro-2-(2-fluoro-6-(piperidin-1-yl)pyridin-3-yl)-1H-indole (Compound 75)

Compound 74 (92 mg, 2.23×10⁻⁴ mol) was dissolved in TFA (2.2 mL, 28.6 mmol, 128 equiv.), stirred at ambient temperature for 20 min, then poured into a mixture of NaHCO₃ (2.68 g, 31.90 mmol, 1.1 equiv. TFA), H₂O (55 mL), and CH₂Cl₂ (55 mL). The mixture was stirred for 10 min, then the layers were separated, and the H₂O layer was extracted with CH₂Cl₂ (25 mL). The combined CH₂Cl₂ layers were washed with H₂O (50 mL), brine (50 mL), and dried over MgSO₄. The solution was concentrated, poured onto dry silica (55 mm h×45 mm i.d.), and eluted under vacuum: hexane (50 mL), hexane/EtOAc/NEt₃ v/v/v 75:20:5 (200 mL), 50:45:5 (150 mL) to give an off-white/tan solid (68 mg). The solid was suspended in CHCl₃ (1 mL), filtered, rinsed with CHCl₃ (1 mL×3), then hexane (5 mL), and dried under vacuum to afford Compound 75 (49 mg, 70%) as a white solid: ¹H NMR (300 MHz, DMSO-d₆) δ 11.36 (s, 1H), 8.08 (dd, 1H, J=10.8 Hz, J=8.7 Hz), 7.49 (dd, 1H, J=8.7 Hz, J=5.7 Hz), 7.13 (dd, 1H, J=9.9 Hz, J=1.8 Hz), 6.84 (m, 2H), 6.69 (s, 1H), 3.56 (m, 4H), 1.59 (m, 6H); ¹³C NMR (125 MHz, DMSO-d₆) δ 158.71 (d, ¹J_{FC}=232.9 Hz), 157.84 (d, ¹J_{FC}=234.8 Hz), 157.05 (d, J_{FC}=16.3 Hz), 139.06 (d, J_{FC}=4.4 Hz), 136.33 (d, J_{FC}=12.9 Hz), 132.23 (dd, J_{FC}=7.3 Hz, J_{FC}=3.6 Hz), 125.34, 120.53 (d, J_{FC}=10.0 Hz), 107.61 (d, J_{FC}=24.3 Hz), 103.91 (d, J_{FC}=3.5 Hz), 100.40 (d, J_{FC}=27.9 Hz), 99.70 (d, J_{FC}=8.0 Hz), 97.03 (d, J_{FC}=25.8 Hz), 45.41, 24.92, 24.06; HRMS (ESI) [M+H]Calcd for C₁₈H₁₈F₂N₃: 314.1463, found: 314.1454.

### (1-(tert-Butoxycarbonyl)-6-fluoro-1H-indol-2-yl)boronic acid (Compound 76)

Compound 104 (2.35 g, 9.99 mmol) was dissolved in THE (100 mL) under N_{2(g)}, cooled to 0° C., then triisopropyl borate (2.82 g, 14.99 mmol) was added. LDA (2 M THF/heptane/ethylbenzene, 6.5 mL, 13 mmol) was added dropwise over a period of 10 min, the reaction mixture was stirred at 0° C. for 2 h, then quenched with 1 M HCl_{(aq)}. The reaction mixture was extracted with EtOAc (100 mL×3), the combined extracts were washed with H₂O (2×50 ml), dried over MgSO₄, filtered, and evaporated to dryness. The residue was recrystallized from 1:2 v/v hexane/EtOAc to afford Compound 76 (1.78 g, 64%) as a white solid: ¹H NMR (300 MHz, CDCl₃): δ 7.72 (dd, 1H, J=11.1 Hz, J=2.4 Hz), 7.52 (dd, 1H, J=8.4 Hz, J=5.7 Hz), 7.45 (d, 1H, J=0.6 Hz), 7.02 (td, 1H, J=8.7 Hz, J=2.4 Hz) 6.88 (br s, 2H), 1.75 (s, 9H).

### tert-Butyl 6-fluoro-2-(5-morpholinopyrazin-2-yl)-1H-indole-1-carboxylate (Compound 77)

Compound 76 (0.21 g, 7.52×10⁻⁴ mol), compound 30 (0.10 g, 4.10×10⁻⁴ mol), Pd(dppf)Cl₂ (20 mg, 2.73×10⁻⁵ mol), 1,4-dioxane (10 mL), and K₂CO_{3(aq)} (2 M, 0.5 mL) were flushed with N_{2(g)} for 10 min, then stirred under N_{2(g)} at 100° C. overnight. The reaction mixture was cooled to ambient temperature, evaporated to dryness, the residue was dissolved in EtOAc (30 ml), washed with H₂O (10 mL), dried over MgSO₄, and filtered. The filtrate was passed through a silica gel pad, the silica was washed with EtOAc, and the filtrate was concentrated to dryness. The residue was purified by radial chromatography (hexane/EtOAc v/v 90:10 to 80:20) to afford Compound 77 (78 mg, 48%) as a solid: ¹H NMR (300 MHz, CDCl₃) δ 8.27 (d, 1H, J=1.2 Hz), 8.17 (d, 1H, J=1.5 Hz), 7.88 (dd, 1H, J=10.8 Hz, J=2.4 Hz), 7.48 (dd, 1H, J=8.7 Hz, J=5.7 Hz), 7.01 (td, 1H, J=9.0 Hz, J=2.4 Hz), 6.69 (s, 1H), 3.86 (apparent t, 4H, J=4.8 Hz), 3.62 (apparent t, 4H, J=4.8 Hz), 1.44 (s, 9H); HRMS (ESI) [M+H]⁺ Calcd for C₂₁H₂₄O₃N₄F: 399.1827, found: 399.1829.

### tert-Butyl 6-fluoro-2-(4-fluoro-6-morpholinopyridin-3-yl)-1H-indole-1-carboxylate (Compound 78)

Compound 76 (0.34 g, 1.22 mmol), compound 57 (0.15 g, 5.75×10⁻⁴ mol), Pd(dppf)Cl₂ (28 mg, 3.83×10⁻⁵ mol), 1,4-dioxane (30 mL), and K₂CO_{3(aq)} (2 M, 0.7 mL) were flushed with N_{2(g)} for 5 min, then stirred under N_{2(g)} at 100° C. overnight (TLC indicated unreacted Compound 57 remaining). Additional Compound 76 (0.15 g, 5.37×10⁻⁴ mol) was added and the reaction mixture was stirred under N_{2(g)} at 100° C. overnight, then cooled to ambient temperature and evaporated to dryness. The residue was dissolved in H₂O, extracted with EtOAc (10 ml×3), the combined extracts were dried over MgSO₄, and filtered. The filtrate was passed through a silica gel pad, the silica was washed with EtOAc, and the solvent was removed to give a residue that was purified by radial chromatography (hexane/EtOAc v/v 99:1 to 80:20) to afford Compound 78 (0.15 g, 63%) as a solid: ¹H NMR (500 MHz, CDCl₃) δ 8.20 (d, 1H, J=10.5 Hz), 7.92 (dd, 1H, J=10.5 Hz, J=1.5 Hz), 7.47 (dd, 1H, J=9.0 Hz, J=5.5 Hz), 7.01 (td, 1H, J=9.0 Hz, J=2.5 Hz), 6.53 (s, 1H), 6.36 (d, 1H, J=12.5 Hz), 3.83 (t, 4H, J=5.0 Hz), 3.56 (partially resolved m, 4H), 1.47 (s, 9H); HRMS (ESI) [M+H]⁺ Calcd for C₂₂H₂₄O₃N₃F₂: 416.1780, found: 416.1793.

### tert-Butyl 6-fluoro-2-(5-fluoro-6-(piperidin-1-yl)pyridin-3-yl)-1H-indole-1-carboxylate (Compound 79)

Compound 76 (60 mg, 2.15×10⁻⁴ mol), compound 58 (53 mg, 2.05×10⁻⁴ mol), Pd(dppf)Cl₂ (10 mg, 1.37×10⁻⁵ mol), 1,4-dioxane (6 mL), and K₂CO_{3(aq)} (2 M, 0.3 mL) were flushed with N_{2(g)} for 5 min, then stirred under N_{2(g)} at 100° C. overnight (TLC indicated unreacted Compound 58 remaining). Additional Compound 76 (64 mg, 2.29×10⁻⁴ mol) was added and the reaction mixture was stirred under N_{2(g)} at 100° C. overnight, then cooled to ambient temperature and evaporated to dryness. The residue was dissolved in H₂O, extracted with EtOAc (10 ml×3), the combined extracts were dried over MgSO₄, and filtered. The filtrate was passed through a silica gel pad, the silica was washed with EtOAc, and the solvent was removed to give a residue that was purified by radial chromatography (hexane/EtOAc v/v 99:1 to 80:20) to afford Compound 79 (59 mg, 70%) as a solid: ¹H NMR (300 MHz, CDCl₃) δ 8.00 (m, 1H), 7.89 (dd, 1H, J=10.8 Hz, J=1.8 Hz), 7.41 (m, 1H), 7.21 (dd, 1H, J=13.8 Hz, J=1.8 Hz), 6.96 (tt, 1H, J=9.0 Hz, J=2.1 Hz), 6.47 (s, 1H), 3.45 (m, 4H), 1.64 (m, 6H), 1.39 (s, 9H); HRMS (ESI) [M+H]⁺ Calcd for C₂₃H₂₆O₂N₃F₂: 414.1988, found: 414.1988.

### tert-Butyl 6-fluoro-2-(5-fluoro-6-morpholinopyridin-3-yl)-1H-indole-1-carboxylate (Compound 80)

Compound 76 (0.19 g, 6.81×10⁻⁴ mol), compound 59 (77 mg, 2.95×10⁻⁴ mol), Pd(dppf)Cl₂ (12 mg, 1.64×10⁻⁵ mol), 1,4-dioxane (10 mL), and K₂CO_{3(aq)} (2 M, 0.5 mL) were flushed with N_{2(g)} for 10 min, then stirred under N_{2(g)} at 100° C. overnight. The reaction mixture was cooled to ambient temperature, evaporated to dryness, the residue was dissolved in H₂O, and extracted with EtOAc (10 ml×3). The combined extracts were dried over MgSO₄ and filtered, the filtrate was passed through a silica gel pad, and the silica was washed with EtOAc. The solvent was removed to give a residue that was purified by radial chromatography (hexane/EtOAc v/v 95:5 to 80:20) to afford Compound 80 (86 mg, 70%) as a solid: ¹H NMR (400 MHz, CDCl₃): δ 8.07 (s, 1H), 7.92 (d, 1H, J=10.8 Hz), 7.46 (dd, 1H, J=8.8 Hz, J=5.6 Hz), 7.30 (d, 1H, J=13.6 Hz), 7.02 (unresolved apparent td, 1H, J=8.8 Hz), 6.53 (s, 1H), 3.86 (t, 4H, J=4.4 Hz), 3.54 (t, 4H, J=4.4 Hz), 1.45 (s, 9H), HRMS (ESI) [M+H]r Calcd for C₂₂H₂₄O₃N₃F₂: 416.1780, found: 416.1781.

### 4-(5-(6-Fluoro-1H-indol-2-yl)pyrazin-2-yl)morpholine (Compound 81)

Compound 77 (78 mg, 1.96×10⁻⁴ mol) was dissolved in TFA (2 mL), stirred at ambient temperature for 2 h, then the TFA was removed with N_{2(g)} flow. The residue was neutralized with aqueous NaHCO₃ to give a solid that was washed with cold CH₂Cl₂ to afford Compound 81 (58 mg, 99%) as an off-white solid: ¹H NMR (500 MHz, acetone-d₆) δ 10.75 (br s, 1H), 8.72 (d, 1H, J=1.0 Hz), 8.26 (d, 1H, J=1.5 Hz), 7.55 (dd, 1H, J=9.0 Hz, J=5.5 Hz), 7.23 (dd, 1H, J=10.0 Hz, J=2.0 Hz), 6.84 (ddd, 1H, J=10.0 Hz, J=8.5 Hz, J=2.0 Hz), 3.79 (t, 4H, J=5.0 Hz), 3.62 (t, 4H, J=5.0 Hz), ¹³C NMR (125 MHz, DMSO-d₆): 159.06 (d, ¹J_{FC}=234.3 Hz), 153.41, 138.40, 136.93 (d, J_{FC}=13.2 Hz), 136.29, 135.17, 129.82, 125.49, 121.08 (d, J_{FC}=9.6 Hz), 107.89 (d, J_{FC}=24.9 Hz), 98.09, 97.51 (d, J_{FC}=26.0 Hz), 65.81, 44.54; HRMS (ESI) [M+H]⁺ Calcd for C₁₆H₁₆ON₄F: 299.1303, found: 299.1302.

### 4-(4-Fluoro-5-(6-fluoro-1H-indol-2-yl)pyridin-2-yl)morpholine (Compound 82)

Compound 78 (0.15 g, 3.61×10⁻⁴ mol) was dissolved in TFA (2 mL), stirred at ambient temperature for 1.5 h, then the TFA was removed with N_{2(g)} flow. The residue was neutralized with aqueous NaHCO₃ to give a solid that was washed with cold CH₂Cl₂ to afford Compound 82 (0.11 g, 97%) as an off-white solid: ¹H NMR (300 MHz, CDCl₃) δ 8.71 (br s, 1H), 8.61 (d, 1H, J=11.1 Hz), 7.52 (dd, 1H, J=8.7 Hz, J=5.4 Hz), 7.08 (dd, 1H, J=9.6 Hz, J=2.1 Hz), 6.89 (ddd, 1H, J=9.9 Hz, J=8.7 Hz, J=2.1 Hz), 6.80 (dd, 1H, J=1.2 Hz), 6.40 (d, 1H, J=14.7 Hz), 3.85 (apparent t, 4H, J=4.8 Hz), 3.57 (apparent t, 4H, J=4.8 Hz); ¹³C NMR (125 MHz, DMSO-d₆): 166.26 (d, ¹J_{FC}=257.9 Hz), 159.88 (d, J_{FC}=4.5 Hz), 158.99 (d, J_{FC}=241.6 Hz), 147.42 (d, J_{FC}=4.9 Hz), 136.47 (d, J_{FC}=12.7 Hz), 130.55, 125.23, 120.82 (d, J_{FC}=10.1 Hz), 107.83 (d, J_{FC}=24.4 Hz), 107.36 (d, J_{FC}=10.9 Hz), 100.68 (d, J_{FC}=7.0 Hz), 97.18 (d, J_{FC}=25.7 Hz), 93.74 (d, J_{FC}=22.6 Hz), 65.83, 45.03; HRMS (ESI) [M+H]⁺ Calcd for C₁₇H₁₆ON₃F₂: 316.1256, found: 316.1246.

### 6-Fluoro-2-(5-fluoro-6-(piperidin-1-yl)pyridin-3-yl)-1H-indole (Compound 83)

Compound 79 (59 mg, 1.43×10⁻⁴ mol) was dissolved in TFA (1 mL), stirred at ambient temperature for 2.5 h, then the TFA was removed with N_{2(g)} flow. The residue was neutralized with aqueous NaHCO₃ to give a solid that was purified by radial chromatography (hexanes/EtOAc v/v 95:5 to 80:20) to afford Compound 83 (30 mg, 67%) as an off-white solid: ¹H NMR (300 MHz, acetone-d₆) δ 10.74 (br s, 1H), 8.47 (t, 1H, J=1.8 Hz), 7.78 (dd, 1H, J=14.7 Hz, J=2.1 Hz), 7.53 (dd, 1H, J=8.7 Hz, J=5.4 Hz), 7.10 (dd, 1H, J=9.9 Hz, J=2.4 Hz), 6.86 (m, 1H), 6.84 (ddd, 1H, J=9.9 Hz, J=8.4 Hz, J=2.4 Hz), 3.51 (m, 4H), 1.67 (m, 6H); ¹³C NMR (125 MHz, DMSO-d₆): 159.12 (d, J_{FC}=235.1 Hz), 149.17 (d, J_{FC}=255.3 Hz), 148.54 (d, J_{FC}=6.4 Hz), 139.22 (d, J_{FC}=4.5 Hz), 137.06 (d, J_{FC}=12.7 Hz), 135.17, 125.53, 120.91 (d, J_{FC}=10.1 Hz), 120.73, 119.83 (d, J_{FC}=20.8 Hz), 107.98 (d, J_{FC}=24.4 Hz), 98.56, 97.22 (d, J_{FC}=25.5 Hz), 48.37 (d, J_{FC}=5.7 Hz), 25.49, 24.31; HRMS (ESI) [M+H]⁺ Calcd for C₁₈H₁₈N₃F₂: 314.1463, found: 314.1455.

### 4-(3-Fluoro-5-(6-fluoro-1H-indol-2-yl)pyridin-2-yl)morpholine (Compound 84)

Compound 80 (80 mg, 1.93×10⁻⁴ mol) was dissolved in TFA (2 mL), stirred at ambient temperature for 1.5h, then the TFA was removed with N_{2(g)} flow. The residue was neutralized with aqueous NaHCO₃ to give a solid that was washed with cold CH₂Cl₂ to afford Compound 84 (60 mg, 98%) as an off-white solid: ¹H NMR (300 MHz, acetone-d₆) δ 10.79 (br s, 1H), 8.51 (apparent t, 1H, J=1.8 Hz), 7.84 (dd, 1H, J=14.4 Hz, J=1.8 Hz), 7.54 (dd, 1H, J=8.7 Hz, J=5.4 Hz), 7.11 (apparent dd, 1H, J=9.9 Hz, J=2.4 Hz), 6.89 (m, 1H), 6.85 (ddd, 1H, J=9.9 Hz, J=8.7 Hz, J=2.4 Hz), 3.78 (apparent t, 4H, J=4.8 Hz), 3.50 (apparent t, 4H, J=4.8 Hz); ¹³C NMR (125 MHz, DMSO-d₆): 159.01 (d, J_{FC}=235.2 Hz), 149.21 (d, J_{FC}=255.4 Hz), 147.86 (d, J_{FC}=6.8 Hz), 139.13 (d, J_{FC}=4.7 Hz), 136.94 (d, J_{FC}=12.7 Hz), 134.79 (d, J_{FC}=3.2 Hz), 125.33, 121.48 (d, J_{FC}=3.0 Hz), 120.90 (d, J_{FC}=10.1 Hz), 119.98 (d, J_{FC}=20.6 Hz), 107.98 (d, J_{FC}=24.4 Hz), 98.82, 97.17 (d, J_{FC}=25.5 Hz), 65.97, 47.67 (d, J_{FC}=5.4 Hz); HRMS (ESI) [M+H]⁺ Calcd for C₁₇H₁₆ON₃F₂: 316.1256, found: 316.1253.

### tert-Butyl 2-(4-fluoro-6-morpholinopyridin-3-yl)-1H-indole-1-carboxylate (Compound 85)

Compound 6 (0.30 g, 1.15 mmol), compound 57 (0.15 g, 5.75×10⁻⁴ mol), Pd(dppf)Cl₂ (28 mg, 3.83×10⁻⁵ mol), 1,4-dioxane (30 mL), and K₂CO_{3(aq)} (2 M, 0.7 mL) were flushed with N_{2(g)} for 10 min, stirred under N_{2(g)} at 100° C. overnight, then cooled to ambient temperature and evaporated to dryness. The residue was dissolved in H₂O, extracted with EtOAc (10 ml×3), the extracts were combined, dried over MgSO₄, and filtered. The filtrate was passed through a silica gel pad, and the silica was washed with EtOAc. The solvent was removed to give a residue that was purified by radial chromatography (hexane/EtOAc v/v 95:5 to 80:20) to afford Compound 85 (0.16 g, 70%) as a solid: ¹H NMR (300 MHz, CDCl₃) δ 8.22 (d, 1H, J=10.5 Hz), 8.19 (d, 1H, J=7.5 Hz), 7.56 (dm, 1H, J=7.5 Hz), 7.33 (ddd, 1H, J=8.4 Hz, J=7.2 Hz, J=1.5 Hz), 7.24 (td, 1H, J=7.5 Hz, J=1.2 Hz), 6.57 (s, 1H), 6.36 (d, 1H, J=12.9 Hz), 3.83 (apparent t, 4H, J=4.8 Hz), 3.56 (apparent t, 4H, J=4.8 Hz), 1.48 (s, 9H); HRMS (ESI) [M+H]⁺ Calcd for C₂₂H₂₅O₃N₃F: 398.1875, found: 398.1887.

### tert-Butyl 2-(5-fluoro-6-(piperidin-1-yl)pyridin-3-yl)-1H-indole-1-carboxylate (Compound 86)

Compound 6 (90 mg, 3.45×10⁻⁴ mol), compound 58 (90 mg, 3.47×10⁻⁴ mol), Pd(dppf)Cl₂ (19 mg, 2.60×10⁻⁵ mol), 1,4-dioxane (10 mL), and K₂CO_{3(aq)} (2M, 0.5 mL) were flushed with N_{2(g)} for 5 min, then stirred under N_{2(g)} at 100° C. overnight (TLC indicated unreacted Compound 58 remaining). Additional Compound 6 (85 mg, 3.26×10⁻⁴ mol) was added, the reaction mixture was stirred under N_{2(g)} at 100° C. for 5 h, then cooled to ambient temperature and evaporated to dryness. The residue was dissolved in H₂O, extracted with EtOAc (10 ml×3), the extracts were combined, dried over MgSO₄, and filtered. The filtrate was passed through a silica gel pad, and the silica was washed with EtOAc. The solvent was removed to give a residue that was purified by radial chromatography (hexane/EtOAc v/v 95:5 to 80:20) to afford Compound 86 (0.12 g, 87%) as a solid: ¹H NMR (300 MHz, CDCl₃) δ 8.19 (d, 1H, J=8.4 Hz), 8.07 (t, 1H, J=1.5 Hz), 7.54 (unresolved apparent dd, 1H, J=7.8 Hz), 7.33 (partially resolved ddd, 1H, J=8.1 Hz, J=7.2 Hz, J=1.2 Hz), 7.26 (m—obscured by CHCl₃ resonance, 3H) 6.55 (s, 1H), 3.50 (m, 4H), 1.69 (m, 6H), 1.44 (s, 9H); HRMS (ESI) [M+H]⁺ Calcd for C₂₃H₂₇O₂N₃F: 396.2082, found: 396.2082.

### tert-Butyl 2-(5-fluoro-6-morpholinopyridin-3-yl)-1H-indole-1-carboxylate (Compound 87)

Compound 6 (0.17 g, 6.51×10⁻⁴ mol), compound 59 (75 mg, 2.87×10⁻⁴ mol), Pd(dppf)Cl₂ (11 mg, 1.50×10⁻⁵ mol), 1,4-dioxane (10 mL), and K₂CO_{3(aq)} (2 M, 0.5 mL) were flushed with N_{2(g)} for 5 min, then stirred under N_{2(g)} at 100° C. overnight. The reaction mixture was cooled to ambient temperature and evaporated to dryness. The residue was dissolved in H₂O, extracted with EtOAc (10 ml×3), the extracts were dried over MgSO₄ and filtered. The filtrate was passed through a silica gel pad, and the silica was washed with EtOAc. The solvent was removed to give a residue that was purified by radial chromatography (hexane/EtOAc v/v 95:5 to 80:20) to afford Compound 87 (92 mg, 80%) as an off-white solid: ¹H NMR (400 MHz, CDCl₃) δ 8.18 (d, 1H, J=8.4 Hz), 8.09 (s, 1H), 7.55 (d, 1H, J=7.6 Hz), 7.33 (m, 2H), 7.26 (m—obscured by CHCl₃ resonance, 1H), 6.57 (s, 1H), 3.86 (t, 4H, J=4.4 Hz), 3.54 (t, 4H, J=4.4 Hz), 1.45 (s, 9H); HRMS (ESI) [M+H]⁺ Calcd for C₂₂H₂₅O₃N₃F: 398.1875, found: 398.1863.

### 4-(4-Fluoro-5-(1H-indol-2-yl)pyridin-2-yl)morpholine (Compound 88)

Compound 85 (54 mg, 1.36×10⁻⁴ mol) was dissolved in TFA (2 mL), stirred at ambient temperature for 2 h, then the TFA was removed with N_{2(g)} flow. The residue was neutralized with aqueous NaHCO₃ solution to give a solid that was washed with cold CH₂Cl₂ to afford Compound 88 (30 mg, 74%) as an off-white solid: ¹H NMR (300 MHz, acetone-d₆) δ 10.52 (br s, 1H), 8.66 (d, 1H, J=11.4 Hz), 7.56 (d, 1H, J=8.1 Hz), 7.42 (d, 1H, J=8.1 Hz), 7.10 (partially resolved ddd, 1H, J=8.1 Hz, J=6.9 Hz, J=1.2 Hz), 7.02 (partially resolved ddd, 1H, J=7.8 Hz, J=7.2 Hz, J=1.2 Hz), 6.81 (m, 1H), 6.71 (d, 1H, J=14.7 Hz), 3.76 (apparent t, 4H, J=4.8 Hz), 3.59 (apparent t, 4H, J=4.8 Hz); ¹³C NMR (125 MHz, DMSO-d₆) δ 166.33 (d, J_{FC}=257.8 Hz), 159.93 (d, J_{FC}=11.4 Hz), 147.61 (d, J_{FC}=3.8 Hz), 136.49, 129.80, 128.42, 121.48, 119.78, 119.32, 111.12, 107.52 (d, J_{FC}=10.1 Hz), 100.69 (d, J_{FC}=6.3 Hz), 93.66 (d, J_{FC}=22.5 Hz), 65.81, 45.00; HRMS (ESI) [M+H]⁺ Calcd for C₁₇H₁₇ON₃F: 298.1350, found: 298.1343.

### 2-(5-Fluoro-6-(piperidin-1-yl)pyridin-3-yl)-1H-indole (Compound 89)

Compound 86 (0.11 g, 2.78×10⁻⁴ mol) was dissolved in TFA (2 mL), stirred at ambient temperature for 2 h, then the TFA was removed with N_{2(g)} flow. The residue was neutralized with aqueous NaHCO₃ to give a solid which was washed with cold CH₂Cl₂ to afford Compound 89 (78 mg, 95%) as an off-white solid: ¹H NMR (300 MHz, acetone-d₆) δ 10.61 (br s, 1H), 8.50 (t, 1H, J=1.8 Hz), 7.80 (dd, 1H, J=14.7 Hz, J=1.8 Hz), 7.54 (apparent d, 1H, J=8.1 Hz), 7.38 (dd, 1H, J=8.7 Hz, J=0.6 Hz), 7.10 (ddd, 1H, J=8.1 Hz, J=6.9 Hz, J=1.2 Hz), 7.02 (ddd, 1H, J=8.1 Hz, J=6.9 Hz, J=1.2 Hz), 6.85 (m, 1H), 3.50 (m, 4H), 1.67 (m, 6H); ¹³C NMR (125 MHz, DMSO-d₆): 149.03 (d, J_{FC}=255.2 Hz), 148.34 (d, J_{FC}=6.4 Hz), 139.27 (d, J_{FC}=4.6 Hz), 137.00, 134.25, 128.59, 121.49, 120.81 (d, J_{FC}=2.8 Hz), 119.82 (d, J_{FC}=20.8 Hz), 119.80, 119.43, 111.08, 98.44, 48.25 (d, J_{FC}=5.8 Hz), 25.37, 24.19; HRMS (ESI) [M+H]⁺ Calcd for C₁₈H₁₉N₃F: 296.1558, found: 296.1550.

### 4-(3-Fluoro-5-(1H-indol-2-yl)pyridin-2-yl)morpholine (Compound 90)

Compound 87 (90 mg, 2.26×10⁻⁴ mol) was dissolved in TFA (2 mL),stirred at ambient temperature for 1 h, then the TFA was removed with N_{2(g)} flow. The residue was neutralized with aqueous NaHCO₃ to give a solid that was purified by radial chromatography to afford Compound 90 (60 mg, 89%) as a solid: ¹H NMR (300 MHz, acetone-d₆) δ 10.65 (br s, 1H), 8.54 (apparent t, 1H, J=1.8 Hz), 7.86 (dd, 1H, J=14.4 Hz, J=1.8 Hz), 7.55 (d, 1H, J=7.8 Hz), 7.39 (partially resolved dd, 1H, J=8.1 Hz, J=0.9 Hz), 7.11 (ddd, 1H, J=8.7 Hz, J=6.9 Hz, J=1.2 Hz), 7.02 (partially resolved ddd, 1H, J=8.1 Hz, J=7.8 Hz, J=1.2 Hz), 6.88 (m, 1H), 3.78 (apparent t, 4H, J=4.8 Hz), 3.50 (apparent t, 4H, J=4.8 Hz); ¹³C NMR (125 MHz, DMSO-d₆): 149.29 (d, J_{FC}=255.4 Hz), 147.87 (d, J_{FC}=6.9 Hz), 139.24 (d, J_{FC}=4.8 Hz), 137.09, 136.93, 133.83 (d, J_{FC}=10.2 Hz), 128.49 (d, J_{FC}=10.4 Hz), 121.65, 120.08 (d, J_{FC}=20.5 Hz), 119.88, 119.49, 111.10, 98.78, 66.01, 47.73 (d, J_{FC}=5.4 Hz); HRMS (ESI) [M+H]⁺ Calcd for C₁₇H₁₇₀N₃F: 298.1350, found: 298.1348.

### tert-Butyl 2-(3-fluoro-5-(piperidin-1-yl)pyridin-2-yl)-1H-indole-1-carboxylate (Compound 91)

Compound 6 (0.22 g, 8.43×10⁻⁴ mol), compound 60 (90 mg, 4.19×10⁻⁴ mol), Pd(dppf)Cl₂ (19 mg, 2.60×10⁻⁵ mol), 1,4-dioxane (10 mL), and K₂CO_{3(aq)} (2 M, 0.5 mL) were flushed with N_{2(g)} for 5 min, then stirred under N₂(_{g} at 100° C. overnight (TLC indicated unreacted Compound 60 remaining). Additional Compound 6 (90 mg, 3.45×10⁻⁴ mol) was added and stirring under N_{2(g} at 100° C. continued to overnight, then the reaction mixture was cooled to ambient temperature and evaporated to dryness. The residue was dissolved in H₂O, extracted with EtOAc (10 ml×3), the extracts were dried over MgSO₄, and filtered. The filtrate was passed through a silica gel pad, and the silica was washed with EtOAc. The solvent was removed to give a residue that was purified by radial chromatography (hexane/EtOAc v/v 95:5 to 80:20) to afford Compound 91 (75 mg, 45%) as a solid: ¹H NMR (300 MHz, CDCl₃) δ 8.19 (dd, 1H, J=8.4 Hz, J=0.6 Hz), 8.15 (dd, 1H, J=2.7 Hz, J=1.5 Hz), 7.57 (d, 1H, J=7.5 Hz), 7.33 (ddd, 1H, J=8.4 Hz, J=7.2 Hz, J=1.2 Hz), 7.23 (apparent td—partially obscured by CHCl₃ resonance, 1H, J=7.5 Hz, J=0.9 Hz), 6.91 (dd, 1H, J=12.6 Hz, J=2.4 Hz), 6.78 (s, 1H), 3.28 (m, 4H), 1.70 (m, 6H), 1.42 (s, 9H); HRMS (ESI) [M+H]⁺ Calcd for C₂₃H₂₇O₂N₃F: 396.2082, found: 396.2082.

### 2-(3-Fluoro-5-(piperidin-1-yl)pyridin-2-yl)-1H-indole (Compound 92)

Compound 91 (75 mg, 1.90×10⁻⁴ mol) was dissolved in TFA(1 mL), stirred at ambient temperature for 1 h, then the TFA was removed with N_{2(g)} flow. The residue was neutralized with aqueous NaHCO₃ to give a solid which was washed with cold CH₂Cl₂ to afford Compound 92 (44 mg, 78%) as an off-white solid: ¹H NMR (300 MHz, acetone-d₆) δ 10.60 (br s, 1H), 8.20 (t, 1H, J=2.4 Hz), 7.58 (partially resolved dd, 1H, J=8.1 Hz), 7.55 (partially resolved dd, 1H, J=8.1 Hz, J=0.9 Hz), 7.19 (dd, 1H, J=14.7 Hz, J=2.4 Hz), 7.12 (ddd, 1H, J=8.1 Hz, J=7.2 Hz, J=1.2 Hz), 7.01 (ddd, 1H, J=7.8 Hz, J=7.2 Hz, J=1.2 Hz), 6.94 (m, 1H), 3.37 (m, 4H), 1.70 (m, 6H); ¹³C NMR (125 MHz, DMSO-d₆): 157.07 (d, J_{FC}=258.7 Hz), 147.40 (d, J_{FC}=5.4 Hz), 136.20, 132.75 (d, J_{FC}=7.2 Hz), 132.42, 128.70, 127.30 (d, J_{FC}=12.2 Hz), 121.70, 120.22, 119.15, 111.69, 108.77 (d, J_{FC}=22.6 Hz), 100.67 (d, J_{FC}=11.9 Hz), 48.18, 24.72, 23.64; HRMS (ESI) [M+H]⁺ Calcd for C₁₈H₁₉N₃F: 296.1558, found: 296.1562.

### tert-Butyl 2-(3-fluoro-5-morpholinopyridin-2-yl)-1H-indole-1-carboxylate (Compound 93)

Compound 6 (0.17 g, 6.51×10⁻⁴ mol), compound 61 (78 mg, 3.60×10⁻⁴ mol), Pd(dppf)Cl₂ (18 mg, 2.46×10⁻⁵ mol), 1,4-dioxane (10 mL), and K₂CO_{3(aq)} (2 M, 0.5 mL) were flushed with N_{2(g)} for 5 min, then stirred under N_{2(g)} at 100° C. overnight. The reaction mixture was cooled to ambient temperature and evaporated to dryness. The residue was dissolved in H₂O, extracted with EtOAc (10 ml×3), the extracts were dried over MgSO₄, and filtered. The filtrate was passed through a silica gel pad, and the silica was washed with EtOAc. The solvent was removed to give a residue that was purified by radial chromatography (hexane/EtOAc v/v 95:5 to 80:2) to afford recovered Compound 61 (58 mg, 74%) and Compound 93 (29 mg, 20%) as a solid: ¹H NMR (400 MHz, CDCl₃) δ 8.18 (d, 1H, J=8.4 Hz), 8.15 (s, 1H), 7.58 (d, 1H, J=7.6 Hz), 7.34 (apparent t, 1H, J=7.6 Hz), 7.24 (apparent t, 1H, J=7.2 Hz), 6.92 (dd, 1H, J=12.0 Hz, J=2.0 Hz), 6.81 (s, 1H), 3.90 (t, 4H, J=4.8 Hz), 3.26 (t, 4H, J=4.8 Hz), 1.43 (s, 9H); HRMS (ESI) [M+H]⁺ Calcd for C₂₂H₂₅O₃N₃F: 398.1875, found: 398.1865.

### tert-Butyl 6-fluoro-2-(3-fluoro-5-morpholinopyridin-2-yl)-1H-indole-1-carboxylate (Compound 94)

Compound 76 (0.19 g, 6.81×10⁻⁴ mol), compound 61 (87 mg, 4.02×10⁻⁴ mol), Pd(dppf)Cl₂ (19 mg, 2.60×10⁻⁵ mol,), 1,4-dioxane (10 mL), and K₂CO_{3(aq)} (2 M, 0.5 mL) were flushed with N_{2(g)} for 5 min, then stirred under N_{2(g)} at 100° C. overnight (TLC indicated unreacted Compound 61 remaining). Additional Compound 76 (93 mg, 3.33×10⁻⁴ mol) was added, the reaction mixture was stirred under N_{2(g)} at 100° C. for 5 h, then cooled to ambient temperature and evaporated to dryness. The residue was dissolved in H₂O, extracted with EtOAc (10 ml×3), the extracts were combined, dried over MgSO₄, and filtered. The filtrate was passed through a silica gel pad, and the silica was washed with EtOAc. The solvent was removed to give a residue that was purified by radial chromatography (hexane/EtOAc v/v 95:5 to 80:20) to afford recovered Compound 61 (23 mg, 26%) and Compound 94 (96 mg, 57%) as a solid: ¹H NMR (400 MHz, CDCl₃) δ 8.15 (s, 1H), 7.91 (partially resolved dd, 1H, J=10.8 Hz, J=1.2 Hz), 7.49 (dd, 1H, J=8.4 Hz, J=5.6 Hz), 7.00 (apparent td, 1H, J=9.6 Hz, J=1.6 Hz), 6.92 (apparent dd, 1H, J=12.4 Hz, J=1.6 Hz), 6.76 (s, 1H), 3.90 (t, 4H, J=4.8 Hz), 3.26 (t, 4H, J=4.8 Hz), 1.43 (s, 9H); HRMS (ESI) [M+H]Calcd for C₂₂H₂₄O₃N₃F₂: 416.1780, found: 416.1767.

### tert-Butyl 2-(4-fluoro-5-morpholinopyridin-2-yl)-1H-indole-1-carboxylate (Compound 95)

Compound 6 (85 mg, 3.26×10⁻⁴ mol), compound 62 (78 mg, 3.60×10⁻⁴ mol), Pd(dppf)Cl₂ (18 mg, 2.46×10⁻⁵ mol), 1,4-dioxane (5 mL), and K₂CO_{3(aq)} (2 M, 0.5 mL) were stirred at 100° C. under N_{2(g)} overnight. The reaction mixture was cooled to ambient temperature, evaporated to dryness, the residue was dissolved in H₂O and extracted with EtOAc (10 ml×3). The combined extracts were dried over MgSO₄, filtered, and the filtrate was passed through a silica gel pad and washed with EtOAc. The solution was concentrated to dryness and the residue was purified by radial chromatography (hexane/EtOAc v/v 95:5 to 70:30) to give recovered Compound 62 (32 mg, 41%) and compound 95 (45 mg, 35%): ¹H NMR (300 MHz, CDCl₃) δ 8.28 (d, 1H, J=11.1 Hz), 8.15 (d, 1H, J=8.1 Hz), 7.57 (d, 1H, J=7.8 Hz), 7.35 (partially resolved ddd, 1H, J=8.4 Hz, J=7.2 Hz, J=1.2 Hz), 7.25 (m—partially obscured by CHCl₃ resonance, 1H), 7.19 (d, 1H, J=12.9 Hz), 6.75 (s, 1H), 3.90 (apparent t, 4H, J=4.5 Hz), 3.21 (apparent t, 4H, J=4.5 Hz), 1.42 (s, 9H); HRMS (ESI) [M+H]⁺ Calcd for C₂₂H₂₅O₃N₃F: 398.1875, found: 398.1865.

### tert-Butyl 6-fluoro-2-(4-fluoro-5-morpholinopyridin-2-yl)-1H-indole-1-carboxylate (Compound 96)

Compound 76 (0.23 g, 8.24×10⁴ mol), compound 62 (97 mg, 4.48×10⁻⁴ mol), Pd(dppf)Cl₂ (18 mg, 2.46×10⁻⁵ mol), 1,4-dioxane (10 mL), and K₂CO_{3(aq)} (2 M, 0.5 mL) were flushed with N_{2(g)} for 5 min, then stirred under N_{2(g)} and heated at 100° C. overnight. The reaction mixture was cooled to ambient temperature, evaporated to dryness, the residue was dissolved in H₂O and extracted with EtOAc (10 ml×3). The combined extracts were dried over MgSO₄, filtered, and the filtrate was passed through a silica gel pad and washed with EtOAc. The solution was concentrated to dryness and the residue was purified by radial chromatography (hexane/EtOAc v/v 95:5 to 80:20) to afford recovered Compound 62 (68 mg, 70%) and Compound 96 (29 mg, 16%) as a solid: ¹H NMR (300 MHz, CDCl₃) δ 8.27 (d, 1H, J=10.8 Hz), 7.88 (dd, 1H, J=10.5 Hz, J=2.4 Hz), 7.48 (dd, 1H, J=8.7 Hz, J=5.4 Hz), 7.18 (d, 1H, J=12.9 Hz), 7.01 (td, 1H, J=9.0 Hz, J=2.4 Hz), 6.70 (s, 1H), 3.90 (m—AA′XX′, 4H, J=4.8 Hz, J=1.8 Hz), 3.21 (m—AA′XX′, 4H, J=4.8 Hz, J=1.8 Hz), 1.41 (s, 9H); HRMS (ESI) [M+H]⁺ Calcd for C₂₂H₂₄O₃N₃F₂: 416.1780, found: 416.1767.

### 4-(5-Fluoro-6-(1H-indol-2-yl)pyridin-3-yl)morpholine (Compound 97)

Compound 93 (29 mg, 7.30×10⁻⁵ mol) was dissolved in TFA (1 mL), stirred at ambient temperature for 2 h, then the TFA was removed with N_{2(g)} flow. The residue was neutralized with aqueous NaHCO₃ to give a solid that was washed with cold CH₂Cl₂ to afford Compound 97 (17 mg, 78%) as an off-white solid: ¹H NMR (400 MHz, acetone-d₆) δ 10.65 (br s, 1H), 8.22 (t, 1H, J=2.0 Hz), 7.60 (unresolved dq, 1H, J=8.0 Hz), 7.56 (partially resolved dq, 1H, J=8.0 Hz, J=0.8 Hz), 7.24 (dd, 1H, J=14.4 Hz, J=2.4 Hz), 7.14 (ddd, 1H, J=8.8 Hz, J=7.2 Hz, J=1.2 Hz), 7.02 (ddd, 1H, J=7.8 Hz, J=7.2 Hz, J=1.2 Hz), 6.98 (dm, 1H, J=4.4 Hz), 3.81 (apparent t, 4H, J=4.8 Hz), 3.32 (partially resolved m—AA′XX′, 4H, J=4.8 Hz); ¹³C NMR (125 MHz, DMSO-d₆): 156.94 (d, J_{FC}=258.6 Hz), 147.20 (d, J_{FC}=4.3 Hz), 136.24, 132.53 (d, J_{FC}=6.4 Hz), 132.14, 128.65, 128.46 (d, J_{FC}=10.9 Hz), 121.88, 120.33, 119.22, 111.74, 108.95 (d, J_{FC}=22.1 Hz), 101.06 (d, J_{FC}=11.9 Hz), 65.69, 47.23; HRMS (ESI) [M+H]⁺ Calcd for C₁₇H₁₇₀N₃F: 298.1350, found: 298.1353.

### 4-(5-Fluoro-6-(6-fluoro-1H-indol-2-yl)pyridin-3-yl)morpholine (Compound 98)

Compound 94 (31 mg, 7.46×10⁻⁵ mol) was dissolved in TFA(1 mL), stirred at ambient temperature for I h, then the TFA was removed with N_{2(g)} flow. The residue was neutralized with aqueous NaHCO₃ to give a solid that was washed with cold CH₂Cl₂ to afford Compound 98 (23 mg, 98%) as an off-white solid: ¹H NMR (300 MHz, acetone-d₆) δ 10.77 (br s, 1H), 8.21 (t, 1H, J=1.8 Hz), 7.58 (dd, 1H, J=8.7 Hz, J=5.4 Hz), 7.30 (dd, 1H, J=10.2 Hz, J=2.4 Hz), 7.23 (dd, 1H, J=14.4 Hz, J=2.4 Hz), 6.96 (dm, 1H, J=4.2 Hz), 6.85 (ddd, 1H, J=9.9 Hz, J=8.7 Hz, J=2.4 Hz), 3.81 (apparent t, 4H, J=4.8 Hz), 3.32 (apparent t, 4H, J=4.8 Hz), ¹³C NMR (125 MHz, DMSO-d₆): 159.18 (d, J_{FC}=235.3 Hz), 156.82 (d, J_{FC}=258.8 Hz), 147.26 (d, J_{FC}=5.5 Hz), 136.24 (d, J_{FC}=13.2 Hz), 133.33 (unresolved m), 132.13 (d, J_{FC}=2.8 Hz), 128.15 (d, J_{FC}=11.9 Hz), 125.49, 121.46 (d, J_{FC}=10.2 Hz), 108.93 (d, J_{FC}=22.8 Hz), 107.83 (d, J_{FC}=24.5 Hz), 101.06 (d, J_{FC}=12.1 Hz), 97.51 (d, J_{FC}=25.7 Hz), 65.69, 47.21; HRMS (ESI) [M+H]⁺ Calcd for C₁₇H₁₆ON₃F₂: 316.1256, found: 316.1258.

### 4-(4-Fluoro-6-(1H-indol-2-yl)pyridin-3-yl)morpholine (Compound 99)

Compound 95 (40 mg, 1.01×10⁻⁴ mol) was dissolved in TFA (1.5 mL), stirred at ambient temperature for 1.5 h, then the TFA was removed with N_{2(g)} flow. The residue was neutralized with aqueous NaHCO₃ to give a solid that was washed with hexanes and cold CH₂Cl₂ to afford Compound 99 (29 mg, 97%) as an off-white solid: ¹H NMR (300 MHz, acetone-d₆) δ 10.73 (br s, 1H), 8.28 (d, 1H, J=11.1 Hz), 7.72 (d, 1H, J=13.8 Hz), 7.58 (d, 1H, J=8.1 Hz), 7.53 (d, 1H, J=8.1 Hz, J=0.9 Hz), 7.14 (ddd, 1H, J=8.1 Hz, J=7.2 Hz, J=1.2 Hz), 7.07 (m, 1H), 7.02 (ddd, 1H, J=7.8 Hz, J=7.2 Hz, J=0.9 Hz), 3.82 (m—AA′XX′, 4H, J=4.8 Hz, J=1.8 Hz), 3.19 (m—AA′XX′, 4H, J=4.8 Hz, J=1.8 Hz); ¹³C NMR (125 MHz, DMSO-d₆): 160.55 (d, J_{FC}=258.7 Hz), 146.18 (d, J_{FC}=8.0 Hz), 140.75 (unresolved d), 137.10, 136.37 (unresolved d), 134.62 (d, J_{FC}=5.2 Hz), 128.33, 122.15, 120.43, 119.43, 111.87, 107.55 (d, J_{FC}=18.4 Hz), 100.24, 65.99, 50.08; HRMS (ESI) [M+H]⁺ Calcd for C₁₇H₁₇ON₃F: 298.1350, found: 298.1347.

### 4-(4-Fluoro-6-(6-fluoro-1H-indol-2-yl)pyridin-3-yl)morpholine (Compound 100)

Compound 96 (29 mg, 6.98×10⁻⁵ mol) was dissolved in TFA (1 mL), stirred at ambient temperature for 2 h, then the TFA was removed with N_{2(g)} flow. The residue was neutralized with aqueous NaHCO₃ to give a solid which was washed with cold CH₂Cl₂ to afford Compound 100 (21 mg, 95%) as an off-white solid: ¹H NMR (300 MHz, acetone-d₆) δ 10.86 (br s, 1H), 8.27 (d, 1H, J=11.1 Hz), 7.71 (d, 1H, J=13.8 Hz), 7.57 (dd, 1H, J=8.7 Hz, J=5.4 Hz), 7.26 (dd, 1H, J=9.9 Hz, J=2.4 Hz), 7.08 (m, 1H), 6.85 (ddd, 1H, J=9.9 Hz, J=8.7 Hz, J=2.4 Hz), 3.82 (m—AA′XX′, 4H, J=4.8 Hz, J=1.8 Hz), 3.19 (m—AA′XX′, 4H, J=4.8 Hz, J=1.8 Hz), ¹³C NMR (125 MHz, DMSO-d₆): 160.56 (d, J_{FC}=258.8 Hz), 159.30 (d, J_{FC}=235.7 Hz), 145.85 (d, J_{FC}=8.3 Hz), 140.71 (d, J_{FC}=3.0 Hz), 137.16 (dd, J_{FC}=3.6 Hz), 137.03 (d, J_{FC}=13.2 Hz), 134.69 (d, J_{FC}=5.8 Hz), 125.17, 121.59 (d, J_{FC}=10.2 Hz), 108.11 (d, J_{FC}=24.5 Hz), 107.46 (d, J_{FC}=18.5 Hz), 100.26, 97.63 (d, J_{FC}=25.8 Hz), 65.99, 50.07 (d, J_{FC}=2.6 Hz); HRMS (ESI) [M+H]⁺ Calcd for C₁₇H₁₆ON₃F₂: 316.1256, found: 316.1245.

### tert-Butyl 2-(2-(4-(2-(2-fluoroethoxy)ethoxy)piperidin-1-yl)pyrimidin-5-yl)-1H-indole-1-carboxylate (Compound 101)

Compound 44 (79 mg, 2.00×10⁻⁴ mol) was flushed with N_{2(g)}, dissolved in DMA (1 mL), and cooled to 0° C. NaH (60%, 26 mg, 6.50×10⁻⁴ mol, 3.2 equiv.) was added, the mixture was stirred at 0° C. under N_{2(g)} for 5 min, then a solution of 2-(2-fluoroethoxy)ethyl 4-methylbenzenesulfonate (110 mg, 4.19×10⁻⁴ mol, 2.1 equiv.) in DMA (0.5 mL) was added. The reaction mixture was stirred at ambient temperature under N_{2(g)} for 2 h, then H₂O (5 drops) was added, the mixture was stirred for 10 min, then poured into a mixture of H₂O (20 mL), EtOAc (20 mL), hexane (10 mL). The layers were mixed, separated, and the organic layer was washed with H₂O (10 mL×2), brine (10 mL), and dried over MgSO₄. The solvent was removed to give a yellow residue (138 mg) that was dissolved in CH₂Cl₂, poured onto dry silica (55 cm h×45 cm i.d.), and eluted under vacuum: CH₂Cl₂ (100 mL), % MeOH/CH₂Cl₂—1% (100 mL), 2% (100 mL), 3% (100 mL), 5% (100 mL) to give a faint brown syrup (126 mg). Purification by radial chromatography (1 mm silica): CH₂Cl₂ (50 mL), % MeOH/CH₂Cl₂—1% (50 mL), 2% (25 mL) afforded Compound 101 (32 mg, 33%) as a colorless residue: ¹H NMR (300 MHz, CDCl₃) δ 8.48 (s, 2H), 7.62 (d, 1H, J=7.8 Hz), 7.41 (d, 1H, J=8.1 Hz), 7.24 (td, 1H, J=8.1 Hz, J=0.9 Hz), 7.15 (partially resolved td, 1H, J=7.8 Hz, J=0.9 Hz), 6.50 (s, 1H), 4.86 (apparent septet, 1H, J=4.2 Hz), 4.52 (apparent t, 1H, J=4.2 Hz), 4.39 (m, 3H), 4.30 (t, 2H, J=6.0 Hz), 3.80 (t, 2H, J=6.0 Hz), 3.55 (m, 4H), 2.06 (m, 2H), 1.77 (m, 2H), 1.52 (s, 9H).

### 2-(2-(4-(2-(2-Fluoroethoxy)ethoxy)piperidin-1-yl)pyrimidin-5-yl)-1H-indole (Compound 102)

Compound 101 (31 mg, 6.40×10⁻⁵ mol) and TFA (0.65 mL, 8.4 mmol, 132 equiv.) were stirred for 20 min, then poured into a mixture of NaHCO₃ (0.79 g, 9.40 mmol, 1.1 equiv. TFA), H₂O (25 mL), and CH₂Cl₂ (25 mL). The mixture was stirred for 5 min, the layers were separated, and the aqueous layer was extracted with CH₂Cl₂ (5 mL×2). The combined CH₂Cl₂ layers were washed with brine (20 mL), dried over MgSO₄, concentrated, poured onto dry silica (33 mm h×33 mm i.d.), and eluted under vacuum: % MeOH/CH₂Cl₂—1% (50 mL), 2% (75 mL), 3% (100 mL) to afford Compound 102 (16 mg, 65%) as an off-white solid: ¹H NMR (MHz, CDCl₃) δ 8.48 (s, 2H), 7.62 (d, 1H, J=7.6 Hz), 7.41 (d, 1H, J=8.0 Hz), 7.24 (td, 1H, J=8.0 Hz, J=0.8 Hz), 7.15 (partially resolved td, 1H, J=7.6 Hz, J=0.8 Hz), 6.50 (s, 1H), 4.48 (dt, 2H, J=13.4 Hz, J=4.4 Hz), 4.44 (dt, 2H, ²J_{HF}=48.0 Hz, J=4.4 Hz), 4.30 (t, 2H, J=6.0 Hz), 4.01 (apparent septet, 1H, J=4.4 Hz), 3.80 (t, 2H, J=6.0 Hz), 3.55 (dt, 2H, ³J_{HF}=29.2 Hz, J=4.4 Hz), 3.42 (ddd, 2H, J=13.4 Hz, J=10.0 Hz, J=3.6 Hz), 2.01 (m, 2H), 1.58 (m, 2H); HRMS (ESI) [M+H]⁺ Calcd for C₂₁H₂₆FN₄O₂: 385.2034, found: 385.2041.

### 4-(5-(6-Fluoro-1-methyl-1H-indol-2-yl)pyrimidin-2-yl)morpholine (Compound 103)

Compound 2 (21 mg, 7.04×10⁻⁵ mol) was flushed with N₂(_{g}>then dissolved in DMA (1 mL) and cooled to 0° C. NaH (60%, 9.6 mg, 2.40×10⁻⁴ mol, 3.4 equiv.) was added, the reaction mixture was stirred at 0° C. under N_{2(g)} for 18 min, then MeOTs (26.5 mg, 1.42×10⁻⁴ mol, 2 equiv.) was added. The reaction mixture was warmed to ambient temperature, stirred for 2 h, then H₂O (1 drop) was added. The reaction mixture was combined with EtOAc (15 mL), hexane (5 mL), and H₂O (10 mL), the layers were mixed and separated, and the organic layer was washed with H₂O (10 mL×2), brine (10 mL), and dried over MgSO₄. The solvent was removed to give an off-white solid that was dissolved in CH₂Cl₂, poured onto dry silica (33 mm h×33 mm i.d.), and eluted under vacuum: % MeOH/CH₂Cl₂—1% (100 mL), 2% (50 mL), 3% (50 mL) to afford Compound 103 (19 mg, 86%) as an off-white solid: ¹H NMR (400 MHz, CDCl₃) δ 8.46 (s, 2H), 7.52 (dd, 1H, J=8.4 Hz, J=5.4 Hz), 7.02 (dd, 1H, J=10.0 Hz, J=2.0 Hz), 6.91 (td, 1H, J=9.0 Hz, J=2.0 Hz), 6.49 (s, 1H), 3.88 (m, 4H), 3.81 (m, 4H), 3.67 (s, 3H); HRMS (ESI) [M+H]⁺ Calcd for C₁₇H₁₈FN₄O: 313.1459, found: 313.1455.

### tert-Butyl 6-fluoro-1H-indole-1-carboxylate (Compound 104)

6-Fluoroindole (3.28 g, 24.27 mmol), Boc₂O (6.59 g, 30.19 mmol, 1.2 equiv.), i-Pr₂NEt (7 mL, 40.2 mmol, 1.7 equiv.), DMAP (0.31 g, 2.54 mmol, 0.1 equiv.) and CH₂Cl₂ (210 mL) were stirred at ambient temperature under N_{2(g)} for 16 h. The reaction mixture was concentrated to a yellow/orange oil, hexane was added, and the mixture was purified by vacuum flash chromatography on silica (16 cm h×4 cm i.d.): hexane (100 mL), % CH₂Cl₂/hexane—10% (200 mL), 25% (400 mL) to afford Compound 104 (5.67 g, 99%) as a colorless oil: ¹H NMR (300 MHz, CDCl₃) δ 7.86 (br d, 1H, J=10.2 Hz), 7.56 (d, 1H, J=3.6 Hz), 7.46 (dd, 1H, J=8.4 Hz, J=5.4 Hz), 6.98 (td, 1H, J=9.0 Hz, J=2.4 Hz), 6.53 (d, 1H, J=3.6 Hz), 1.67 (s, 9H).

### Example 3. In Vitro Binding Assays

All screened compounds were >95% purity as determined by analytical HPLC. Binding assays were performed as previously described (Graham, T. J. A., et al, Journal of Medicinal Chemistry 2023, 66, 10628-10638), and results are shown in Table 2.

TABLE 2
Inhibition Constant (Kᵢ) Values of Ligands versus [³H]1 and
[³H]2 in AD, PSP, and CBD Brain Tissue Homogenates.^{a}

| | [³H]1 | | | [³H]2 | | |
|---|---|---|---|---|---|---|
| | | Kᵢ (nM) | | | Kᵢ (nM) | |
| | AD | PSP | CBD | AD | PSP | CBD |
| Compound | tissue | tissue | tissue | tissue | tissue | tissue |
| 15 | 3.5 | 6.2 | 6.3 | 7.7 | 9.2 | 12 |
| 16 | 11 | 14 | 11 | 14 | 18 | 15 |
| 17 | 37 | 23 | n.d. | n.d. | n.d. | n.d. |
| 18 | 120 | 129 | n.d. | 74 | 76 | n.d. |
| 21 | 9.4 | 8.9 | 9.0 | 11 | 13 | 10 |
| 22 | 39 | 32 | n.d. | 34 | 31 | n.d. |
| 36 | 5.0 | 10 | 8.2 | 9.5 | 10 | 14 |
| (R)-37 | 16 | 14 | 15 | 17 | 23 | 20 |
| 38 | 12 | 13 | 17 | 17 | 14 | 15 |
| 39 | 4.6 | 4.6 | 6.2 | 4.9 | 5.2 | 5.9 |
| 40 | 9.5 | 7.9 | 11 | 13 | 11 | 12 |
| 43 | 45 | 91 | n.d. | n.d. | n.d. | n.d. |
| 46 | 11 | 24 | n.d. | n.d. | n.d. | n.d. |
| 47 | 21 | 20 | n.d. | n.d. | n.d. | n.d. |
| 68 | 8.0 | 6.6 | 6.4 | 7.8 | 7.7 | 7.8 |
| 69 | 9.2 | 10 | 13 | 6.5 | 10 | 11 |
| 72 | n.d. | n.d. | n.d. | 9.2 | 9.5 | 10 |
| 73 | n.d. | n.d. | n.d. | 5.0 | 6.3 | 7.7 |
| 75 | n.d. | n.d. | n.d. | 29 | 34 | 26 |
| 81 | n.d. | n.d. | n.d. | 8.0 | 7.3 | 8.2 |
| 82 | n.d. | n.d. | n.d. | 11 | 12 | 11 |
| 83 | n.d. | n.d. | n.d. | 7.5 | 7.8 | 8.7 |
| 84 | n.d. | n.d. | n.d. | 24 | 20 | 18 |
| 88 | n.d. | n.d. | n.d. | 13 | 15 | 12 |
| 89 | n.d. | n.d. | n.d. | 16 | 17 | 14 |
| 90 | n.d. | n.d. | n.d. | 7.9 | 8.6 | 9.5 |
| 92 | n.d. | n.d. | n.d. | 15 | 20 | 16 |
| 97 | n.d. | n.d. | n.d. | 21 | 25 | 22 |
| 98 | n.d. | n.d. | n.d. | 17 | 20 | 20 |
| 99 | n.d. | n.d. | n.d. | 50 | 44 | 49 |
| 100 | n.d. | n.d. | n.d. | 115 | 65 | 56 |
| 102 | 19 | 20 | 39 | 20 | 23 | 30 |
| 103 | 22 | 32 | n.d. | n.d. | n.d. | n.d. |

^{a}n = 1. n.d. = not determined.

### Example 4. In Vitro Competition (Kᵢ) Assays

The equilibrium inhibition constant (Kᵢ) values of the compounds were determined versus tritium-labeled radioligands using published methods (Klunk et al., “The binding of 2-(4′-methylaminophenyl)benzothiazole to postmortem brain homogenates is dominated by the amyloid component.” J Neurosci 2003, 23, 2086-2092). The assays utilized autopsy-confirmed postmortem AD, PSP, and CBD human brain tissues obtained from the UCSF Neurodegenerative Disease Brain Bank and contained frequent autopsy-confirmed 3R/4R-tau and amyloid-beta aggregates (AD tissue) or only 4R-tau aggregates (PSP and CBD tissues) and no other detectable aggregated amyloid species. Briefly, the tissues were homogenized in ice-cold pH 7.0 phosphate buffered saline (PBS) at 300 mg/mL on ice using a glass homogenizer, diluted 30-fold with PBS to 10 mg/mL and homogenized a second time with a Brinkmann Polytron homogenizer before storage at −80° C. Frozen brain tissue was thawed and diluted 10-fold in PBS to 1 mg/mL. The concentration of unlabeled competitor compound (-400 M in the stock solution) was determined by quantitative NMR in DMSO (0.25% DMSO in the final assay vials). The appropriate concentrations (ranging from 0.1-1000 nM) of unlabeled competitor in 400 μL of PBS buffer were combined with 500 μL of the tritium-labeled radioligand in PBS (~1 nM final concentration of radioligand). The assay was initiated by the addition of 100 μL of 1 mg/mL brain tissue homogenate to achieve a final concentration of 100 pg tissue/mL. After incubation for 60 min at room temperature, the binding mixture was filtered through a Whatman GF/B glass filter via a Brandel M-24R cell harvester (Gaithersburg, MD, USA) and rapidly washed four times with 3 mL PBS buffer. The filters were counted in Cytoscint-ES after thorough vortexing using a liquid scintillation counter. Complete (100%) inhibition of specific binding was defined as the number of counts displaced by 1 μM unlabeled competitor. All assays were performed in triplicate at each concentration. The Kᵢ value was determined by the concentration of inhibitor that resulted in 50% binding inhibition (IC₅₀) of the radioligand: Kᵢ=IC₅₀/2.

### Example 5. Binding Affinity (K_{d}) Assays

Tritium-labeled radioligand binding assays utilized brain AD, PSP, or CBD homogenates to determine equilibrium dissociation constant (K_{d}) values and were performed with slight modifications of the procedure previously described in detail (Klunk et al., “Uncharged thioflavin-T derivatives bind to amyloid-beta protein with high affinity and readily enter the brain.” Life Sciences 2001, 69 (13), 1471-1484). Briefly, frozen aliquots (−80° C.) of homogenized cortex (10 mg/mL in PBS (pH=7.0)) from AD, PSP, or CBD brain were thawed and diluted 10-fold in PBS to 1 mg/mL. The unlabeled test compound was dissolved in DMSO at 400 M and then diluted to 20 M with PBS to yield 5% DMSO/PBS. The remaining serial dilutions (typically from 6 μM to 4 nM) were made with 5% DMSO/PBS to maintain a constant DMSO concentration in the final assay. Fifty μL of these solutions were combined with 50 μL of tritiated test compound and 800 μL of PBS to yield 0.25% DMSO, −1 nM tritiated compound and 0.2 to 1000 nM unlabeled compound in the final assay. The assay began by addition of 100 μL of the 1 mg/mL brain homogenate to achieve a final concentration of 100 μg tissue/mL. After incubation for 60 min at room temperature, the binding mixture was filtered through a Whatman GF/B glass filter via a Brandel M-24R cell harvester (Gaithersburg, MD) and rapidly washed three times with 3 mL PBS. The filters were counted in Cytoscint-ES after thorough vortexing and sitting overnight. All assays were performed at least in triplicate. The concentration of bound compound was determined from the radioactivity retained on the filter after correcting for the non-displaceable radioactivity (defined as that remaining with ~1 μM unlabeled compound) and the specific activity of the tritiated compound after dilution with varying concentrations of unlabeled compound. The K_{d} value was determined by the slope (slope=−1/K_{d}) of a Scatchard plot of the bound/free vs. bound radioligand values at the different ligand concentrations.

### Example 6. X-Ray Crystal Structure Data

TABLE 3
Sample and crystal data for compound 38 (JSS20-116A).

| Identification code | JSS20-116A | |
|---|---|---|
| Chemical formula | C₁₇H₁₅FN₄O | |
| Formula weight | 310.33 g/mol | |
| Temperature | 296(2) K | |
| Wavelength | 1.54178 Å | |
| Crystal size | 0.003 × 0.120 × 0.200 mm | |
| Crystal habit | clear colorless plate | |
| Crystal system | monoclinic | |
| Space group | P 1 21/n 1 | |
| Unit cell dimensions | a = 12.5190(4) Å | α = 90° |
| | b = 7.8750(4) Å | β = 90.611(3)° |
| | c = 15.0667(5) Å | γ = 90° |

| | |
|---|---|
| Volume | 1485.30(10) Å³ |
| Z | 4 |
| Density (calculated) | 1.388 g/cm³ |
| Absorption coefficient | 0.817 mm⁻¹ |
| F(000) | 648 |

TABLE 4
Sample and crystal data for compound 65 (JSS21-021B).

| Identification code | JSS21-021B | |
|---|---|---|
| Chemical formula | C₂₃H₂₆N₄O₄ | |
| Formula weight | 422.48 g/mol | |
| Temperature | 296(2) K | |
| Wavelength | 1.54178 Å | |
| Crystal size | 0.050 × 0.120 × 0.190 mm | |
| Crystal habit | clear yellow Chunk | |
| Crystal system | orthorhombic | |
| Space group | P 21 21 21 | |
| Unit cell dimensions | a = 10.3939(2) Å | α = 90° |
| | b = 12.8366(3) Å | β = 90° |
| | c = 16.0313(4) Å | γ = 90° |

| | |
|---|---|
| Volume | 2138.93(8) Å³ |
| Z | 4 |
| Density (calculated) | 1.312 g/cm³ |
| Absorption coefficient | 0.747 mm⁻¹ |
| F(000) | 896 |

TABLE 5
Sample and crystal data for compound 66 (JSS21-033A).

| Identification code | JSS21-033A | |
|---|---|---|
| Chemical formula | C₂₂H₂₄N₄O₅ | |
| Formula weight | 424.45 g/mol | |
| Temperature | 150(2) K | |
| Wavelength | 1.54178 Å | |
| Crystal size | 0.004 × 0.120 × 0.200 mm | |
| Crystal system | monoclinic | |
| Space group | P 1 21/c 1 | |
| Unit cell dimensions | a = 8.68420(10) Å | α = 90° |
| | b = 12.4480(2) Å | β = 91.5310(10)° |
| | c = 19.3489(3) Å | γ = 90° |

| | |
|---|---|
| Volume | 2090.89(5) Å³ |
| Z | 4 |
| Density (calculated) | 1.348 g/cm³ |
| Absorption coefficient | 0.805 mm⁻¹ |
| F(000) | 896 |

TABLE 6
Sample and crystal data for compound 69 (JSS20-183A).

| Identification code | JSS20-183A | |
|---|---|---|
| Chemical formula | C₁₇H₁₆FN₃O | |
| Formula weight | 297.33 g/mol | |
| Temperature | 100(2) K | |
| Wavelength | 0.71073 Å | |
| Crystal size | 0.050 × 0.120 × 0.170 mm | |
| Crystal habit | clear colorless block | |
| Crystal system | monoclinic | |
| Space group | P 1 21/c 1 | |
| Unit cell dimensions | a = 16.3426(5) Å | α = 90° |
| | b = 5.9494(2) Å | β = 105.0520(10)° |
| | c = 14.4881(4) Å | γ = 90° |

| | |
|---|---|
| Volume | 1360.33(7) Å³ |
| Z | 4 |
| Density (calculated) | 1.452 g/cm³ |
| Absorption coefficient | 0.102 mm⁻¹ |
| F(000) | 624 |

### Example 7. Radiosynthesis and In Vitro Evaluation of [³H]1 ([³H]Z-3540) and [³H]2 ([³H]Z-2340) in Tauopathy Tissue Samples

Z-3540 (Enamine Z3777013540, Compound 1) and Z-2340 (Enamine Z4169252340, Compound 2) were identified through a molecular similarity fingerprint search of the Enamine compound library based on CBD-2115. [³H]Z-3540 and [³H]Z-2340 were synthesized and evaluated in binding assays in human CBD, PSP and AD tissues.

In human tissues, the K_{d} of [³H]Z-3540 was 3-fold lower in AD and 7-fold lower in CBD than [3H]CBD-2115. [3H]Z-3540 was found to be selective for binding aggregated tau over b-amyloid and a-synuclein.

### Example 8. Radiochemistry

The X-ray crystal structures of radiolabeling precursors 65 and 66 are shown in FIG. 2 and FIG. 3, respectively. Radiolabeling of [¹⁸F]68 and [¹⁸F]69 was performed manually in a lead-shielded hot cell using manipulator arms. [¹⁸F]Fluoride was trapped on a Waters Sep-Pak Plus Light QMA, then eluted with Et₄NHCO_{3(aq)} to give Et₄N¹⁸F which was then azeotropically dried with CH₃CN. Nitro-precursors 65 or 66 were dissolved in DMSO, added to the dried Et₄N¹⁸F in a sealed 5-mL V-vial, heated at ~140° C. for 30 minutes, then the crude reaction mixture was purified by semi-preparatory HPLC. Radiotracer [¹⁸F]68 was obtained in 4.4% non-decay-corrected radiochemical yield (1.18 GBq (31.9 mCi)); 99.4% radiochemical purity; molar activity of Aₘ=0.13 GBq/nmol (3.5 mCi/nmol); in a 10% EtOH/0.9% sterile saline solution, pH 5. Radiotracer [¹⁸F]69 was obtained in 11.5% non-decay-corrected radiochemical yield (3.49 GBq (94.3 mCi)); 98.9% radiochemical purity; molar activity of Aₘ=0.05 GBq/nmol (1.4 mCi/nmol); in a 10% EtOH/0.9% sterile saline solution, pH 5; log D=1.96±10.03 (n=4). Radiochemical identity of the radiotracers was confirmed by analytical HPLC co-injection with the unlabeled standards.

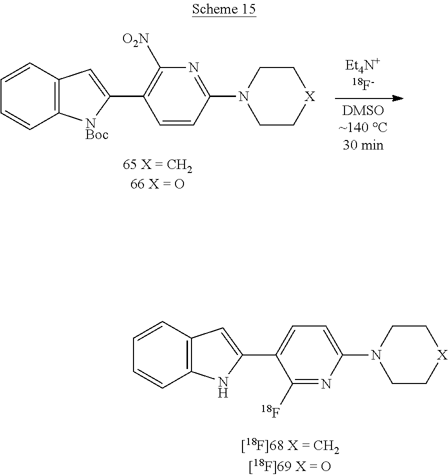

### 2-(2-[¹⁸F]Fluoro-6-(piperidin-1-yl)pyridin-3-yl)-1H-indole ([¹⁸F]68)

Et₄NHCO₃ (70 mg) was dissolved in Milli-Q H₂O (10 mL) to give a 7 mg/mL solution. A Waters Sep-Pak Plus Light QMA (WAT023525) was rinsed with Et₄NHCO_{3(aq)} solution (8 mL, 7 mg/mL), then Milli-Q H₂O (10 mL), then air (25 mL). A Waters C₁₈ Sep-Pak (WAT020515) was rinsed with EtOH (10 mL) then Milli-Q H₂O (10 mL). No-carrier added [¹⁸F]F⁻ was produced via the ¹⁸O(p,n)¹⁸F reaction (Siemens Eclipse HP cyclotron) and transferred in [¹⁸O]H₂O to a lead-shielded hot cell. [¹⁸F]F⁻ (26.6 GBq (719 mCi)) was trapped on the QMA and eluted with Et₄NHCO_{3(aq)} solution (1 mL, 7 mg/mL), then CH₃CN (1 mL) (0.45 GBq (12.2 mCi) remained on the QMA), and collected in a 5-mL V-vial. The solvents were evaporated under argon flow at 110° C., then the Et₄N¹⁸F was azeotropically dried with CH₃CN aliquots (1 mL×3) at 110° C. under argon flow. Compound 65 (3.8 mg) was dissolved in DMSO (0.6 mL), added to the dried Et₄N¹⁸F, the V-vial was capped and mixed gently for ~10 seconds until the solution turned faint pink, then heated at ~140° C. for 30 min. The V-vial was cooled for −1 min, the solution was diluted with HPLC solvent (0.5 mL, 55:45 v/v CH₃CN/0.1 M NH₄HCO₂ pH 4.2), loaded onto the HPLC loop, the V-vial was rinsed with HPLC solvent (0.5 mL), the solvent was loaded onto the HPLC loop, and the sample was purified by prep-HPLC (Phenomenex Gemini, 250×10 mm+guard; 5 mL/min for 6 min, then 10 mL/min). The HPLC radioactive peak at ~24-25 min was collected, diluted with Milli-Q H₂O (50 mL), passed through the Cis Sep-Pak, and the Sep-Pak was rinsed with Milli-Q H₂O (10 mL). The radiotracer was eluted from the Sep-Pak with EtOH (1 mL), then 0.9% sterile saline (9 mL), passed through a 0.2 micrometer filter, and collected in a sterile vial to afford [¹⁸F]68 (1.18 GBq (31.9 mCi); 4.4% radiochemical yield (not decay-corrected); 99.4% radiochemical purity (Phenomenex Gemini, 5 micrometer, NX-C18 110 Å, 100×4.6 mm, 60:40 v/v CH₃CN/0.1 M NH₄HCO₂ pH 4.2; 1 mL/min; 254 nm); Aₘ=0.13 GBq/nmol (3.5 mCi/nmol); pH 5). Radiochemical identity was confirmed by co-injection with 68. The endotoxin level of the final formulation was <2.00 EU/mL (Charles Rivers Endosafe®).

4-(6-[¹⁸F]Fluoro-5-(1H-indol-2-yl)pyridin-2-yl)morpholine ([⁸F]69) ([⁸F]JSS20-183A). Et₄NHCO₃ (70 mg) was dissolved in Milli-Q H₂O (10 mL) to give a 7 mg/mL solution. A Waters Sep-Pak Plus Light QMA (WAT023525) was rinsed with Et₄NHCO_{3(aq)} solution (8 mL, 7 mg/mL), then Milli-Q H₂O (10 mL), then air (25 mL). A Waters Cis Sep-Pak (WAT020515) was rinsed with EtOH (10 mL) then Milli-Q H₂O (10 mL). No-carrier added [¹⁸F]F⁻ was produced via the ¹⁸O(p,n)¹⁸F reaction (Siemens Eclipse HP cyclotron) and transferred in [¹⁸O]H₂O to a lead-shielded hot cell. [¹⁸F]F⁻ (30.3 GBq (818 mCi)) was trapped on the QMA and eluted with Et₄NHCO_{3(aq)} solution (1 mL, 7 mg/mL), then CH₃CN (1 mL), then air (1 mL) (0.3 GBq (8.0 mCi) remained on the QMA), and collected in a 5-mL V-vial. The solvents were evaporated under argon flow at 110° C., then the Et₄N¹⁸F was azeotropically dried with CH₃CN aliquots (1 mL×3) at 110° C. under argon flow. Compound 66 (2.5 mg) was dissolved in DMSO (0.5 mL), added to the dried Et₄N¹⁸F (the solution turned light purple), the V-vial was capped, and heated at −140° C. for 30 min. The V-vial was cooled for ~1 min, the solution was diluted with HPLC solvent (0.5 mL, 45:55 v/v CH₃CN/0.1 M NH₄HCO₂ pH 4.2), loaded onto the HPLC loop, the V-vial was rinsed with HPLC solvent (0.5 mL), the solvent was loaded onto the HPLC loop, and the sample was purified by prep-HPLC (Phenomenex Gemini, 250×10 mm+guard; 5 mL/min for 6 min, then 8 mL/min). The HPLC radioactive peak at ~20.4-22 min was collected, diluted with Milli-Q H₂O (50 mL), passed through the C₁₈ Sep-Pak, and the Sep-Pak was rinsed with Milli-Q H₂O (10 mL). The radiotracer was eluted from the Sep-Pak with EtOH (1 mL), then 0.9% sterile saline (9 mL), passed through a 0.2 micrometer filter, and collected in a sterile vial to afford [¹⁸F]69 (3.49 GBq (94.3 mCi); 11.5% radiochemical yield (not decay-corrected); 98.9% radiochemical purity (Phenomenex Gemini, 5 micrometer, NX-C18 110 Å, 100×4.6 mm; 45:55 v/v CH₃CN/0.1 M NH₄HCO₂ pH 4.2; 1 mL/min; 254 nm); Aₘ=0.05 GBq/nmol (1.4 mCi/nmol); pH 5; log D=1.96±0.03 (n=4)). Radiochemical identity was confirmed by co-injection with 69. The endotoxin level of the final formulation was <2.00 EU/mL (Charles Rivers Endosafe @).

### Example 9. Binding Affinity of [³H]69 ([³H]JSS20-183A)

The binding affinity of [³H]69 ([³H]JSS20-183A) was determined in post-mortem brain tissue (Table 7). [³H]69 ([³H]JSS20-183A) shows high affinity in post-mortem human AD, PSP, CBD, and Pick's Disease brain tissue, and also in P301L mouse brain tissue. [³H]69 ([3H]JSS20-183A) shows low affinity in Parkinson's Disease (PD) post-mortem brain tissue, thus demonstrating that 69 does not bind strongly to alpha-synuclein. Compound 69 does not compete well against [³H]PiB in post-mortem human AD brain tissue, thus demonstrating that 69 does bind to the PiB binding site on amyloid-beta.

TABLE 7
Homologous binding assay determination
of [³H]69 ([³H]JSS20-183A ) binding affinities
in post-mortem brain tissues, and competition
of compound 69 against [³H]PiB in
post-mortem human AD brain tissue.

| K_{D} (nM) | | | | | | Kᵢ (nM) |
|---|---|---|---|---|---|---|
| | | | | P301L | | vs. |
| | | | | | | [³H]PiB |
| AD | PSP | CBD | Pick's | Mouse | PD | in AD |
| Tissue | Tissue | Tissue | Tissue | Tissue | Tissue | Tissue |
| 1.5 ± 0.4 | 2.3 ± 0.6 | 2.5 ± 0.3 | 3.3 ± 0.3 | 2.8 ± 0.7 | 110 ± 54 | 61 ± 3 |

(n = 4)

### Example 10. Brain PET Imaging Studies

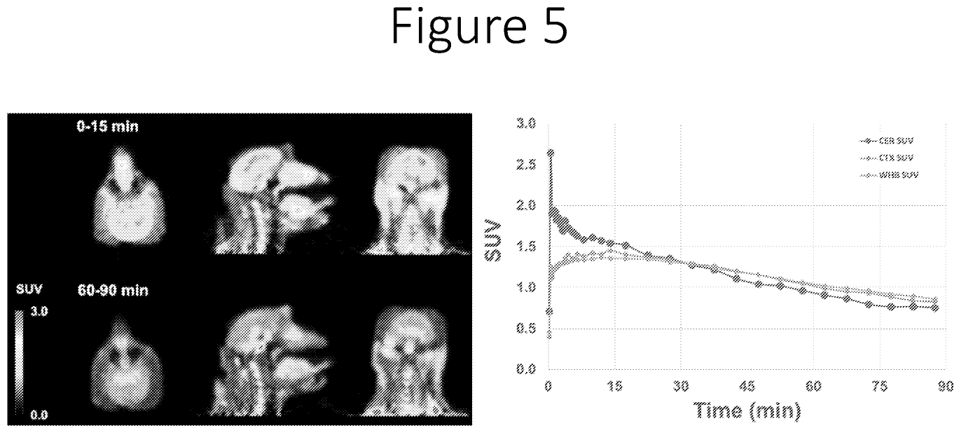

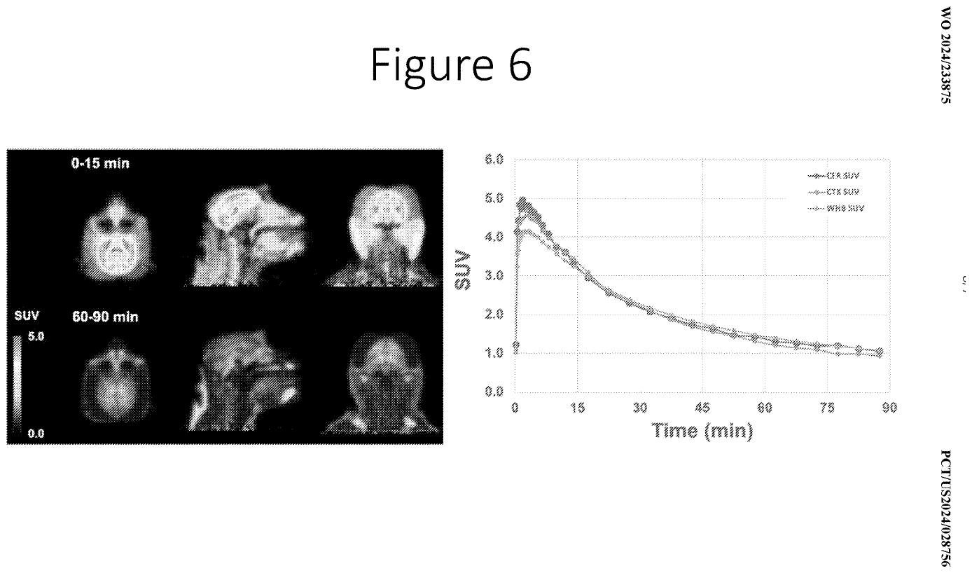

Brain uptake and washout of [⁸F]68 (FIG. 5) and [⁸F]69 (FIG. 6) was evaluated by brain PET imaging studies in rhesus macaques. The 2-minute to 60-minute ratios of [¹⁸F]68 were 1.2 (whole brain), 1.3 (cortex), and 2.0 (cerebellum). The 2-minute to 60-minute ratios of [¹⁸F]69 were 3.0 (whole brain), 3.7 (cortex), and 3.8 (cerebellum).

### Example 11. Autoradiography

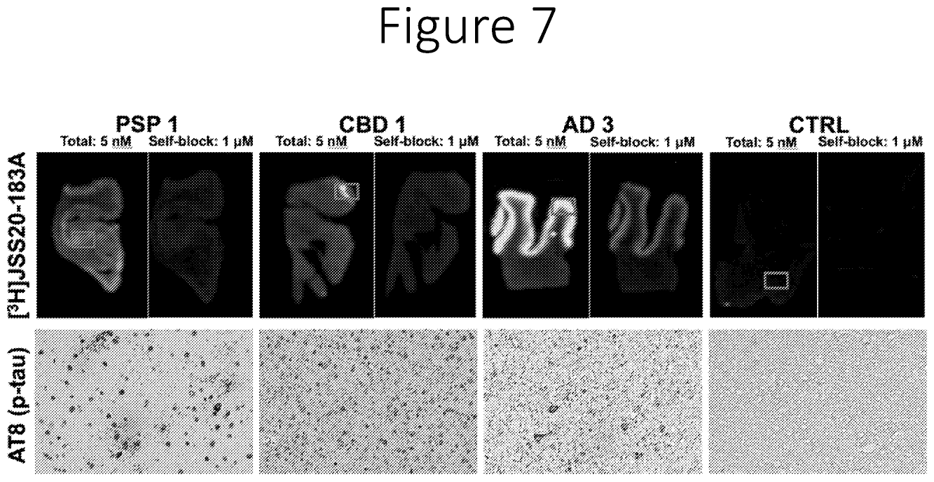

Specific binding of [³H]69 in post-mortem human PSP, CBD, and AD brain tissue was demonstrated by autoradiography (FIG. 7). Little to no specific binding was observed in post-mortem human brain control tissue.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A compound having a structure of Formula I or Formula II, or a pharmaceutically acceptable salt, solvate, hydrate, tautomer or stereoisomer thereof: 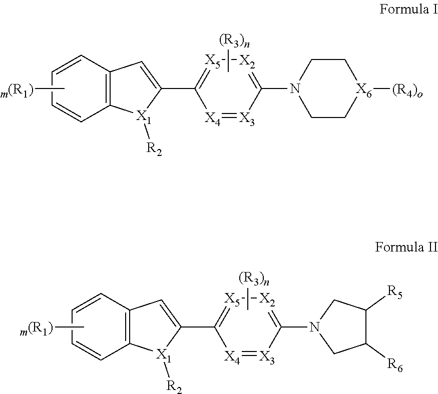
wherein:
X₁ is N, O or S;
X₂, X₃, X₄, X₅, are each independently N or CH;
X₆ is CH or O;
R₁ is H, halogen, CN, Me₃N⁺X₇⁻, Et₃N⁺X₇⁻, Pr₃N⁺X₇⁻, Bu₃N⁺X₇⁻, or other suitable ammonium salt, OH, OMs, OTs, OTf, O-nosylate, O-brosylate, or other suitable sulfonate species, SnMe₃, SnEt₃, SnPr₃, SnBu₃, or other suitable trialkyltin species, I(O-acyl)₂, ArI⁺X₇⁻, IR₇, S(O)Ar, SO₂Ar, BF₃K, B(OH)₂, B(OMe)₂, B(OEt)₂, B(Opr)₂, B(OiPr)₂, B(OiPr)₃Li, B-pinacol, B-neopentyl glycol, or other suitable boron species, with the proviso that when R₁ is OH, R₁ is not substituted at indole ring position 5;
wherein X₇⁻ is a suitable counterion; wherein ArI⁺X₇⁻ is a suitable iodonium species known in the art; wherein IR₇ is a suitable iodonium ylide known in the art;
R₂ is H, alkyl, tert-butoxycarbonyl, tosyl, benzyl, p-methoxybenzyl, CBz, Fmoc, or other suitable nitrogen protecting group;
R₃ is H, halogen, NO₂, Me₃N⁺X₇⁻, Et₃N⁺X₇⁻, Pr₃N⁺X₇⁻, Bu₃N⁺X₇⁻, or other suitable ammonium salt, OH, OMs, OTs, OTf, O-nosylate, O-brosylate, or other suitable sulfonate species, SnMe₃, SnEt₃, SnPr₃, SnBu₃, or other suitable trialkyltin species, I(O-acyl)₂, ArI⁺X₇⁻, IR₇, S(O)Ar, SO₂Ar, BF₃K, B(OH)₂, B(OMe)₂, B(OEt)₂, B(Opr)₂, B(OiPr)₂, B(OiPr)₃Li, B-pinacol, B-neopentyl glycol, or other suitable boron species;
wherein X₇⁻ is a suitable counterion; wherein ArI⁺X₇⁻ is a suitable iodonium species known in the art; wherein IR₇ is a suitable iodonium ylide known in the art;
R₄ is H, halogen, OH, alkoxy, carbonyl, or OEtOEtF;
R₅ is H, halogen, OH, OMs, OTs, OTf, O-nosylate, O-brosylate, or other suitable sulfonate species;
R₆ is H, halogen, OH, O-acyl, O-alkyl, OBoc, or other suitable oxygen protecting species;
or R₅ and R₆ form an epoxide: 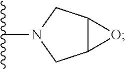 [SMILES: *N1CC2OC2C1]
m and n are each independently 0 or 1; and
o is 0, 1, or 2.

2. The compound of claim 1 having the following structure:

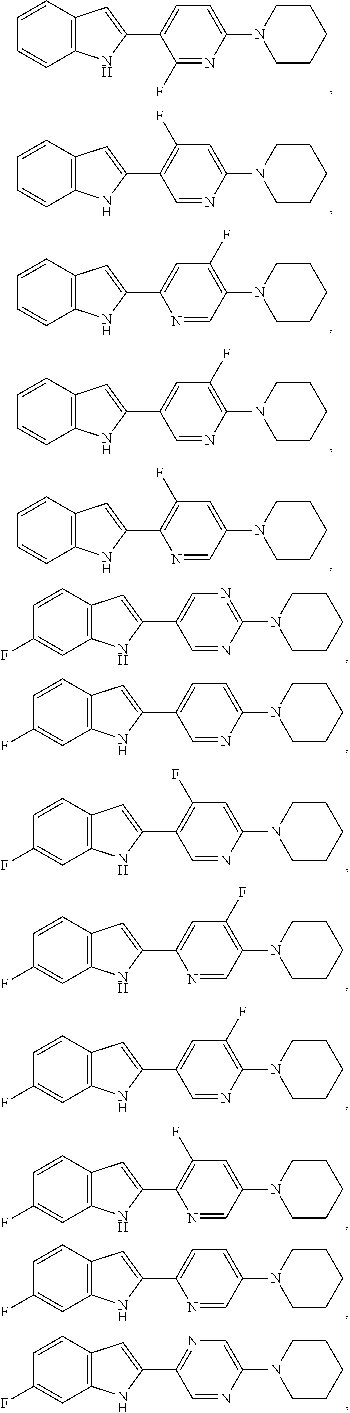

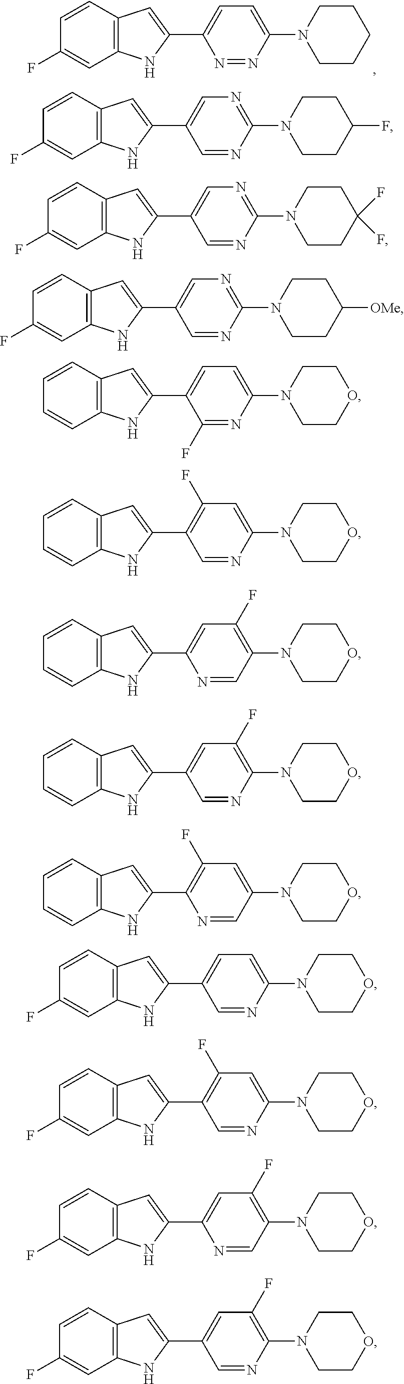

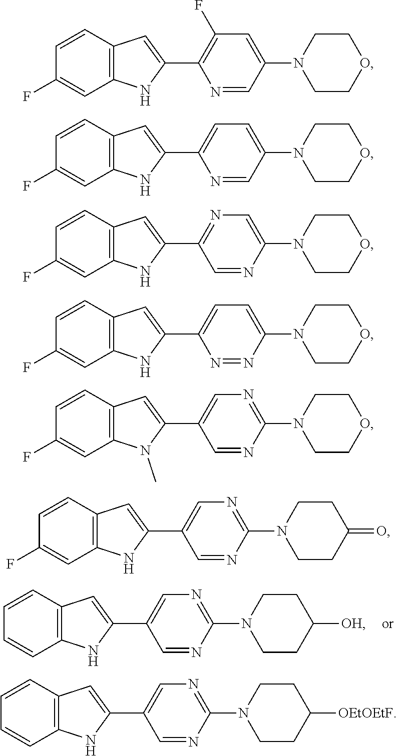

3. The compound of claim 1 having the following structure:

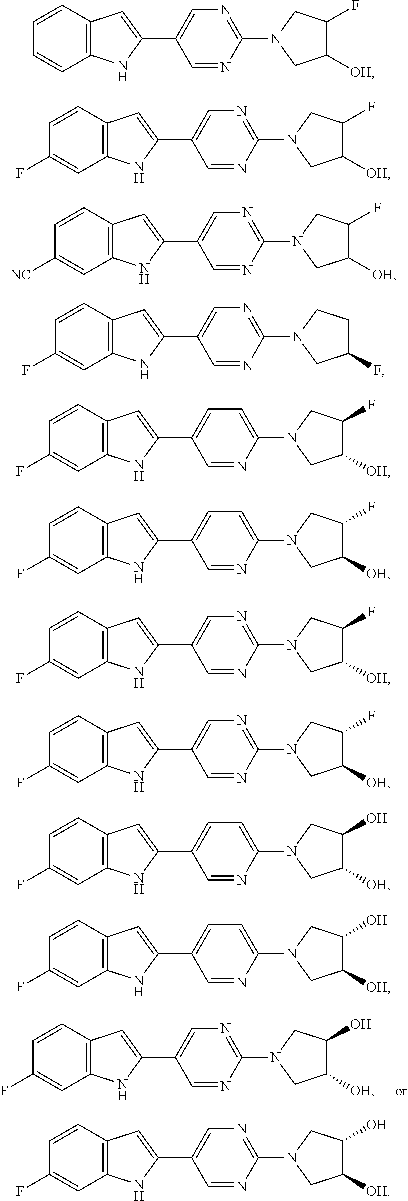

4. The compound of claim 1, wherein the compound selectively binds to 4R tau.

5. The compound of claim 1, wherein the compound comprises one or more detection labels.

6. The compound of claim 5, wherein the detection label is selected from the group consisting of a radionuclide, a positron emitter, a gamma emitter, and a fluorescent label.

7. The compound of claim 5, wherein the detection label is selected from the group consisting of ²H, ³H, ¹⁸F, ¹²³I, ¹²⁴I, ¹²⁵I, ¹³¹I, ¹¹C, ¹³C, ¹⁴C, ¹³N, ¹⁵N, ¹⁵O, ⁷⁵Br, ⁷⁶Br, and ⁷⁷Br.

8. The compound of claim 5, wherein the detection label is ¹⁸F.

9.-13. (canceled)

14. The method of claim 15, wherein detection is carried out by positron emission tomography (PET) imaging.

15. A method for diagnosing a subject with a disease or disorder, or monitoring disease or disorder progression in a subject, the method comprising:
(a) administering to the subject a detectable quantity of an isotopically labeled compound of Formula I or Formula II, or a pharmaceutically acceptable salt, solvate, hydrate, tautomer or stereoisomer thereof: 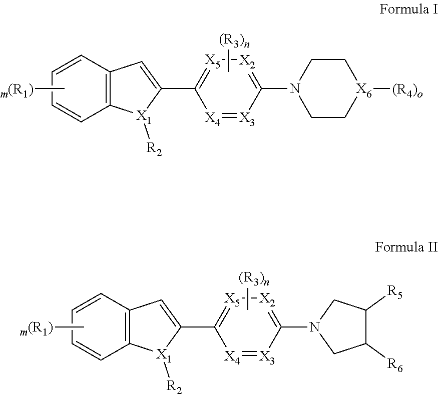
wherein:
X₁ is N, O or S;
X₂, X₃, X₄, X₅, are each independently N or CH;
X₆ is CH or O;
R₁ is H, halogen, CN, Me₃N⁺X₇⁻, Et₃N⁺X₇⁻, Pr₃N⁺X₇⁻, Bu₃N⁺X₇⁻, or other suitable ammonium salt, OH, OMs, OTs, OTf, O-nosylate, O-brosylate, or other suitable sulfonate species, SnMe₃, SnEt₃, SnPr₃, SnBu₃, or other suitable trialkyltin species, I(O-acyl)₂, ArI⁺X₇⁻, IR₇, S(O)Ar, SO₂Ar, BF₃K, B(OH)₂, B(OMe)₂, B(OEt)₂, B(Opr)₂, B(OiPr)₂, B(OiPr)₃Li, B-pinacol, B-neopentyl glycol, or other suitable boron species, with the proviso that when R₁ is OH, R₁ is not substituted at indole ring position 5;
wherein X₇⁻ is a suitable counterion; wherein ArI⁺X₇⁻ is a suitable iodonium species known in the art; wherein IR₇ is a suitable iodonium ylide known in the art;
R₂ is H, alkyl, tert-butoxycarbonyl, tosyl, or other suitable nitrogen protecting group;
R₃ is H, halogen, NO₂, Me₃N⁺X₇⁻, Et₃N⁺X₇⁻, Pr₃N⁺X₇⁻, Bu₃N⁺X₇⁻, or other suitable ammonium salt, OH, OMs, OTs, OTf, O-nosylate, O-brosylate, or other suitable sulfonate species, SnMe₃, SnEt₃, SnPr₃, SnBu₃, or other suitable trialkyltin species, I(O-acyl)₂, ArI⁺X₇⁻, IR₇, S(O)Ar, SO₂Ar, BF₃K, B(OH)₂, B(OMe)₂, B(OEt)₂, B(Opr)₂, B(OiPr)₂, B(OiPr)₃Li, B-pinacol, B-neopentyl glycol, or other suitable boron species;
wherein X₇⁻ is a suitable counterion; wherein ArI⁺X₇⁻ is a suitable iodonium species known in the art; wherein IR₇ is a suitable iodonium ylide known in the art;
R₄ is H, halogen, OH, alkoxy, carbonyl, or OEtOEtF;
R₅ is H, halogen, OH, OMs, OTs, OTf, O-nosylate, O-brosylate, or other suitable sulfonate species;
R₆ is H, halogen, OH, O-acyl, O-alkyl, OBoc, or other suitable oxygen protecting species;
or R₅ and R₆ form an epoxide: 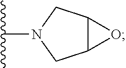 [SMILES: *N1CC2OC2C1]
m and n are each independently 0 or 1; and
o is 0, 1, or 2,
and
(b) detecting binding of the compound to tau aggregates in the patient.

16. The method of claim 15, wherein the disease or disorder is associated with tau aggregates.

17. The method of claim 16, wherein the disease or disorder is a 4R-tauopathy or a disease or disorder correlated with aggregates of 4R tau.

18. The method of claim 16, wherein the disease or disorder is a neurodegenerative disease or disorder.

19. The method of claim 15, wherein the disease or disorder is selected from the group consisting of Alzheimer's disease, progressive supranuclear palsy, Pick's disease, frontal temporal lobar dementias (FTLD's), frontotemporal dementia with parkinsonism-17 (FTDP-17), Primary age-related tauopathy (PART) dementia, argyrophilic grain disease, glial globular tauopathy, chronic traumatic encephalopathy (CTE), corticobasal syndrome (CBS), and corticobasal degeneration.

20. The method of claim 15, wherein the compound is isotopically labeled with ²H, ³H, ¹⁸F, ¹²³I, ¹²⁴I, ¹²⁵I, ¹³¹I, ¹¹C, ¹³C, ¹⁴C, ¹³N, ¹⁵N, ¹⁵O, ⁷⁵Br, ⁷⁶Br, and ⁷⁷Br.

21. The method of claim 15, wherein detection is carried out carried out by one or more of positron emission tomography (PET) imaging, single photon emission computed tomography (SPECT), magnetic resonance imaging, or autoradiography.
